(19) United States
(12) Patent Application Publication
(10) Pub. No.: US 2010/0256214 A1
(43) Pub. Date: Oct. 7, 2010

(54) EPH RECEPTOR LIGANDS AND METHODS OF USE

(75) Inventors: Elena B. Pasquale, San Diego, CA (US); Roberta Noberini, San Diego, CA (US); Mitchell Koolpe, Santa Fe, NM (US); Jianxing Song, Singapore (SG)

Correspondence Address: PATENT CORRESPONDENCE;ARNALL GOLDEN GREGORY LLP, 171 17TH STREET NW, SUITE 2100, ATLANTA, GA, 30363, US

(73) Assignee: Burnham Institute for Medical Research, La Jolla, CA (US)

(21) Appl. No.: 12/751,203

(22) Filed: Mar. 31, 2010

Related U.S. Application Data
(60) Provisional application No. 61/166,046, filed on Apr. 2, 2009.

(51) Int. Cl. A61K 31/40; G01N 33/53; C12Q 1/02; C40B 30/04; A61K 31/34

(52) U.S. Cl. 514/408; 435/7.1; 435/29; 506/9; 514/461

(57) ABSTRACT

Disclosed are methods and compositions relating to binder, modulators and inhibitors of EphA4 and EphA2.

### CROSS-REFERENCE TO RELATED APPLICATIONS

This application claims benefit to U.S. Provisional Application No. 61/166,046, filed on Apr. 2, 2009, and is hereby incorporated herein in its entirety.

### STATEMENT REGARDING FEDERALLY SPONSORED RESEARCH

This invention was made with government support under National Institutes of Health grants R03NS 053627, X01 MH077609, P01 HD025938, and R01 CA116099. The government has certain rights in the invention.

### REFERENCE TO SEQUENCE LISTING

The Sequence Listing submitted herewith as a text file named “24520_{—}15_{—}8402_{—}2010_{—}03_{—}31_AMD_AFD_Sequence_Listing_ST25.txt,” created on Mar. 29, 2010, and having a size of 23 kilobytes is hereby incorporated by reference pursuant to 37 C.F.R. §1.52(e)(5).

### FIELD OF THE INVENTION

The disclosed invention is generally in the field of Eph receptors and specifically in the area of inhibition of Eph receptors.

### BACKGROUND OF THE INVENTION

Receptor protein tyrosine kinases (PTKs) are a structurally related family of proteins that mediate the response of cells to extracellular signals (Ullrich et al. Cell 61, 203 212 (1990)). These receptors are characterized by three major functional domains: an intracellular region containing the sequences responsible for catalytic activity, a single hydrophobic membrane-spanning domain, and a glycosylated extracellular region whose structure determines ligand binding specificity. Receptor protein kinases have been grouped into well-defined families on the basis of both sequence homology and shared structural motifs. The amino acid sequence of the portion of the intracellular domain responsible for the catalytic activity is well conserved among all tyrosine kinases and even more closely matched within a receptor sub-family. Comparisons of this portion of the amino acid sequence have been used to construct phylogenetic trees depicting the relatedness of family members to each other and to the tyrosine kinases as a whole (Hanks and Quinn, Methods Enzymol. 200, 38 62 (1991)).

There are 51 known distinct protein kinase receptor genes that have been published and divided into 14 sub-families, one such sub-family is the erythropoietin-producing hepato-cellular carcinoma (Eph)-like receptors. The prototype member, Eph, was isolated by Hirai et al. (Science 238, 1717 1720 (1987)) using low stringency hybridization to a probe derived from the viral oncogene v-fps. The Eph receptors constitute the largest family of receptor tyrosine kinases, with 16 individual receptors throughout the animal kingdom, which are activated by 9 ephrins (Adams, (2002) Semin Cell Dev Biol 13(1), 55-60; Pasquale, (2005) Nat Rev Mol Cell Biol 6(6), 462-475; Egea and Klein, (2007) Trends Cell Biol 17(5), 230-38; Luo and Flanagan, (2007) Neuron 56(2), 284-300; Heroult et al. (2006) Exp Cell Res 312(5), 642-650; Pasquale, (2008) Cell 133(1), 38-52). The Eph receptors have a modular structure, consisting of a unique N-terminal ephrin binding domain followed by a cysteine-rich linker and two fibronectin type III repeats in the extracellular region. The intracellular region is composed of a conserved tyrosine kinase domain, a C-terminal sterile α-domain, and a PDZ binding motif. The N-terminal 180-residue globular domain of the Eph receptors has been shown to be sufficient for high-affinity ephrin binding (Himanen et al. (1998) Nature 396, 486-491; Himanen et al. (2007) Curr Opin Cell Biol 19(5), 534-542; Himanen et al. (2001) Nature 414, 933-938). The amino acid sequences of the catalytic domains are more closely related to the SRC sub-family of cytoplasmic PTKs than to any of the receptor PTKs. Among the catalytic domains of receptor PTKs, the Eph sub-family is most similar in amino acid sequence to the epidermal growth factor receptor sub-family.

Eph receptors and their ligands are both anchored onto the plasma membrane, and are subdivided into two subclasses (A and B) based on their sequence conservation and binding preferences. EphA receptors (EphA1-A10) interact with glycosylphosphatidylinositol (GPI)-anchored ephrin-A ligands (ephrin-A1-A6), while EphB receptors (EphB1-B6) interact with transmembrane ephrin-B ligands (ephrin-B1-ephrin-B3) that have a short cytoplasmic portion carrying both SH2 and PDZ domain-binding motifs (Cowan and Henkemeyer, (2001) Nature 413, 174-179; Song, (2003) J. Biol. Chem. 278, 24714-24720). EphA subclass receptors remarkably differ from EphB receptors because they lack a 4-residue insert in the H-I loop of the ligand-binding domain. Previously, the structures of the EphB2 and EphB4 ligand-binding domains have been determined in both the free state and in complex with ephrins or peptide antagonists (Himanen et al. (2007) Curr Opin Cell Biol 19(5), 534-542; Himanen et al. (2001) Nature 414, 933-938; Himanen et al. (2004) Nat. Neurosci. 7, 501-9; Chrencik et al. (2006) Structure. 14, 321-30; Chrencik et al. (2006) J Biol. Chem. 281, 28185-92). The ligand-binding domains of EphB2 and EphB4 adopt the same jellyroll β-sandwich architecture composed of 11 antiparallel β-strands connected by loops of various lengths. In particular, the D-E and J-K loops have been revealed to play a critical role by forming the high-affinity Eph-ephrin binding channel. Interactions between Eph receptors and ephrins initiate bidirectional signals that direct pattern formation and morphogenetic processes, such as axon growth, cell assembly and migration, and angiogenesis (Adams, (2002) Semin Cell Dev Biol 13(1), 55-60; Pasquale, (2005) Nat Rev Mol Cell Biol 6(6), 462-475; Egea and Klein, (2007) Trends Cell Biol 17(5), 230-38; Luo and Flanagan, (2007) Neuron 56(2), 284-300; Heroult et al. (2006) Exp Cell Res 312(5), 642-650; Pasquale, (2008) Cell 133(1), 38-52; Cowan and Henkemeyer, (2001) Nature 413, 174-179; Song, (2003) J. Biol. Chem. 278, 24714-24720).

The Eph receptor tyrosine kinases regulate a variety of physiological and pathological processes not only during development but also in adult organs, and therefore represent a promising class of drug targets. Despite the numerous possible research and therapeutic applications of agents capable of modulating Eph receptor function, no small molecule inhibitors targeting the extracellular domain of these receptors have been identified. The Eph receptors have been extensively studied for their roles in the developing and injured nervous system and in the developing cardiovascular system (Adams, (2002) Semin Cell Dev Biol 13(1), 55-60, Pasquale, (2005) Nat Rev Mol Cell Biol 6(6), 462-475, Egea and Klein, (2007) Trends Cell Biol 17(5), 230-238, Luo and Flanagan, (2007) Neuron 56(2), 284-300, Du et al. (2007) Current pharmaceutical design 13(24), 2507-2518, Heroult et al. (2006) Exp Cell Res 312(5), 642-650). More recently, Eph receptors have also been implicated in many other physiological and pathological processes, including the regulation of insulin secretion, bone homeostasis, immune function, blood clotting, pathological forms of angiogenesis and cancer (Pasquale, (2008) Cell 133(1), 38-52). The ability to modulate the activities of this family of receptors is therefore of critical interest in order to gain a better understanding of their functions in the physiology of many organs and in various pathological conditions, as well as for medical therapy.

EphA4 is the most promiscuous member of the Eph family and can bind both ephiin-A and ephrin-B ligands (Ephrins A1, A2, A3, A4, A5, B2, and B3; Pasquale, Curr. Opin. in Cell Biology, 1997, 9: 608; Pasquale, Nat. Neurosci. 7:417, 2004). Ligand binding leads to EphA4 autophosphorylation on tyrosine residues (Ellis, et al., Oncogene 12: 1727, 1996), creating a binding region for proteins with Src Homology 2/3 (SH2/SH3) domains, such as the cytoplasmic tyrosine kinase p59fyn (Ellis et al., supra; Cheng, et al., Cytokine and Growth Factor Reviews 13: 75, 2002), EphA4 is expressed in brain, heart, lung, muscle, kidney, placenta, pancreas (Fox et al, Oncogene 10: 897, 1995) and melanocytes (Easty et al., Int. J. Cancer 71: 1061, 1997). The EphA4 receptor was shown to have important roles in the inhibition of the regeneration of injured axons, synaptic plasticity, platelet aggregation and likely in certain types of cancer. Activation of EphA4 in Xenopus embryos leads to loss of cadherin-dependent cell adhesion (Winning et al. Differentiation 70: 46, 2002 Cheng et al. (2002)), which is a property of metastatic cancer, supporting a possible role for EphA4 in tumor progression. EphA4 is upregulated in breast cancer, esophageal cancer, and pancreatic cancer (Kuan et al., Nucleic Acids Res, 26: 1116, 1998; Meric et al, Clinical Cancer Res. 8: 361, 2002; Nemoto et al. Pathobiology 65: 195, 1997; Logsdon et al., Cancer Res. 63: 2649, 2003), yet it is downregulated in melanoma tissue (Tasty et al, (1997)).

### BRIEF SUMMARY OF THE INVENTION

Disclosed are methods and compositions relating to inhibitors of EphA4 and EphA2. For example, disclosed herein is a method of treating a subject, the method comprising administering to the subject an EphA2/4 inhibitor. The subject can be suffering or be at risk of suffering nerve injury. The subject can be suffering or be at risk of suffering cancer. The subject can have cancer cells in which EphA2 is activated above a threshold level. The method can further comprise measuring EphA2 receptor activity in cancer cells prior to administering the EphA2/4 inhibitor. The subject can be suffering or be at risk of suffering tumor angiogenesis.

Also disclosed herein is a method of identifying compounds, the method comprising determining the binding characteristics of a test compound in the presence and absence of an EphA2/4 inhibitor, wherein if the test compound exhibits noncompetitive binding with the EphA2/4 inhibitor, then the test compound is identified as a noncompetitive binder of EphA2 and/or EphA4 (relative to the EphA2/4 inhibitor). The method can further comprise linking the noncompetitive binder to an EphA2/4 inhibitor via a linker to form a linked EphA2/4 binder. The method can further comprise administering to a subject the linked EphA2/4 binder. The subject can have suffered or is at risk of suffering nerve injury. The subject can be suffering or is at risk from suffering cancer. The subject can have cancer cells in which EphA2 is activated above a threshold level. The method can further comprise measuring EphA2 receptor activity in cancer cells prior to administering the EphA2/4 inhibitor. The subject can be suffering or is at risk of suffering tumor angiogenesis.

Also disclosed herein is a method of identifying compounds that interact with EphA4, the method comprising bringing into contact a test compound, an EphA2/4 inhibitor composition, and an EphA4 receptor, wherein the EphA2/4 inhibitor composition comprises an EphA2/4 inhibitor; and detecting unbound EphA2/4 inhibitor composition, wherein a given amount of unbound EphA2/4 inhibitor composition indicates a compound that interacts with EphA4. The EphA2/4 inhibitor composition can further comprise a moiety linked to the EphA2/4 inhibitor. The moiety linked to the EphA2/4 inhibitor can further comprise a detectable agent. The method can further comprise administering to a subject the test compound that interacts with EphA4. The subject in the disclosed method could have suffered or could be at risk of suffering nerve injury. The subject in the disclosed method can be suffering or is at risk of suffering from cancer. The subject in the disclosed method can have cancer cells in which EphA2 is activated above a threshold level. The method can further comprise measuring EphA2 receptor activity in cancer cells prior to administering the EphA2/4 inhibitor, and wherein in the subject can be suffering from or be at risk of suffering from tumor angiogenesis.

Also disclosed herein is a method of identifying a subject as having EphA4 receptor activity of interest, the method comprising measuring EphA4 receptor activity in the cell of a subject in the presence of an EphA2/4 inhibitor, wherein the subject has EphA4 receptor activity of interest if the measured EphA4 receptor activity differs from a reference EphA4 receptor activity by more than a threshold amount. The reference EphA4 receptor activity can be normal EphA4 receptor activity of a normal cell. The reference EphA4 receptor activity can be a non-pathological EphA4 receptor activity. The described method can be such that the measured EphA4 receptor activity is lower than the reference EphA4 receptor activity by more than the threshold amount.

Also disclosed herein is a method of identifying a subject as having EphA2 receptor activity of interest, the method comprising measuring EphA2 receptor activity in the cell of a subject in the presence of an EphA2/4 inhibitor, wherein the subject has EphA2 receptor activity of interest if the measured EphA2 receptor activity differs from a reference EphA2 receptor activity by more than a threshold amount. The reference EphA2 receptor activity can be normal EphA2 receptor activity of a normal cell. The reference EphA2 receptor activity can be a non-pathological EphA2 receptor activity. The described method can be such that the measured EphA2 receptor activity is lower than the reference EphA2 receptor activity by more than the threshold amount. The method for all the disclosed materials can further comprise administering to a subject to EphA2/4 inhibitor. The subject can have suffered or is at risk of suffering nerve injury. The subject can be suffering or is at risk from suffering cancer. The subject can have cancer cells in which EphA2 is activated above a threshold level. The method can further comprise measuring EphA2 receptor activity in cancer cells prior to administering the EphA2/4 inhibitor. The subject can be suffering or is at risk of suffering tumor angiogenesis. Also disclosed herein can be a pharmaceutical composition comprising an EphA2/4 inhibitor and a pharmaceutical acceptable carrier.

As an example, the EphA2/4 inhibitor can have the generic molecular structure of Formula I:

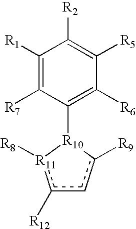

In Formula I, R₁ is R₃ or R₄ and R₂ is R₃ or R₄, wherein R₁ and R₂ are not both R₃ or both R₄; R₃ is —H, —OH, or —SH; R₄ is —COOH, —CH₂—COOH, or —CH₂—CH₂—COOH; R₅, R₆, and R₇ are each independently —H or —OH; R₁₀ is N or C and R₁₁ is O or C, wherein R₁₀ and R₁₁ are not both simultaneously C; wherein if R₁₀ is N, then R₈ and R₉ are each independently —CH₃, —CH₂—CH₃, or —CH₂—CH₂—CH₃, and R₁₁ is C; wherein if R₁₁ is O, then R₈ and R₉ are each H, and R₁₀ is C; R₁₂ is H, or

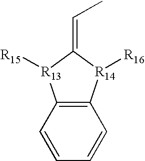

R₁₃ and R₁₄ are each C or S; R₁₅ and R₁₆ are each ═O or absent, wherein if R₁₃ is S, then R₁₅ is absent, wherein if R₁₃ is C, then R₁₅ is ═O, wherein if R₁₄ is S, then R₁₆ is absent, and wherein if R₁₄ is C, then R₁₆ is ═O.

R₁₂ can also be, —(CH₂)ₙ—CH₃ wherein n is an integer from 0 to 10, —O—(CH₂)ₙ—CH₃ wherein n is an integer from 0 to 10, —(O—CH₂)ₙ—CH₃ wherein n is an integer from 0 to 10, —(CH₂)ₙ—O—CH₃ wherein n is an integer from 1 to 10, —(CH₂)ₙ—CH═CH₂ wherein n is an integer from 1 to 10.

R₁₂ can also be a linker L, wherein L is, for example, —(CH₂)ₙ—R₁₇ wherein n is an integer from 1 to 10, —O—(CH₂)ₙ—R₁₇ where in n is an integer from 1 to 10, —(O—CH₂)ₙ—R₁₇ wherein n is an integer from 1 to 10, —(CH₂)ₙ—O—R₁₇ wherein n is an integer from 1 to 10, —(CH₂)ₙ—CH═R₁₈ wherein n is an integer from 1 to 10.

R₁₇ can be phenyl, biphenyl, naphtyl, tetrahydropyranyl (THP), trialkylsilyl wherein each alkyl chain has 1 to 3 carbons, dimethylsilyl, alicyclic cage group e.g. adamantly group or norbornyl group, alicyclic fused group e.g. naphtyl group, lactonyl group, saturated cyclic hydrocarbons, saturated polycyclic hydrocarbons, unsaturated cyclic hydrocarbons, unsaturated polycyclic hydrocarbons, pyrrolyl group, fluorenyl group, indan group, substituted indan group, indan-1,3-dione.

R₁₈ can be CH₂, saturated cyclic hydrocarbons, saturated polycyclic hydrocarbons, fluorenyl group, indan group, substituted indan group, and indan-1,3-dione.

As another example, the EphA2/4 inhibitor also can have the generic molecular structure of Formula II:

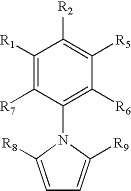

In Formula II, R₁ is R₃ or R₄; R₂ is R₃ or R₄, wherein R₁ and R₂ are not both R₃ or both R₄, wherein R₃ is —H, —OH, or —SH, wherein R₄ is —COOH, —CH₂—COOH, or —CH₂—CH₂—COOH; R₅, R₆, and R₇ are each independently —H or —OH; and R₈ and R₉ are each independently —CH₃, or —CH₂—CH₃, or —CH₂—CH₂—CH₃.

As another example, the EphA2/4 inhibitor can have the generic molecular structure of Formula III:

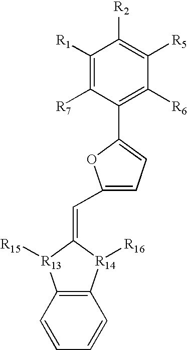

In Formula III, R₁ is R₃ or R₄; R₂ is R₃ or R₄, wherein R₁ and R₂ are not both R₃ or both R₄, wherein R₃ is —H, —OH, or —SH, wherein R₄ is —COOH, —CH₂—COOH, or —CH₂—CH₂—COOH; R₅, R₆, and R₇ are each independently —H or —OH; R₁₃ and R₁₄ are each C or S; R₁₅ and R₁₆ are each ═O or absent, wherein if R₁₃ is S, then R₁₅ is absent, wherein if R₁₃ is C, then R₁₅ is ═O, wherein if R₁₄ is S, then R₁₆ is absent, and wherein if R₁₄ is C, then R₁₆ is ═O.

The compounds of Formulas I and III can be further defined wherein if R₁₃ is S, then R₁₄ is C, and if R₁₄ is S, then R₁₃ is C or wherein R₁₃ and R₁₄ are each C and R₁₅ and R₁₆ are each ═O. The compounds of Formulas I, II and III also can be further defined wherein R₅ is —OH, R₆ is —H, and R₇ is —H; wherein R₅ is —H, R₆ is —H, and R₇ is —OH; or wherein R₅ is —H, R₆ is —H, and R₇ is —H. The compounds of Formulas I, II and III also can be further defined wherein R₈ is —CH₃ and R₉ is —CH₂—CH₃; wherein R₈ is —CH₂—CH₃ and R₉ is —CH₃; or wherein R₈ is —CH₃ and R₉ is —CH₃. The compounds of Formulas I, II and III also can be further defined wherein R₃ is —OH, and wherein R₄ is —COOH, —CH₂—COOH, or —CH₂—CH₂—COOH. The compounds of Formulas I, II and III also can be further defined wherein R₃ is —OH, and wherein R₄ is —COOH.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B. A. Results from the high throughput screen showing the ELISA plate from which compound 1 was identified (peak in middle of plate). Control wells containing EphA4 fused to alkaline phosphatase (AP) but no inhibitory peptide (front left and back right of plate); control wells containing AP instead of EphA4 AP (grey peaks at back left and middle right of plate); control wells containing only buffer (white peaks); wells containing a compound from the chemical library that inhibits binding of EphA4 AP to the KYL peptide (peak in middle of plate); and wells that do not contain inhibitory compounds (low peaks in middle of plate). B. 2,5-dimethylpyrrolyl benzene derivatives identified in the high throughput screening for EphA4 inhibitors. The first value in the % Inhibition column was obtained in the original screen with 10 μg/mL compound, the second value was obtained in a confirmatory repeat of the experiment. IC₅₀ values were calculated by measuring binding of EphA4 AP to immobilized KYL peptide or ephrin-A5 AP to immobilized EphA4 Fc in the presence of different concentrations of the compounds.

FIG. 2. Compound 1 and compound 2 inhibit EphA4 AP binding to immobilized biotinylated KYL peptide and ephrin-A5 AP binding to the immobilized EphA4 ectodomain fused to Fc in a concentration-dependent manner, as shown in the two top panels for each compound. The bottom left panels for each compound show the binding of ephrin-A5 AP to immobilized EphA4 Fc in the presence of different concentrations of each compound: () 0 μM (∘) 10 μM (▪) 20 μM (□) 30 μM (▴) 40 μM (Δ) 50 μM. These curves were used to calculate the dissociation constants (K_{D}) and maximal binding (B_{MAX}) used in the bottom right panels for each compound to determine Kᵢ values. Error bars indicate standard error from triplicate measurements.

FIGS. 3A and 3B. A. Ephrin-A5 AP binding to immobilized EphA receptor Fc fusion proteins and ephrin-B2 AP binding to immobilized EphB receptor Fc fusion proteins were measured in the presence of the indicated concentrations of compounds 1 and 2. The histogram shows the ratio of ephrin AP bound in the presence and in the absence of the compounds. Error bars indicate standard error from 3 measurements. B. IC₅₀ values for inhibition of EphA4 AP and EphA2 AP binding to the indicated immobilized ephrins by compounds 1 and 2.

FIG. 4. Structures of 2,5-dimethylpyrrolyl benzoic acid derivatives and related compounds that were examined are shown. IC₅₀ values (μM) for inhibition of EphA4 AP binding to the KYL peptide and ephrin-A5 AP binding to EphA4 Fc are shown below. The compounds are arranged in order of decreasing potency for inhibition of EphA4-ephrin-A5 binding or EphA4-KYL binding. Standard errors are shown for IC₅₀ values obtained from multiple experiments. Only compounds 1 through 4 were able to detectably inhibit EphA4-ephrin-A5 binding.

FIG. 5. Compounds related to compounds 1 and 2. Structures of 2,5-dimethylpyrrolyl benzoic acid derivatives and related compounds that were examined are shown. IC₅₀ values (μM) for inhibition of EphA4 AP binding to the KYL peptide and ephrin-A5 AP binding to EphA4 Fc are shown below each compound. The compounds are arranged in order of decreasing potency for inhibition of EphA4-ephrin-A5 binding or EphA4-KYL binding.

FIGS. 6A-6F. A. HT22 neuronal cells pretreated with the indicated concentrations of compounds 1 or 2 for 15 min were stimulated with 0.5 μg/mL ephrin-A5 Fc (+) or Fc as a control (−) for 20 min in the continued presence of the compounds. EphA4 immunoprecipitates were probed with anti-phosphotyrosine antibody (PTyr) and reprobed for EphA4. C indicates immunoprecipitations performed with control antibodies from non-immunized rabbits. B. The histogram shows the levels of EphA4 phosphorylation quantified from immunoblots and normalized to the amount of immunoprecipitated EphA4. Error bars represent the standard error from 4 experiments for compound 1 and 3 experiments for compound 2. Receptor phosphorylation levels were compared to those in ephrin-stimulated cells in the absence of compounds by one-way ANOVA and Dunnett's post test. *P<0.05; *P<0.01. C. COS7 cells pretreated with the indicated concentrations of compounds 1 or 2 for 15 min were stimulated with 0.1 μg/mL ephrin-A1 Fc or Fc as a control in the continued presence of the compounds. EphA2 immunoprecipitates were probed with anti-phosphotyrosine antibody (PTyr) and reprobed for EphA2. C indicates immunoprecipitations performed with control antibodies. D. The histogram shows the levels of EphA2 phosphorylation quantified from immunoblots and normalized to the amount of immunoprecipitated EphA2. Error bars represent the standard error from 2 experiments. Statistical analyses were performed as in (B). HUVE cells were left unstimulated or stimulated with TNFα for 2 hours in the presence of 400 μM compound 1. Duplicate untreated samples are shown. C indicates immunoprecipitations performed with control antibodies. EphA2 immunoprecipitates were probed with anti-phosphotyrosine antibody (PTyr) and reprobed for EphA2. Levels of EphA2 phosphorylation quantified from immunoblots and normalized to the amount of immunoprecipitated EphA2 were also performed. E. Receptor phosphorylation levels in cells treated with compound 1 were compared to those in non-treated samples by non-paired Student's t-test. *P<0.01. The same protocol described in (C) was used, except that COS7 cells were stimulated with 0.5 μg/mL of ephrin-B2 Fc and the EphB2 receptor was immunoprecipitated. F. COS7 cells pretreated with the indicated concentrations of compounds 1 or 2 were stimulated with EGF (+) or left unstimulated (−). Lysates were probed with anti-phosphotyrosine antibody (PTyr) and reprobed for the EGF receptor.

FIG. 7. HT22 neuronal cells were grown in the presence of the indicated concentrations of compounds 1 and 2 for 1, 2 and 3 days. Only DMSO was used in the “0 μM” sample, as a control. After addition of MTT, absorbance was measured at 570 nm to determine the levels of viable cells present. The histograms show the absorbance obtained for each condition normalized to the absorbance in the absence of the compounds. Error bars represent standard error from 3 measurements.

FIGS. 8A-8F. Compounds 1 and 2 block EphA4-dependent growth cone collapse in retinal neurons. A. Explants from embryonic day 6 chicken embryonic were preincubated with 5 μM KYL peptide for 15 min, stimulated for 30 min with 1 μg/mL ephrin-A5 Fc or Fc as a control in the continued presence of the peptide, and stained with rhodamine-phalloidin to label filamentous actin. B. Histogram showing the mean percentages of collapsed growth cones. Growth cones were scored as collapsed when no lamellipodia or filopodia were visible at the tip of the neurite. Approximately 30-200 growth cones per condition were scored in each experiment and error bars indicate standard error from 3 independent experiments. C-F. Experiments were performed as in (A), except that retinal explants were treated with 400 μM compound 1 (C,D) or compound 2 (E,F). Approximately 80-250 growth cones per condition were scored in each experiment, and error bars indicate standard error from 3 experiments. Collapsed growth cones under different conditions were compared to those in the Fc control condition by one-way ANOVA and Dunnett's post test. *P<0.05 and **P<0.01. Scale bars in (A), (C), (E)=25 μM.

FIGS. 9A-9G. A. PC3 cells pretreated for 15 min with the indicated concentrations of compound 1 or compound 2 were stimulated with 0.5 μg/mL ephrin-A1 Fc (+) or Fc as a control (−) for 20 min in the continued presence of the compounds. EphA2 immunoprecipitates were probed with anti-phosphotyrosine antibody (PTyr) and reprobed for EphA2. C indicates immunoprecipitations performed with control antibodies. B. Histogram showing the levels of EphA2 phosphorylation normalized to the amount of immunoprecipitated EphA2. Error bars indicate standard error from 3 experiments. Receptor phosphorylation levels were compared with those in the ephrin-stimulated cells by one-way ANOVA and Dunnett's post test. *P<0.05 and *P<0.01. C. PC3 cells stimulated with compound 1 as in (A) were stained with rhodamine-phalloidin to label actin filaments and DAPI to label nuclei. DMSO was used as a control (0 μM). D. Histogram showing the mean area of the cells normalized to the value obtained for the Fc stimulated cells. E. Histogram showing the mean percentage of retracting cells. Cells having rounded shape and area less than 20% of the mean value obtained for the Fc stimulated cells were scored as retracting. Error bars in D and E indicate standard error from 3 experiments. F-H. The same experiments as in (C-E) were performed using compound 2. The areas occupied by the cells and the percentage of cell retraction under different conditions were compared to those in the Fc control condition by one-way ANOVA and Dunnett's post test. *P<0.05 and **P<0.01. Scale bars in (C) and (F)=50 μm.

FIGS. 10A-10C. (a) Stereo view of the two disulfide bridges in the EphA4 ligand-binding domain built into the simulated annealing 2Fo-Fc electron density map contoured at 1.5a. (b) Ribbon representation of two EphA4 ligand-binding domain molecules A and B (Mol-A and Mol-B) in one asymmetric unit. The arrows are used to indicate the novel interface between the two molecules. (c) Ribbon representation of two EphA4 molecules in one asymmetric unit that have differential packing contacts with molecules in other asymmetric units.

FIGS. 11A and 11B. (a) Stereo view of the superimposition of the two EphA4 ligand-binding domain structures observed in the same asymmetric unit (structure A and structure B). (b) Stereo view of the superimposition of two EphA4 structures (structure A and structure B) with previously determined EphB2 and EphB4 structures.

FIGS. 12A-12B. (a) Superimposition of the ligand-binding domains of EphA4 Structure A (light grey) and EphA2 (3C8X; dark grey). (b) Superimposition of the ligand-binding domains of EphA4 Structure B (light grey) and EphA2 (dark grey). The green arrows are used to indicate the unique short 3₁₀-helix only presented in EphA receptors.

FIGS. 13A-13D. The ITC titration profiles of the binding reaction of the EphA4 ligand-binding domains with compound 1: (a); and with compound 2: (c), Integrated values for reaction heats with subtraction of the corresponding blank results normalized by the amount of ligand injected versus molar ratio of compound 1/EphA4 (b) and of compound 2/EphA4 (d) The detailed conditions and setting of the ITC experiments are presented in Materials and Methods as well as Table 2.

FIGS. 14A-14D. (a) Far-UV circular dichroism spectra of the EphA4 ligand-binding domain in the absence (black) and in the presence of compound 1 (dark grey) or compound 2 (light grey). The chemical structures of the two compounds are presented. (b) ¹H-¹⁵N NMR HSQC spectra of the EphA4 ligand-binding domain in the absence (dark grey) and in the presence of compound 1 (light grey). (c) Residue-specific chemical shift differences (CSD) of the EphA4 ligand-binding domain in the presence of compound 1. (d) Residue-specific chemical shift differences (CSD) of the EphA4 ligand-binding domain in the presence of compound 2. Labeled bars indicate residues with CSD larger than 2.5 times of the standard deviation as described in Materials and Methods. In all experiments the molar ratio of EphA4 to compound was 1:6.

FIGS. 15A-15B. (a) Stereo view of the superimposition of the unbound EphA4 Structure A with its 3 selected docking models in complex with compound 1. (b) Stereo view of the superimposition of the unbound EphA4 Structure A with its 3 selected docking models in complex with compound 2. Both sticks and dots are used to highlight residues Ile31-Met32 in the D-E loop, Gln43 in the E β-strand and Ile131-Gly132 in the J-K loop.

FIGS. 16A-16B. Models of Structure B in complex with small molecule antagonists. (a) Stereo view of the superimposition of the unbound EphA4 Structure B with its 3 selected docking models in complex with compound 1. (b) Stereo view of the superimposition of the unbound EphA4 Structure B with its 3 selected docking models in complex with compound 2. Both sticks and dots are used to highlight residues Ile31-Met32 in the D-E loop, Gln43 in the E β-strand and Ile 131-Gly 132 in the J-K loop.

FIGS. 17A-17D. Surface representation of the EphA4 binding cavity of the docking model with the lowest energy. (a) EphA4 Structure A with compound 1; (b) EphA4 Structure A with compound 2; (c) EphA4 Structure B with compound 1; and (d) EphA4 Structure B with compound 2. The small molecule antagonists are represented by sticks. EphA4 residues Ile31-Met32 in the D-E loop are to the left ((a) and (b)) or below ((c) and (d)) the antagonists, residue Gln43 in the E β-strand is the dark area below ((a) and (b)) or below and right ((c) and (d)) of the antagonists and residues Ile131-Gly132 in the J-K loop are above the antagonists.

FIGS. 18A-18B. (a) Stereo view of the superimposition of four selected EphA4-small molecule models with previously-determined structures of EphB-ephrin complexes (1KGY, 1SHW and 2HLE). EphA4 is represented by a light grey ribbon and the small molecules by the grey cloud. The EphB receptors are medium grey; and ephrin-B2/ephrin-A5 are dark grey. The arrows indicate the contact regions outside of the ligand-binding channel that contribute to the high affinity Eph. Receptor-ephrin binding interface. (b) Stereo view of the superimposition of four selected EphA4-small molecule models with previously determined structures of EphB-peptide complexes (2QBX and 2BBA). EphA4 is light grey, EphB receptors are medium grey, one peptide is dark grey. The arrow indicates a conserved binding motif identified in all the EphB structures in complex with either ephrins or antagonistic peptides (Chrencik et al. (2007) J Biol. Chem. 282, 36505-13).

### DETAILED DESCRIPTION OF THE INVENTION

The disclosed method and compositions may be understood more readily by reference to the following detailed description of particular embodiments and the Example included therein and to the Figures and their previous and following description.

The Eph receptors are subdivided in two classes, which in the human genome include 9 EphA receptors, which preferentially bind the 5 ephrin-A ligands, and 5 EphB receptors, which preferentially bind the 3 ephrin-B ligands. Binding between receptors and ephrins of the same class is highly promiscuous, and few examples of inter-class binding have also been reported (Pasquale, (2004) Nat Neurosci 7(5), 417-418; Klein, R. (2004) Curr Opin Cell Biol 16, 580-589; Yamaguchi and Pasquale, (2004) Curr Opin Neurobiol 14, 288-29). The Eph receptors exert their effects by interacting with ligands, the ephrins, which are also membrane-bound proteins. Eph receptor-ephrin interaction is mediated by two binding sites in the amino-terminal ephrin-binding domain of the receptor: a high affinity site, which includes a hydrophobic cavity that accommodates a protruding loop of the ephrin (the G-H loop), and a separate low affinity site (Himanen et al. (2007) Curr Opin Cell Biol 19(5), 534-542). A third molecular interface located in the adjacent cysteine-rich region of the receptor has also been described (Smith et al. (2004) J Biol Chem 279(10), 9522-9531). Despite the presence of several binding interfaces, peptides that target the high affinity site are sufficient to inhibit Eph receptor-ephrin binding (Koolpe et al. (2005) J Biol Chem 280(17), 17301-17311, Chrencik et al. (2006) Structure 14(2), 321-330, Chrencik et al. (2007) J Biol Chem 282(50), 36505-36513). Interestingly, unlike the ephrins whose binding is highly promiscuous, a number of the peptides that were identified by phage display selectively bind to only one or a few of the Eph receptors (Koolpe et al. (2005) J Biol Chem 280(17), 17301-17311, Koolpe et al. (2002) J Biol Chem 277(49), 46974-46979, Murai et al. (2003) Mol Cell Neurosci 24(4), 1000-1011). Other substances that modulate Eph-ephrin interactions have also been identified, including antibodies and soluble forms of Eph receptors and ephrins extracellular domains (Pasquale, (2005) Nat Rev Mol Cell Biol 6(6), 462-475, Ireton and Chen, (2005) Curr Cancer Drug Targets 5(3), 149-157, Noren and Pasquale, (2007) Cancer Res 67(9), 3994-3997, Wimmer-Kleikamp and Lackmann, (2005) IUBMB Life 57(6), 421-431). Several small molecule inhibitors of the Eph receptor kinase domain have also been reported (Caligiuri et al. (2006) Chem Biol 13(7), 711-722, Karaman et al. (2008) Nat Biotechnol 26(1), 127-132, Miyazaki et al. (2008) Bioorganic & medicinal chemistry letters 18(6), 1967-1971, Kolb et al. (2008) Proteins). These inhibitors occupy the ATP binding pocket of the receptors and are usually broad-specificity inhibitors that target different families of tyrosine kinases (Caligiuri et al. (2006) Chem Biol 13(7), 711-722, Karaman et al. (2008) Nat Biotechnol 26(1), 127-132). Epigallocatechin gallate, a green tea derivative known to inhibit several tyrosine kinases, has also been shown to inhibit EphA receptor-mediated human umbilical vein endothelial cell migration and capillary-like tube formation, but the mechanism of action of this molecule has not been elucidated (Tang et al. (2007) J Nutr Biochem 18(6), 391-399). Although the size, polarity and geometry of the high affinity ephrin-binding pocket of the Eph receptors indicate that it might accommodate the tight binding of small molecular weight chemical compounds (Fry and Vassilev, (2005) J Mol Med 83(12), 955-963), no such inhibitors have been identified previously for any of the Eph receptors.

EphA4 has important functions in the developing and adult nervous system and is expressed in brain regions characterized by extensive synaptic remodeling (Bourgin et al. (2007) J. Cell Biol. 178, 1295-307; Richter et al. (2007) J. Neurosci. 27, 14205-15). In the adult, EphA4 is particularly enriched in the hippocampus and cortex, two brain structures important for learning and memory processes. While EphA4 interacts with ephrin-A ligands to mediate a variety of critical biological processes, such as inhibiting integrin downstream signaling pathways (Bourgin et al. (2007) J. Cell Biol. 178, 1295-307) and modulating sensory and motor projections (Gallarda et al. (2008) Science. 320, 233-6), this receptor is also able to bind all three ephrin-B ligands. For example, EphA4 interacts with ephrin-B1 expressed in human platelets to stabilize blood clot formation through an integrin-dependent mechanism (Prevost et al. (2005) Proc Natl Acad Sci U S A 102, 9820-9825). By interacting with ephrin-B2 and/or ephrin-B3, EphA4 regulates neuronal circuits important for coordinated movement and can inhibit the regeneration of injured spinal cord axons (Goldshmit et al. (2004) J Neurosci 24, 10064-10073; Benson et al. (2005) Proc Natl Acad Sci USA. 102, 10694-9; Du et al. (2007) Current pharmaceutical design 13, 2507-2518). Being the most promiscuous member of the Eph family, it was realized that EphA4 is particularly interesting to target. Furthermore, besides being a well know regulator of neural connectivity during development and of synaptic function in the adult brain (Klein (2004) Curr Opin Cell Biol 16(5), 580-589, Yamaguchi and Pasquale, (2004) Curr Opin Neurobiol 14(3), 288-296), EphA4 has also been linked to several pathologies. It was realized that this receptor is a good target for drug development. For example, the fact that EphA4 has been implicated in the inhibition of spinal cord regeneration after injury, by promoting the formation of the glial scar and inhibiting axon regrowth (Goldshmit et al. (2004) J Neurosci 24(45), 10064-10073, Fabes et al. (2006) Eur J Neurosci 23(7), 1721-1730, Fabes et al. (2007) Eur J Neurosci 26(9), 2496-2505) stimulated the discovery described herein that EphA4 inhibitors can have beneficial effects on damaged neural tissue. In addition, EphA4 is expressed on the surface of human platelets, where it promotes thrombus stabilization (Prevost et al. (2005) Proc Natl Acad Sci USA 102(28), 9820-9825). EphA4 has also been detected in several different types of cancer cells (Easty et al. (1997) Int J Cancer 71(6), 1061-1065, Ashida et al. (2004) Cancer Res 64(17), 5963-5972, Iiizumi et al. (2006) Cancer Sci 97(11), 1211-1216) as well as in tumor endothelial cells (Yao et al. (2005) Am J Pathol 166(2), 625-636, Yamashita et al. (2008) J Biol Chem).

The critical roles of EphA4 in various physiological and pathological processes validate the discovery that this receptor can be a target for small molecule drugs to treat human diseases, such as spinal cord injury, abnormal blood clotting, and certain types of cancer (Prevost et al. (2005) Proc Natl Acad Sci USA 102, 9820-9825, Goldshmit et al. (2004) J Neurosci 24, 10064-10073; Benson et al. (2005) Proc Natl Acad Sci USA. 102, 10694-9; Du et al. (2007) Current pharmaceutical design 13, 2507-2518; Easty et al. (1997) Int J Cancer 71, 1061-1065; Ashida et al. (2004) Cancer Res 64, 5963-5972; Iiizumi et al. (2006) Cancer Sci 97, 1211-1216; Yao et al. (2005) Am J Pathol 166, 625-636). Prior to the present disclosure and despite intensive efforts, only several small molecule inhibitors of Eph receptors have been previously reported, all of which target the ATP binding site in the receptor cytoplasmic kinase domain (Caligiuri et al. (2006) Chem Biol 13, 711-722; Karaman et al. (2008) Nat Biotechnol 26, 127-132; Miyazaki et al. (2008) Bioorganic & medicinal chemistry letters 18, 1967-1971; Kolb et al. (2008) Proteins (10.1002/prot.22028). However, these molecules also inhibit the activities of other families of kinases (Caligiuri et al. (2006) Chem Biol 13, 711-722; Karaman et al. (2008) Nat Biotechnol 26, 127-132). Thus, these prior molecules are not EphA2/4 inhibitors.

As described herein, the high-affinity ephrin binding pocket of the Eph receptors has been discovered to be an attractive target for design of small molecules capable of inhibiting the Eph receptor signaling by blocking ephrin binding. A high throughput screen was performed to search for small molecules that can inhibit ligand binding to the extracellular domain of the EphA4 receptor. The exemplary base structure 2,5-dimethylpyrrolyl benzoic acid and its derivatives were discovered to inhibit the interaction of EphA4 with a peptide ligand as well as the natural ephrin ligands. Evaluation of a series of analogs identified an isomer with similar inhibitory properties and other less potent compounds. The discovered compounds act as competitive inhibitors, indicating that they target the high affinity ligand-binding pocket of EphA4. Two exemplary compounds inhibit ephrin-A5 binding to EphA4 with Ki values of 7 and 9 μM in ELISA assays (see Example 1). Surprisingly, despite the ability of each ephrin ligand to promiscuously bind many Eph receptors, the two exemplary compounds selectively target only EphA4 and the closely related EphA2 receptor Inhibitors selective for EphA4 and EphA2 receptors are referred to herein as “EphA2/4 inhibitors.” The compounds can also inhibit ephrin-induced phosphorylation of EphA4 and EphA2 in cells, without affecting cell viability or the phosphorylation of other receptor tyrosine kinases. Furthermore, the compounds inhibit EphA4-mediated growth cone collapse in retinal explants and EphA2-dependent retraction of the cell periphery in prostate cancer cells. These data demonstrate that the Eph receptor-ephrin interface can be targeted by inhibitory small molecules and indicate that the disclosed EphA2/4 inhibitors will be useful to discriminate the activities of EphA4 and EphA2 from those of other co-expressed Eph receptors that are activated by the same ephrin ligands. Furthermore, the disclosed inhibitors can be used to treat pathologies in which EphA4 and EphA2 are involved, including nerve injuries and cancer.

Also described herein is structural insight into the binding interactions between two exemplary EphA2/4 inhibitors and the EphA4 ligand-binding domain (see Example 2). Prior to the discoveries described herein, no structure has been published for the ligand-binding domain of any EphA subclass member. Disclosed herein is the crystal structure of the EphA4 ligand-binding domain and characterization of its binding to two exemplary antagonistic small molecules, namely 4- and 5-(2,5 dimethyl-pyrrol-1-yl)-2-hydroxy-benzoic acid by using isothermal titration calorimetry (ITC), circular dichroism (CD), nuclear magnetic resonance (NMR) spectroscopy and computational docking. The crystal structure of the EphA4 ligand-binding domain, which adopts the same jellyroll β-sandwich architecture as previously shown for EphB2 and EphB4. The similarity with EphB receptors is high in the core β-stranded regions, whereas large variations exist in the loops, particularly the D-E and J-K loops, which form the high-affinity ephrin binding channel. Isothermal titration calorimetry, NMR spectroscopy and computational docking was used to characterize the binding to EphA4 of two exemplary compounds, 4- and 5-(2,5 dimethyl-pyrrol-1-yl)-2-hydroxy-benzoic acid which antagonize ephrin-induced effects in EphA4-expressing cells. It was shown that the two exemplary compounds bind to the EphA4 ligand-binding domain with Kd values of 20.4 and 26.4 μM, respectively. NMR HSQC titrations revealed that upon binding, both molecules significantly perturb EphA4 residues Ile31-Met32 in the D-E loop, Gln43 in the E β-strand and Ile131-Gly132 in the J-K loop. Molecular docking shows that the molecules can occupy a cavity in the high-affinity ephrin binding channel of EphA4 in a similar manner, by interacting mainly with the EphA4 residues in the E strand, D-E and J-K loops. However, many of the interactions observed in Eph receptor-ephrin complexes are absent, which is consistent with the small size of the two molecules and may account for their relatively weak binding affinity. Thus, these results provide the first published structure of the ligand binding domain of an EphA receptor of the A subclass. Furthermore, the results demonstrate that the high-affinity ephrin-binding channel of the Eph receptors is amenable to targeting with small molecule antagonists and indicate avenues for further optimization.

Disclosed are methods and compositions relating to inhibitors of EphA4 and EphA2. For example, disclosed herein is a method of treating a subject, the method comprising administering to the subject an EphA2/4 inhibitor. The subject can be suffering or be at risk of suffering nerve injury. The subject can be suffering or be at risk of suffering cancer. The subject can have cancer cells in which EphA2 is activated above a threshold level. The method can further comprise measuring EphA2 receptor activity in cancer cells prior to administering the EphA2/4 inhibitor. The subject can be suffering or be at risk of suffering tumor angiogenesis.

Also disclosed herein is a method of identifying compounds, the method comprising determining the binding characteristics of a test compound in the presence and absence of an EphA2/4 inhibitor, wherein if the test compound exhibits noncompetitive binding with the EphA2/4 inhibitor, then the test compound is identified as a noncompetitive binder of EphA2 and/or EphA4 (relative to the EphA2/4 inhibitor). The method can further comprise linking the noncompetitive binder to an EphA2/4 inhibitor via a linker to form a linked EphA2/4 binder. The method can further comprise administering to a subject the linked EphA2/4 binder. The subject can have suffered or is at risk of suffering nerve injury. The subject can be suffering or is at risk from suffering cancer. The subject can have cancer cells in which EphA2 is activated above a threshold level. The method can further comprise measuring EphA2 receptor activity in cancer cells prior to administering the EphA2/4 inhibitor. The subject can be suffering or is at risk of suffering tumor angiogenesis.

Also disclosed herein is a method of identifying compounds that interact with EphA4, the method comprising bringing into contact a test compound, an EphA2/4 inhibitor composition, and an EphA4 receptor, wherein the EphA2/4 inhibitor composition comprises an EphA2/4 inhibitor; and detecting unbound EphA2/4 inhibitor composition, wherein a given amount of unbound EphA2/4 inhibitor composition indicates a compound that interacts with EphA4. The ability of the test compound to bind the EphA4 receptor and displace or prevent binding by the EphA2/4 inhibitor used in the method increases the amount of unbound EphA2/4 inhibitor. Thus, the increased amount of unbound EphA2/4 inhibitor indicates that the test compound interacts with the EphA4 receptor.

The EphA2/4 inhibitor composition can further comprise a moiety linked to the EphA2/4 inhibitor. The moiety linked to the EphA2/4 inhibitor can further comprise a detectable agent. The method can further comprise administering to a subject the test compound that interacts with EphA4. The subject in the disclosed method could have suffered or could be at risk of suffering nerve injury. The subject in the disclosed method can be suffering or is at risk of suffering from cancer. The subject in the disclosed method can have cancer cells in which EphA2 is activated above a threshold level. The method can further comprise measuring EphA2 receptor activity in cancer cells prior to administering the EphA2/4 inhibitor, and wherein in the subject can be suffering from or be at risk of suffering from tumor angiogenesis.

Also described herein is a method of identifying a subject as having EphA4 receptor activity of interest, the method comprising measuring EphA4 receptor activity in the cell of a subject in the presence of an EphA2/4 inhibitor, wherein the subject has EphA4 receptor activity of interest if the measured EphA4 receptor activity differs from a reference EphA4 receptor activity by more than a threshold amount. The reference EphA4 receptor activity can be normal EphA4 receptor activity of a normal cell. The reference EphA4 receptor activity can be a non-pathological EphA4 receptor activity. The described method can be such that the measured EphA4 receptor activity is lower than the reference EphA4 receptor activity by more than the threshold amount.

Also disclosed herein is a method of identifying a subject as having EphA2 receptor activity of interest, the method comprising measuring EphA2 receptor activity in the cell of a subject in the presence of an EphA2/4 inhibitor, wherein the subject has EphA2 receptor activity of interest if the measured EphA2 receptor activity differs from a reference EphA2 receptor activity by more than a threshold amount. The reference EphA2 receptor activity can be normal EphA2 receptor activity of a normal cell. The reference EphA2 receptor activity can be a non-pathological EphA2 receptor activity. The described method can be such that the measured EphA2 receptor activity is lower than the reference EphA2 receptor activity by more than the threshold amount. The method for all the disclosed materials can further comprise administering to a subject to EphA2/4 inhibitor. The subject can have suffered or is at risk of suffering nerve injury. The subject can be suffering or is at risk from suffering cancer. The subject can have cancer cells in which EphA2 is activated above a threshold level. The method can further comprise measuring EphA2 receptor activity in cancer cells prior to administering the EphA2/4 inhibitor. The subject can be suffering or is at risk of suffering tumor angiogenesis. Also disclosed herein can be a pharmaceutical composition comprising an EphA2/4 inhibitor and a pharmaceutical acceptable carrier.

As an example, the EphA2/4 inhibitor can have the generic molecular structure of Formula I:

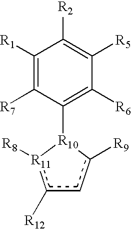

In Formula I, R₁ is R₃ or R₄ and R₂ is R₃ or R₄, wherein R₁ and R₂ are not both R₃ or both R₄; R₃ is —H, —OH, or —SH; R₄ is —COOH, —CH₂—COOH, or —CH₂—CH₂—COOH; R₅, R₆, and R₇ are each independently —H or —OH; R₁₀ is N or C and R₁₁ is O or C, wherein R₁₀ and R₁₁ are not both simultaneously C; wherein if R₁₀ is N, then R₈ and R₉ are each independently —CH₃, —CH₂—CH₃, or —CH₂—CH₂—CH₃, and R₁₁ is C; wherein if R₁₁ is O, then R₈ and R₉ are each H, and R₁₀ is C; R₁₂ is H, or

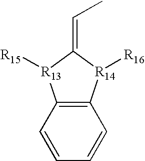

R₁₃ and R₁₄ are each C or S; R₁₅ and R₁₆ are each ═O or absent, wherein if R₁₃ is S, then R₁₅ is absent, wherein if R₁₃ is C, then R₁₅ is ═O, wherein if R₁₄ is S, then R₁₆ is absent, and wherein if R₁₄ is C, then R₁₆ is ═O.

As another example, the EphA2/4 inhibitor also can have the generic molecular structure of Formula II:

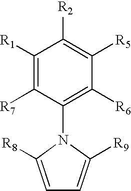

In Formula II, R₁ is R₃ or R₄; R₂ is R₃ or R₄, wherein R₁ and R₂ are not both R₃ or both R₄, wherein R₃ is —H, —OH, or —SH, wherein R₄ is —COOH, —CH₂—COOH, or —CH₂—CH₂—COOH; R₅, R₆, and R₇ are each independently —H or —OH; and R₈ and R₉ are each independently —CH₃, or —CH₂—CH₃, or —CH₂—CH₂—CH₃.

As another example, the EphA2/4 inhibitor can have the generic molecular structure of Formula III:

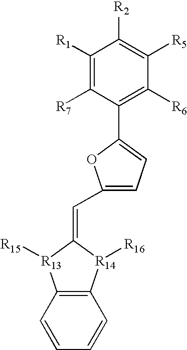

In Formula III, R₁ is R₃ or R₄; R₂ is R₃ or R₄, wherein R₁ and R₂ are not both R₃ or both R₄, wherein R₃ is —H, —OH, or —SH, wherein R₄ is —COOH, —CH₂—COOH, or —CH₂—CH₂—COOH; R₅, R₆, and R₇ are each independently —H or —OH; R₁₃ and R₁₄ are each C or S; R₁₅ and R₁₆ are each ═O or absent, wherein if R₁₃ is S, then R₁₅ is absent, wherein if R₁₃ is C, then R₁₅ is ═O, wherein if R₁₄ is S, then R₁₆ is absent, and wherein if R₁₄ is C, then R₁₆ is ═O.

The compounds of Formulas I and III can be further defined wherein if R₁₃ is S, then R₁₄ is C, and if R₁₄ is S, then R₁₃ is C or wherein R₁₃ and R₁₄ are each C and R₁₅ and R₁₆ are each ═O. The compounds of Formulas I, II and III also can be further defined wherein R₅ is —OH, R₆ is —H, and R₇ is —H; wherein R₅ is —H, R₆ is —H, and R₇ is —OH; or wherein R₅ is —H, R₆ is —H, and R₇ is —H. The compounds of Formulas I, II and III also can be further defined wherein R₈ is —CH₃ and R₉ is —CH₂—CH₃; wherein R₈ is —CH₂—CH₃ and R₉ is —CH₃; or wherein R₈ is —CH₃ and R₉ is —CH₃. The compounds of Formulas I, II and III also can be further defined wherein R₃ is —OH, and wherein R₄ is —COOH, —CH₂—COOH, or —CH₂—CH₂—COOH. The compounds of Formulas I, II and III also can be further defined wherein R₃ is —OH, and wherein R₄ is —COOH.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an EphA2/4 inhibitor is disclosed and discussed and a number of modifications that can be made to a number of molecules including the EphA2/4 inhibitor are discussed, each and every combination and permutation of EphA2/4 inhibitor and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

By “pharmaceutically acceptable” is meant a material that is not biologically, clinically or otherwise undesirable, i.e., the material can be administered to an individual along with the relevant active compound without causing clinically unacceptable biological effects or interacting in a deleterious manner with any of the other components of the pharmaceutical composition in which it is contained.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed, then “less than or equal to” the value, “greater than or equal to the value,” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The term “organic radical” defines a carbon containing moiety that forms a portion of a larger molecule, i.e. a moiety comprising at least one carbon atom, and can also often contain hydrogen atoms. Examples of organic radicals that comprises no heteroatoms are alkyls such as methyl, ethyl, n-propyl, or isopropyl moieties, or cyclic organic radicals such as phenyl or tolyl moieties, or 5,6,7,8-tetrahydro-2-naphthyl moieties. Organic radicals can and often do, however, optionally contain various heteroatoms such as halogens, oxygen, nitrogen, sulfur, phosphorus, or the like. Examples of organic residues include alkoxy or substituted alkoxy moieties such as methoxyl moieties or hydroxymethyl moieties, or in other examples trifluoromethyl moieties, mono or di-methyl amino moieties, carboxy moieties, formyl moieties, amide moieties, etc. An organic radical can have, for example, 1-26 carbon atoms, 1-18 carbon atoms, 1-12 carbon atoms, 1-8 carbon atoms, or 1-4 carbon atoms. Organic radicals often have a hydrogen bound to at least some of the carbon atoms of the organic radical. In some embodiments, an organic radical can contain 1-10, or 1-5 heteroatoms bound thereto

The term “alkyl” denotes a hydrocarbon group or residue which is structurally similar to an alkane compound modified by the removal of one hydrogen from the non-cyclic alkane and the substitution therefore of a non-hydrogen moiety. “Normal” or “Branched” alkyls comprise a non-cyclic, saturated, straight or branched chain hydrocarbon moiety having from 1 to 12 carbons, or 1 to 8 carbons, 1 to 6, or 1 to 4 carbon atoms. Examples of such alkyl radicals include methyl, ethyl, n-propyl, iso-propyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

The term “substituted alkyl” denotes an alkyl radical analogous to the above definition that is further substituted with one, two, or more additional organic or inorganic substituent groups. Suitable substituent groups include but are not limited to hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When more than one substituent group is present then they can be the same or different. The organic substituent moieties can comprise from 1 to 12 carbon atoms, or from 1 to 6 carbon atoms, or from 1 to 4 carbon atoms.

The term “alkenyl” denotes an alkyl residue as defined above that also comprises at least one carbon-carbon double bond. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

The term “substituted alkenyl” denotes an alkenyl residue, as defined above that is substituted with one or more additional moieties, but preferably one, two or three groups, selected from halogen, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “alkynyl” denotes a residue as defined above that comprises at least one carbon-carbon double bond. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “cycloalkyl” denotes a hydrocarbon group or residue which is structurally similar to a cyclic alkane compound modified by the removal of one hydrogen from the cyclic alkane and substitution therefore of a non-hydrogen moiety. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

The term “substituted cycloalkyl” denotes a cycloalkyl residue as defined above that is further substituted with one, two, or more additional organic or inorganic groups that can include but are not limited to halogen, alkyl, substituted alkyl, hydroxyl, alkoxy, substituted alkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

The term “cycloalkenyl” denotes a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like. The term “substituted cycloalkenyl” denotes a cycloalkyl as defined above further substituted with one or more groups selected from halogen, alkyl, hydroxyl, alkoxy, substituted alkoxy, haloalkoxy, carboxy, carboalkoxy, alkylcarboxamido, substituted alkylcarboxamido, dialkylcarboxamido, substituted dialkylcarboxamido, amino, mono-substituted amino or di-substituted amino. When the cycloalkenyl is substituted with more than one group, they can be the same or different. The organic substituent groups can comprise from 1 to 12 carbon atoms, or from 1 to 6 carbon atoms, or from 1 to 4 carbon atoms.

The term “alkoxy” as used herein denotes an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like.

The term “substituted alkoxy” denotes an alkoxy residue of the above definition that is substituted with one or more substituent groups, but preferably one or two groups, which include but are not limited to hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy. When more than one group is present then they can be the same or different. The organic substituent groups can comprise from 1 to 12 carbon atoms, or from 1 to 6 carbon atoms, or from 1 to 4 carbon atoms.

The term “mono-substituted amino” denotes a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyethylamino (—NH—CH₂CH₂OH), and the like.

The term “di-substituted amino” denotes a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

The term “haloalkyl” denotes an alkyl residue as defined above, substituted with one or more halogens, preferably fluorine, such as a trifluoromethyl, pentafluoroethyl and the like.

The term “haloalkoxy” denotes a haloalkyl residue as defined above that is directly attached to an oxygen to form trifluoromethoxy, pentafluoroethoxy and the like.

The term “acyl” denotes a R—C(O)— residue having an R group containing 1 to 8 carbons. Examples include but are not limited to formyl, acetyl, propionyl, butanoyl, iso-butanoyl, pentanoyl, hexanoyl, heptanoyl, benzoyl and the like, and natural or un-natural amino acids.

The term “acyloxy” denotes an acyl radical as defined above directly attached to an oxygen to form an R—C(O)O— residue. Examples include but are not limited to acetyloxy, propionyloxy, butanoyloxy, iso-butanoyloxy, benzoyloxy and the like.

The term “aryl” denotes a ring radical containing 6 to 18 carbons, or preferably 6 to 12 carbons, comprising at least one six-membered aromatic “benzene” residue therein. Examples of such aryl radicals include phenyl, naphthyl, and ischroman radicals. The term “substituted aryl” denotes an aryl ring radical as defined above that is substituted with one or more, preferably 1, 2, or 3 organic or inorganic substituent groups, which include but are not limited to a halogen, alkyl, substituted alkyl, hydroxyl, cycloalkyl, amino, mono-substituted amino, di-substituted amino, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, substituted heterocyclic ring wherein the terms are defined herein. The organic substituent groups can comprise from 1 to 12 carbon atoms, or from 1 to 6 carbon atoms, or from 1 to 4 carbon atoms.

The term “heteroaryl” denotes an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

The term “halo” or “halogen” refers to a fluoro, chloro, bromo or iodo group.

For the purposes of the present disclosure the terms “compound,” “analog,” and “composition of matter” stand equally well for the chemical entities described herein, including all enantiomeric forms, diastereomeric forms, salts, and the like, and the terms “compound,” “analog,” and “composition of matter” are used interchangeably throughout the present specification.

### A. EphA2/4 Inhibitor

Disclosed are compounds and compositions comprising EphA2/4 inhibitors. As used herein, a “EphA4-specific inhibitor” is a molecule or compound that inhibits EphA4 receptor activity with a Ki more than five times lower than the Ki of the compound for any other Eph receptor except EphA2 receptor. As used herein, a “EphA2-specific inhibitor” is a molecule or compound that inhibits EphA2 receptor activity with a Ki more than five times lower than the Ki of the compound for any other Eph receptor except EphA4 receptor. As used herein, a “EphA2/4 inhibitor” is an EphA4-specific inhibitor, an EphA2-specific inhibitor, or both an EphA4-specific inhibitor and an EphA2-specific inhibitor. Due to the relationship of the binding pockets of EphA4 and EphA2 as described herein, an EphA2/4 inhibitor can, but need not, inhibit both EphA4 and EphA2. Similarly, an EphA4- or EphA2-specific inhibitor can, but need not, inhibit both EphA4 and EphA2. Thus, the disclosed EphA2/4 inhibitors do not include all inhibitors of EphA4 receptor activity but rather are those EphA4 inhibitors that differentially inhibit EphA4 and/or EphA2. EphA2/4 inhibitors can also be referred to herein as “Eph-specific inhibitors.”

Disclosed herein are methods for confirming and analyzing the inhibition of EphA4 and EphA2 by the disclosed compounds and compositions, as well as methods for identifying additional EphA2/4 inhibitors.

As an example, the EphA2/4 inhibitor can have the generic molecular structure of Formula I:

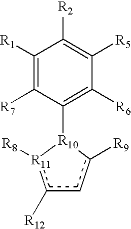

In Formula I, R₁ is R₃ or R₄ and R₂ is R₃ or R₄, wherein R₁ and R₂ are not both R₃ or both R₄; R₃ is —H, —OH, or —SH; R₄ is —COOH, —CH₂—COOH, or —CH₂—CH₂—COOH; R₅, R₆, and R₇ are each independently —H or —OH; R₁₀ is N or C and R₁₁ is O or C, wherein R₁₀ and R₁₁ are not both simultaneously C; wherein if R₁₀ is N, then R₈ and R₉ are each independently —CH₃, —CH₂—CH₃, or —CH₂—CH₂—CH₃, and R₁₁ is C; wherein if R₁₁ is O, then R₈ and R₉ are each H, and R₁₀ is C; R₁₂ is H, or

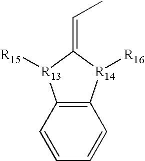

R₁₃ and R₁₄ are each C or S; R₁₅ and R₁₆ are each ═O or absent, wherein if R₁₃ is S, then R₁₅ is absent, wherein if R₁₃ is C, then R₁₅ is ═O, wherein if R₁₄ is S, then R₁₆ is absent, and wherein if R₁₄ is C, then R₁₆ is ═O.

R₁₂ can also be, —(CH₂)ₙ—CH₃ wherein n is an integer from 0 to 10, —O—(CH₂)ₙ—CH₃ wherein n is an integer from 0 to 10, —(O—CH₂)ₙ—CH₃ wherein n is an integer from 0 to 10, —(CH₂)ₙ—O—CH₃ wherein n is an integer from 1 to 10, —(CH₂)ₙ—CH═CH₂ wherein n is an integer from 1 to 10.

R₁₂ can also be a linker L, wherein L, for example, is —(CH₂)ₙ—R₁₇ wherein n is an integer from 1 to 10, —O—(CH₂)ₙ—R₁₇ where in n is an integer from 1 to 10, —(O—CH₂)ₙ—R₁₇ wherein n is an integer from 1 to 10, —(CH₂)ₙ—O—R₁₇ wherein n is an integer from 1 to 10, —(CH₂)ₙ—CH═R₁₈ wherein n is an integer from 1 to 10.

R₁₇ can be phenyl, biphenyl, naphtyl, tetrahydropyranyl (THP), trialkylsilyl wherein each alkyl chain has 1 to 3 carbons, dimethylsilyl, alicyclic cage group e.g. adamantly group or norbornyl group, alicyclic fused group e.g. naphtyl group, lactonyl group, saturated cyclic hydrocarbons, saturated polycyclic hydrocarbons, unsaturated cyclic hydrocarbons, unsaturated polycyclic hydrocarbons, pyrrolyl group, fluorenyl group, indan group, substituted indan group, indan-1,3-dione.

R₁₈ can be CH₂, saturated cyclic hydrocarbons, saturated polycyclic hydrocarbons, fluorenyl group, indan group, substituted indan group, and indan-1,3-dione.

As another example, the EphA2/4 inhibitor also can have the generic molecular structure of Formula II:

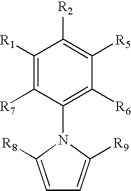

In Formula I₁, R₁ is R₃ or R₄; R₂ is R₃ or R₄, wherein R₁ and R₂ are not both R₃ or both R₄, wherein R₃ is —H, —OH, or —SH, wherein R₄ is —COOH, —CH₂—COOH, or —CH₂—CH₂—COOH; R₅, R₆, and R₇ are each independently —H or —OH; and R₈ and R₉ are each independently —CH₃, or —CH₂—CH₃, or —CH₂—CH₂—CH₃.

As another example, the EphA2/4 inhibitor can have the generic molecular structure of Formula III:

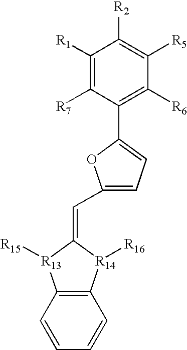

In Formula III, R₁ is R₃ or R₄; R₂ is R₃ or R₄, wherein R₁ and R₂ are not both R₃ or both R₄, wherein R₃ is —H, —OH, or —SH, wherein R₄ is —COOH, —CH₂—COOH, or —CH₂—CH₂—COOH; R₅, R₆, and R₇ are each independently —H or —OH; R₁₃ and R₁₄ are each C or S; R₁₅ and R₁₆ are each ═O or absent, wherein if R₁₃ is S, then R₁₅ is absent, wherein if R₁₃ is C, then R₁₅ is ═O, wherein if R₁₄ is S, then R₁₆ is absent, and wherein if R₁₄ is C, then R₁₆ is ═O.

The compounds of Formulas I and III can be further defined wherein if R₁₃ is S, then R₁₄ is C, and if R₁₄ is S, then R₁₃ is C or wherein R₁₃ and R₁₄ are each C and R₁₅ and R₁₆ are each ═O. The compounds of Formulas I, II and III also can be further defined wherein R₅ is —OH, R₆ is —H, and R₇ is —H; wherein R₅ is —H, R₆ is —H, and R₇ is —OH; or wherein R₅ is —H, R₆ is —H, and R₇ is —H. The compounds of Formulas I, II and III also can be further defined wherein R₈ is —CH₃ and R₉ is —CH₂—CH₃; wherein R₈ is —CH₂—CH₃ and R₉ is —CH₃; or wherein R₈ is —CH₃ and R₉ is —CH₃. The compounds of Formulas I, II and III also can be further defined wherein R₃ is —OH, and wherein R₄ is —COOH, —CH₂—COOH, or —CH₂—CH₂—COOH. The compounds of Formulas I, II and III also can be further defined wherein R₃ is —OH, and wherein R₄ is —COOH.

The disclosed EphA2/4 inhibitors can be used alone or in combination with one or more additional compounds or compositions. The disclosed compounds and compositions can comprise one or more EphA2/4 inhibitors. In some forms, the disclosed EphA2/4 inhibitors can be linked or coupled to one or more other compounds. For example, disclosed are compounds comprising an EphA2/4 inhibitor linked to a compound that binds to EphA4 noncompetitively relative to the EphA2/4 inhibitor. A compound that binds to EphA4 and/or EphA2 noncompetitively relative to an EphA4- and/or EphA2-specific inhibitor can be referred to as a co-binding compound. Particularly useful are co-binding compounds that bind EphA4 and/or EphA2 in the Eph ligand-binding channel (see Example 2). Compounds comprising linked Eph-specific inhibitor and co-binding compound can increase the binding affinity of the Eph-specific inhibitor. Disclosed herein are methods for identifying co-binding compounds by identifying compounds that bind EphA4 and/or EphA2 noncompetitively relative to an Eph-specific inhibitor.

Co-binding compounds can be linked to Eph-specific inhibitors in any suitable manner. In some forms, the co-binding compound can be directly coupled to the Eph-specific inhibitor. In some forms, the co-binding compound can be coupled via a linker to the Eph-specific inhibitor. The co-binding compound can be linked to any atom of the Eph-specific inhibitor provided that the Eph binding and specificity of the Eph-specific inhibitor is not significantly reduced. In some forms, the co-binding compound can be linked to the R₁₂ group of Formula I, the 5-membered ring of Formula II, or the bicyclic ring of Formula III.

The linker can have any suitable structure. In some forms, the linker can be —(CH₂)ₙ—R₁₇ wherein n is an integer from 1 to 10, —O—(CH₂)ₙ—R₁₇ where in n is an integer from 1 to 10, —(O—CH₂)ₙ—R₁₇ wherein n is an integer from 1 to 10, —(CH₂)ₙ—O—R₁₇ wherein n is an integer from 1 to 10, —(CH₂)ₙ—CH═R₁₈ wherein n is an integer from 1 to 10.

R₁₇ can be phenyl, biphenyl, naphtyl, tetrahydropyranyl (THP), trialkylsilyl wherein each alkyl chain has 1 to 3 carbons, dimethylsilyl, alicyclic cage group e.g. adamantly group or norbornyl group, alicyclic fused group e.g. naphtyl group, lactonyl group, saturated cyclic hydrocarbons, saturated polycyclic hydrocarbons, unsaturated cyclic hydrocarbons, unsaturated polycyclic hydrocarbons, pyrrolyl group, fluorenyl group, indan group, substituted indan group, indan-1,3-dione.

R₁₈ can be CH₂, saturated cyclic hydrocarbons, saturated polycyclic hydrocarbons, fluorenyl group, indan group, substituted indan group, and indan-1,3-dione.

The linker can be, for example, at its simplest, a bond between the EphA2/4 inhibitor and a compound (for example, a noncompetitive binder or co-binding compound). The linker can also be a linear, cyclic, or branched molecular skeleton having pendant groups covalently linking the EphA2/4 inhibitor to a compound.

Linking of the EphA2/4 inhibitor to a compound can be achieved by covalent means, involving bond formation with one or more functional groups located on the EphA2/4 inhibitor and a compound. Examples of chemically reactive functional groups that can be employed for this purpose include, for example, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of the EphA2/4 inhibitor and a compound can be effected using a linker that contains reactive moieties capable of reaction with such functional groups present in the EphA2/4 inhibitor and a compound. For example, an amine group of the EphA2/4 inhibitor can react with a carboxyl group of the linker, or an activated derivative thereof, resulting in the formation of an amide linking the two.

Examples of moieties capable of reaction with sulfhydryl groups include, for example, α-haloacetyl compounds of the type XCH₂CO— (where X═Br, Cl or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but can additionally be useful in coupling to amino groups under certain conditions. Reagents such as 2-iminothiolane (Traut et al., Biochemistry 12:3266 (1973)), which introduce a thiol group through conversion of an amino group, can be considered as sulfhydryl reagents if linking occurs through the formation of disulphide bridges.

Examples of reactive moieties capable of reaction with amino groups include, for example, alkylating and acylating agents. Representative alkylating agents include, for example:

(i) α-haloacetyl compounds, which show specificity towards amino groups in the absence of reactive thiol groups and are of the type XCH₂CO— (where X═Cl, Br or I), for example, as described by Wong Biochemistry 24:5337 (1979);

(ii) N-maleimide derivatives, which can react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds;

(iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which can react with amino, sulfhydryl, or phenolic hydroxyl groups;

(vii) chlorine-containing derivatives of s-triazines, which are very reactive towards nucleophiles such as amino, sufhydryl, and hydroxyl groups;

(viii) aziridines based on s-triazine compounds detailed above, for example, as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include, for example:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, for example, wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones can be reacted with amines to form Schiff's bases, which can be stabilized through reductive amination. Alkoxyl amino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

Functional groups in the EphA2/4 inhibitor and/or a compound can, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of the EphA2/4 inhibitor with a reactive chemical group of a compound without introducing additional linking material can be used.

Most commonly, however, the linker will include two or more reactive moieties, as described above, connected by a spacer element. The presence of such a spacer permits bifunctional linkers to react with specific functional groups within the EphA2/4 inhibitor and a compound, resulting in a covalent linkage between the two. The reactive moieties in a linker can be the same (homobifunctional linker) or different (heterobifunctional linker, or, where several dissimilar reactive moieties are present, heteromultifunctional linker), providing a diversity of potential reagents that may bring about covalent attachment between the EphA2/4 inhibitor and a compound.

Spacer elements in the linker can be, for example, linear or branched chains and can include, for example, a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In some forms, the linker can be described by Formula IV: G¹-(Z¹)ₒ—(Y¹)ᵤ—(Z²)ₛ—(R₂₀)—(Z³)ₜ—(Y²)ᵥ—(Z⁴)ₚ-G²

In Formula IV, G¹ is a bond between the EphA2/4 inhibitor and the linker; G² is a bond between the linker and a compound; Z¹, Z², Z³, and Z⁴ are each, independently, selected from O, S, and NR₁₉; R₁₉ can be hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₂₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ—(Y¹)ᵤ—(Z²)ₙ— to —(Z³)ₜ—(Y²)ᵥ—(Z⁴)ₚ-G². Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

### B. Detectable Agent

To aid in detection and quantitation of, for example, the location and binding of EphA2/4 inhibitors and other disclosed compounds and compositions, detectable agents can be incorporated into or coupled to EphA2/4 inhibitors and other disclosed compounds and compositions. As used herein, a detectable agent is any molecule that can be associated with a compound, directly or indirectly, and which results in a measurable, detectable signal, either directly or indirectly. Many such labels are known to those of skill in the art. Examples of detectable agents suitable for use in the disclosed methods and compositions are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as Quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin EBG, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the compound or composition with which they are associated is specifically bound to a target molecule, where such labels include, for example, “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Detectable agents that are incorporated into a compound or composition, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2-dioxetane-3-2′-(5′-chloro)tricyclo [3.3.1.1³′⁷]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detectable agents are also considered detectable agents. Any of the known detectable agents can be used with the disclosed EphA2/4 inhibitors and other disclosed compounds and compositions and methods to label and detect, for example, Eph binding, effects on Eph binding, location of binding in the disclosed methods. Methods for detecting and measuring signals generated by detectable agents are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detectable agent coupled to the antibody. As used herein, detection molecules are molecules which interact with a compound or composition to be detected and to which one or more detectable agents are coupled.

### C. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for identifying compounds that interact with EphA4, the kit comprising an EphA2/4 inhibitor composition and an EphA4 receptor. The kits also can contain an array of EphA4 receptors and an ephrin or other EphA4 ligand.

### D. Mixtures

Disclosed are mixtures formed by performing or preparing to perform the disclosed method. For example, disclosed are mixtures comprising an EphA2/4 inhibitor and EphA4 receptor.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### E. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising reagents for detecting EphA4 binding and an electronic instrument for detecting or analyzing EphA4 binding.

F. Data Structures and Computer Control

Disclosed are data structures used in, generated by, or generated from, the disclosed method. Data structures generally are any form of data, information, and/or objects collected, organized, stored, and/or embodied in a composition or medium. An Eph structure stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, use in assays to identify competitive and noncompetitive inhibitors of EphA4 and/or EphA2, use to treat cancer, use to treat cancer where EphA2 receptor activity is high in the cancer cells, and use to treat nerve damage or damaged nerves. Other uses include inhibition of tumor angiogenesis. Other uses are disclosed, apparent from the disclosure, and/or will be understood by those in the art.

### Method

Disclosed are methods relating to inhibitors of EphA4 and EphA2. For example, disclosed herein is a method of treating a subject, the method comprising administering to the subject an EphA2/4 inhibitor. The subject can be suffering or be at risk of suffering nerve injury. The subject can be suffering or be at risk of suffering cancer. The subject can have cancer cells in which EphA2 is activated above a threshold level. The method can further comprise measuring EphA2 receptor activity in cancer cells prior to administering the EphA2/4 inhibitor. The subject can be suffering or be at risk of suffering tumor angiogenesis.

Also disclosed herein is a method of identifying compounds, the method comprising determining the binding characteristics of a test compound in the presence and absence of an EphA2/4 inhibitor, wherein if the test compound exhibits noncompetitive binding with the EphA2/4 inhibitor, then the test compound is identified as a noncompetitive binder of EphA2 and/or EphA4 (relative to the EphA2/4 inhibitor). The binding characteristics can be, for example, to an Eph receptor, such as EphA4 receptor or EphA2 receptor. The method can further comprise linking the noncompetitive binder to an EphA2/4 inhibitor via a linker to form a linked EphA2/4 binder. The method can further comprise administering to a subject the linked EphA2/4 binder. The subject can have suffered or is at risk of suffering nerve injury. The subject can be suffering or is at risk from suffering cancer. The subject can have cancer cells in which EphA2 is activated above a threshold level. The method can further comprise measuring EphA2 receptor activity in cancer cells prior to administering the EphA2/4 inhibitor. The subject can be suffering or is at risk of suffering tumor angiogenesis.

Also disclosed herein is a method of identifying compounds that interact with EphA4, the method comprising bringing into contact a test compound, an EphA2/4 inhibitor composition, and an EphA4 receptor, wherein the EphA2/4 inhibitor composition comprises an EphA2/4 inhibitor; and detecting unbound EphA2/4 inhibitor composition, wherein a given amount of unbound EphA2/4 inhibitor composition indicates a compound that interacts with EphA4. The ability of the test compound to bind the EphA4 receptor and displace or prevent binding by the EphA2/4 inhibitor used in the method increases the amount of unbound EphA2/4 inhibitor. Thus, the increased amount of unbound EphA2/4 inhibitor indicates that the test compound interacts with the EphA4 receptor.

The EphA2/4 inhibitor composition can further comprise a moiety linked to the EphA2/4 inhibitor. The moiety linked to the EphA2/4 inhibitor can further comprise a detectable agent. The method can further comprise administering to a subject the test compound that interacts with EphA4. The subject in the disclosed method could have suffered or could be at risk of suffering nerve injury. The subject in the disclosed method can be suffering or is at risk of suffering from cancer. The subject in the disclosed method can have cancer cells in which EphA2 is activated above a threshold level. The method can further comprise measuring EphA2 receptor activity in cancer cells prior to administering the EphA2/4 inhibitor, and wherein in the subject can be suffering from or be at risk of suffering from tumor angiogenesis.

Also disclosed herein is a method of identifying a subject as having EphA4 receptor activity of interest, the method comprising measuring EphA4 receptor activity in the cell of a subject in the presence of an EphA2/4 inhibitor, wherein the subject has EphA4 receptor activity of interest if the measured EphA4 receptor activity differs from a reference EphA4 receptor activity by more than a threshold amount. The reference EphA4 receptor activity can be normal EphA4 receptor activity of a normal cell. The reference EphA4 receptor activity can be a non-pathological EphA4 receptor activity. The described method can be such that the measured EphA4 receptor activity is lower than the reference EphA4 receptor activity by more than the threshold amount.

Also disclosed herein is a method of identifying a subject as having EphA2 receptor activity of interest, the method comprising measuring EphA2 receptor activity in the cell of a subject in the presence of an EphA2/4 inhibitor, wherein the subject has EphA2 receptor activity of interest if the measured EphA2 receptor activity differs from a reference EphA2 receptor activity by more than a threshold amount. The reference EphA2 receptor activity can be normal EphA2 receptor activity of a normal cell. The reference EphA2 receptor activity can be a non-pathological EphA2 receptor activity. The described method can be such that the measured EphA2 receptor activity is lower than the reference EphA2 receptor activity by more than the threshold amount. The method for all the disclosed materials can further comprise administering to a subject to EphA2/4 inhibitor. The subject can have suffered or is at risk of suffering nerve injury. The subject can be suffering or is at risk from suffering cancer. The subject can have cancer cells in which EphA2 is activated above a threshold level. The method can further comprise measuring EphA2 receptor activity in cancer cells prior to administering the EphA2/4 inhibitor. The subject can be suffering or is at risk of suffering tumor angiogenesis. Also disclosed herein can be a pharmaceutical composition comprising an EphA2/4 inhibitor and a pharmaceutical acceptable carrier.

### A. Treating and Administration

In some forms, the disclosed methods involve treatment or subjects and/or administration of compounds. In particular, for example, subjects can be treated with the disclosed EphA2/4 inhibitors and compositions comprising EphA2/4 inhibitors; and EphA2/4 inhibitors and compositions comprising EphA2/4 inhibitors can be administered to a subject, cell or other recipient.

A cell can be in vitro. Alternatively, a cell can be in vivo and can be found in a subject. A “cell” can be a cell from any organism including, but not limited to, a bacterium.

The term “activity” as used herein refers to a measurable result of the interaction of molecules. Some exemplary methods of measuring these activities are provided herein.

The term “modulate” as used herein refers to the ability of a compound to change an activity in some measurable way as compared to an appropriate control. As a result of the presence of compounds in the assays, activities can increase or decrease as compared to controls in the absence of these compounds. An increase in activity can be, for example, at least 25%, at least 50%, and at least 100% compared to the level of activity in the absence of the compound. Similarly, a decrease in activity can be, for example, at least 25%, at least 50%, and at least 100% compared to the level of activity in the absence of the compound. A compound that increases a known activity is an “agonist”. One that decreases, or prevents, a known activity is an “antagonist.”

The term “inhibit” means to reduce or decrease in activity or expression. This can be a complete inhibition or activity or expression, or a partial inhibition. Inhibition can be compared to a control or to a standard level Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

By “treatment” is meant the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. A treatment that does not, in fact, produce any of the intended results is still considered a treatment as that term is used herein.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds. The term “suffering” from a disease or condition as used herein refers to a judgment made by a caregiver (e.g. physician, nurse, nurse practitioner, or individual in the case of humans; veterinarian in the case of animals, including non-human mammals) that a subject has or is suspected of having the disease or condition. These judgments can be made based on a variety of factors that are in the realm of a care giver's expertise, but that include the knowledge that the subject is ill, or will be ill, as the result of a condition.

The terms “at risk” of and “at risk of suffering” a disease or condition as used herein refers to a subject that may develop a disease or condition and/or symptoms of a disease or condition based on one or more criteria. Criteria can include, for example, test results, genetic background, ethnic group, diet, environmental exposure, exposure or risk of exposure to materials, compounds, environment, etc. that can cause or contribute to the development of the disease or condition, or a combination of such criteria. The at risk state can be judged by a caregiver (e.g. physician, nurse, nurse practitioner, or individual in the case of humans; veterinarian in the case of animals, including non-human mammals).

As used herein, a “subject” can be an individual. Thus, the “subject” can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

The terms “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

In some forms, the compounds described herein can be administered to a subject comprising a human or an animal including, but not limited to, a mouse, dog, cat, horse, bovine or ovine and the like, that is in need of alleviation or amelioration from a recognized medical condition.

By “pharmaceutically acceptable” is meant a material that is not biologically, clinically or otherwise undesirable, i.e., the material can be administered to an individual along with the relevant active compound without causing clinically unacceptable biological effects or interacting in a deleterious manner with any of the other components of the pharmaceutical composition in which it is contained.

Any of the disclosed compounds can be used therapeutically in combination with a pharmaceutically acceptable carrier. The compounds described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

The pharmaceutical compositions described herein can include, but are not limited to, carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The compounds and pharmaceutical compositions described herein can be administered to the subject in a number of ways depending on whether local or systemic treatment is desired, and on the area to be treated. Thus, for example, a compound or pharmaceutical composition described herein can be administered as an ophthalmic solution and/or ointment to the surface of the eye. Moreover, a compound or pharmaceutical composition can be administered to a subject vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration can include powders or granules, suspensions or solutions in water or non-aqueous media, capsules, sachets, or tablets. Thickeners, flavorings, diluents, emulsifiers, dispersing aids or binders can be desirable.

By the term “effective amount” of a compound as provided herein is meant a nontoxic but sufficient amount of the compound to provide the desired result. The exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds described herein are large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject in need Eph-specific inhibitor for the treatment of nerve damage, spinal cord injury, brain damage, cancer, angiogenesis, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

Further, subjects for administration of the disclosed compounds and compositions can be identified by assessing EphA4 and/or EphA2 expression and/or activity in the subject and/or in relevant tissues and/or cells of the subject.

### B. Binding

The disclosed methods can involve determining binding characteristics of EphA2/4 inhibitors and other disclosed compounds and compositions, such as test compounds. As used herein, “binding characteristics” refers to any one or combination of features of binding, including, for example, association constants, dissociation constants, on rates of binding, off rates of binding, changes in binding in the presence or absence of other compounds, and relative binding constants of different compounds. Binding characteristics can consist of one such feature or any combination of such features. Suitable binding characteristics can readily be chosen by those of skill in the art. For example, where compounds that exhibit noncompetitive binding are identified, those of skill in the art can choose and determine binding characteristics that relate to or are indicative of noncompetitive binding. Examples of useful binding characteristics include association constants, dissociation constants, on rates of binding, off rates of binding, changes in binding in the presence or absence of other compounds, and relative binding constants of different compounds. Many other characteristics of binding interactions are known and can be used.

Noncompetitive binding is analogous to partially competitive inhibition in enzyme kinetics and generally can be analyzed using similar equations and graphs. In noncompetitive binding, the binding of a first ligand does not affect the binding of a second ligand. Noncompetitive and competitive binding can give similar graphs for some types of binding analysis graphs. One way to identify noncompetitive binding is to plot the slope of the straight lines of a double reciprocal plot against the concentration of the second ligand. If the plot is not a straight line, it indicates noncompetitive binding. Competitive binding would give a straight line in such a plot.

1. Reference Level and Threshold Level

A reference level of Eph activity refers to any level of Eph activity that can be relevant for comparing with a measured Eph activity. For example, the normal EphA4 or EphA2 receptor activity in a normal cell can be sued as a reference level to determine if an abnormal EphA4 or EphA2 receptor activity is present in, for example, a cell, tissue, or sample. As described herein, higher than normal activity of EphA2 (in, for example, cancer cells or tumor tissue) can indicate that inhibition of that abnormal activity is appropriate. As another example, EphA4 may normally have a low or undetectable activity but a higher or detectable activity in damaged nerve cells and/or nerve tissue. Detection of EphA4 receptor activity can indicate inhibition of that abnormal activity is appropriate. In such cases, the reference level of EphA4 can be considered the low or undetectable activity, for example. Because Eph activity can be associated with pathological effects, such as, for example, nerve cell and nerve tissue damage, cancer, and tumor-related angiogenesis, Eph activity that is below the level of activity associated with such pathological conditions and effects can be considered non-pathological level of Eph activity, such as a non-pathological level of EphA4 receptor activity or of EphA2 receptor activity. A non-pathological Eph activity can be useful as a reference level.

A threshold level refers to the difference from a reference level of activity beyond which a measured level of activity can be considered meaningfully or actionably different from the reference level. The threshold level can be a small increment or a larger increment depending on the purpose of the measurement and the significance to be assigned to a measurement that differs by the threshold level.

For detection of EphA2/4 inhibitors and other disclosed compounds and compositions, and for detection of binding of such, detectable agents can be used and detected. The method of detection depends on the type of label used. Those of skill in the art know how to measure various detectable agents and such methods can be used with the disclosed methods. Many such methods and labels are know and can be used in the disclosed methods.

### EXAMPLES

### C. Example 1

### Analysis of Small molecule Inhibitors of Ephrin Binding to EphA4 and Eph2B Receptors

The method described herein utilizes a high throughput screening approach to identify small molecular weight compounds that can inhibit ligand binding to the EphA4 receptor and Eph2B receptor. The screen identified two exemplary compounds, isomeric 2,5-dimethylpyrrolyl benzoic acid derivatives, that selectively inhibit ephrin binding to EphA4 and EphA2 as well as the functions of these receptors in live cells.

1. Materials and Methods

Chemical Library Screening for EphA4 Inhibitors—A 96-well format in vitro assay was used for compound screening. Polystyrene high binding capacity plates (Corning, Corning, N.Y.) were incubated overnight at 4° C. with 2 μg/mL streptavidin (Pierce Biotecnology, Rockford, Ill.) diluted in borate buffer 0.1 M pH 8.7 and then coated by overnight incubation with 0.1 μM of biotinylated KYL peptide (Murai et al. (2003) Mol Cell Neurosci 24(4), 1000-1011) in binding buffer (Tris-buffered saline (TBS; 150 mM NaCl, 50 mM Tris-HCl, pH 7.5) with 1 mM CaCl₂ and 0.01% Tween 20). Compounds were added to the wells at 10 μg/mL in 100% dimethylsulfoxide (DMSO) together with EphA4 alkaline phosphatase fusion protein (EphA4 AP) produced from transfected cells. Cell culture medium containing the secreted EphA4 AP was diluted 1:16 in binding buffer. The mixture was incubated for 3 hours at room temperature before washing with binding buffer and adding p-nitrophenylphosphate (pNPP) (Pierce Biotecnology, Rockford, Ill.) as the substrate. After 1 hour the reaction was stopped by adding 2N NaOH and the absorbance at 405 nm was measured using an ELISA plate reader Alkaline phosphatase activity from wells where alkaline phosphatase (AP) was added instead of EphA4 AP was subtracted as background. The inhibitory activity of the compounds was calculated by dividing the absorbance observed in the presence of each compound and the absorbance from wells where no compound was added. Compounds with inhibitory activity higher than 50% were considered hits. The inhibitory activity of the hits was confirmed by repeating the assay, hence multiple experiments.

ELISA Assays and K, Determination—Protein A-coated wells (Pierce Biotecnology, Rockford, Ill.) were used to immobilize ephrin Fc fusion proteins (R&D Systems, Minneapolis, Minn.). Compounds at different concentrations were incubated with EphA4 AP (Cheng and Flanagan, (1994) Cell 79(1), 157-168) or EphA2 AP (Koolpe et al. (2002) J Biol Chem 277(49), 46974-46979) for 3 hours. Alternatively, Eph receptor Fc fusion proteins were immobilized on protein A-coated wells and ephrin-A5 AP (Menzel et al. (2001) Developmental Biology 230(1), 74-88) or ephrin-B2 AP (GeneHunter, Nashville, Tenn.) were added with the compounds. The amount of bound AP-fusion protein was quantified using pNPP as the substrate. Alkaline phosphatase activity from wells with Fc only was subtracted as background. To confirm that the binding of the compounds to EphA4 was reversible, the compounds were removed and the wells were incubated in binding buffer for 3 hours before washing and incubating with ephrin AP fusion proteins. Under these conditions, no inhibition of ephrin binding was observed indicating the reversible binding of the compounds. Further control experiments verified that the compounds do not inhibit the activity of alkaline phosphatase in solution and also do not inhibit binding of EphA4 AP to an anti-EphA4 antibody (R&D Systems, Minneapolis, Minn.) immobilized to protein G-coated plates (Pierce Biotechnology, Rockford, Ill.), ruling out non specific inhibitory effects.

To calculate the inhibition constant (KO values, the binding of ephrin-A5 AP to EphA4 Fc immobilized on protein A-coated wells was measured in the absence and in the presence of the compounds at different concentrations. Each data set was fitted to the Michaelis-Menten equation: B=Bₘₐₓ [S]/(K_{D}+[S]), where [S] is the concentration of ephrin AP fusion protein and K_{D} is the dissociation constant observed in the absence or in the presence of the compound, using non linear regression and the program GraphPad (Prism). To evaluate whether the inhibition is competitive, noncompetitive or uncompetitive the K_{D} and Bₘₐₓ values were determined at different compound concentrations. The Kᵢ was obtained from the linear regression plot of K_{D}/Bₘₐₓ as a function of the concentration of the inhibitor according to: K_{D}/Bₘₐₓ=(K_{D}[S])/(Kᵢ·Bₘₐₓ)+K_{D}/Bₘₐₓ. Alternatively, Kᵢ values were obtained from the dose response curves, using the Cheng-Prusoff equation: Kᵢ=IC₅₀/(1+[S]/K_{D}) (XX38, Cheng, Y., and Prusoff, W. H. (1973) Biochemical pharmacology 22(23), 3099-3108). Ephrin-A5 AP concentration were calculated from alkaline phosphatase activity (Flanagan et al. (2000) Methods in Enzymology. 327, 19-35).

Chemical synthesis—Compounds were purchased from ChemBridge; with the exception of compound 29 (Matrix Scientific, Columbia, S.C.), compounds 14 and 33 (Sigma-Aldrich, St. Louis, Mo.), compound 21 (Key Organics, Cornwall, UK), compounds 8 and 39 (ChemDiv, San Diego, Calif.) and compounds 3, 4, 5, 7, 19, 22, 26, 27, 37, 40, 41, 42, 47, 54 and 55, which were synthesized as described elsewhere herein. Furthermore, as a control compound 1 was also synthesized as well as purchased from Interbioscreen (Moscow, Russia).

For the synthesis of compounds 1, 26, 27, 37, 39, 41, 42, and 54, a 15 mL glass pressure vessel was charged with the appropriate aniline (1.0 mmol), 2,5-hexanedione (1.2 mmol), p-toluenesulfonic acid (0.2 mmol), and toluene (5.0 mL). The mixture was stirred and refluxed for 24 hours. After evaporation of the toluene, the crude product was purified first by flash chromatography (ethyl acetate:hexanes) and then by reverse phase chromatography. The final products were lyophilized to give solids in yields ranging from 47% to 82%. Final product purities of greater than 95% were confirmed by ¹H NMR or liquid chromatography/mass spectrometry.

For the synthesis of compounds 3, 4, 7, and 19, a 35 mL microwave tube was charged with the appropriate aniline (1.0 mmol), 2,5-hexanedione (1 2 mmol), p-toluenesulfonic acid (0.2 mmol), and ethanol (5.0 mL). The mixture was heated under microwave irradiation at 180° C., for 5 minutes. The solvent was then evaporated and the residue was subjected to flash chromatography (0-15% ethyl acetate/hexanes or 0-10% methanol/dichloromethane) and then reverse phase chromatography. The final products were lyophilized to give products in yields ranging from 30% to 80%. Final product purities of greater than 95% for compounds 3 and 19 and greater than 80% for compound 4 were confirmed by ¹H NMR and/or liquid chromatography/mass spectrometry.

For the synthesis of compounds 5, 22, 40, and 47, the appropriate aryl halide (0.5 mmol) was mixed with 2,5-dimethylpyrrole (0.7 mmol), CuI (0 1 mmol), N-methylglycine (0.2 mmol), and potassium carbonate (1 5 mmol) in dimethylformamide (5.0 mL). The mixture was placed in a sealed glass vial and irradiated under microwave conditions at 200° C. for 20 minutes. The resulting mixture was cooled, filtered and concentrated in vacuo. The resulting residue was dissolved in acetonitrile and purified via reverse phase chromatography. After lyophilization, the product pyrroles were furnished as solids with yields ranging from 26% to 57%. Final product purities of greater than 95% were confirmed by ¹H NMR or liquid chromatography/mass spectrometry. The identity and purity of all the synthesized compounds and compound 1 purchased from Interbioscreen was verified by liquid chromatography/mass spectrometry.

Measurement of Receptor Tyrosine Phosphorylation in Cells—HT22 neuronal cells, which endogenously express EphA4, are derived from immortalized mouse hippocampal neurons (Li et al. (1997) Neuron 19(2), 453-463). COST cells, which endogenously express EphA2, EphB2 and the EGF receptor (EGFR), were obtained from ATCC. Both cell lines were grown in Dulbecco's Modified Eagle's Medium (DMEM) (Mediatech, Inc, Herndon, Va.) with 10% fetal bovine serum (FBS) (Hyclone, Logan, Utah) and Pen/Strep. For EphA4 immunoprecipitations, HT22 cells were serum-starved overnight in 0.5% FBS in DMEM and incubated for 15 min with the compounds or DMSO as a control. The cells were then stimulated with 0.5 μg/mL ephrin-A5 Fc, ephrin-A4 Fc or Fc for 20 min in the continued presence of the compounds. After stimulation the cells were lysed in modified RIPA buffer (1% Triton X-100, 1% Na deoxycholate; 0.1% SDS; 20 mM Tris; 150 mM NaCl; 1 mM EDTA) containing 10 μM NaF, 1 μM sodium pervanadate and protease inhibitors. The protein concentration was calculated using the BCA protein assay kit (Pierce Biotecnology, Rockford, Ill.). Cell lysates were immunoprecipitated with 4 μg anti-EphA4 antibody (Soans et al. (1994) Oncogene 9(11), 3353-3361). For EphA2 and EphB2 immunoprecipitations, serum-starved COS7 cells were stimulated with 0.1 μg/mL ephrin-A1 Fc or 0.5 μg/mL ephrin-B2 Fc, respectively. The cells were then lysed and incubated with 2 μg of anti-EphA2 antibody (Millipore-Upstate, Inc, Temecula, Calif.) or 7 μg anti-EphB2 antibody made to a GST fusion protein of the EphB2 carboxy-terminal tail (Holash and Pasquale, (1995) Developmental Biology (Orlando) 172(2), 683-693). To assess EGFR phosphorylation, COS7 cells were serum-starved overnight in 0.2% FBS in DMEM. The cells were preincubated with the compounds as described elsewhere herein and then stimulated for 15 mM with 0.1 μM EGF. To assess inhibition of EphA2 phosphorylation in response to endothelial cell stimulation with tumor necrosis factor alpha (TNFα), HUVEC cells obtained from Cascade Biologics (Portland, Oreg.) were grown in Medium 200 supplemented with low serum growth supplements (Cascade Biologics), 10% FBS, Pen/Strep and fungizone. The cells were serum starved overnight in 2% FBS containing medium before adding 7 nM TNFα together with the compound or DMSO for 2 hours. Immunoprecipitates and lysates were probed by immunoblotting with anti-phosphotyrosine antibody (Millipore, Inc, Temecula, Calif.) and reprobed with antibodies to the respective Eph receptors or anti-EGF receptor antibodies (Santa Cruz Biotechnology, Santa Cruz, Calif.) followed by a secondary anti-IgG peroxidase-conjugated antibody (GE Healthcare, UK). The EphA2 and EphA4 antibodies used for immunoblotting were from Invitrogen/Zymed Laboratories (San Francisco, Calif.).

MTT Assay—The cytotoxicity of the compounds was measured using the 3-(4,5-dimethylthiazol-2-yl)-2,5-diphenyltetrazolium bromide (MTT) colorimetric assay. Cells were seeded in 96-well plates and treated with compounds or DMSO starting 3, 2 or 1 day before they reached 100% confluency. For the assay, MTT (Sigma-Aldrich, St. Louis, Mo.) was added at a final concentration of 0.5 μg/mL and incubated with the cells for 3 hours. The resulting formazan crystals were then solubilized by addition of 100% DMSO. The absorbance in each well was measured at 570 nm using an ELISA plate reader. The results were expressed as the ratio of the absorbance of the cells treated with the compounds and left untreated.

Growth Cone Collapse Assay—Nasal retina explants from embryonic day 6 chicken embryos were cultured on coverslips coated with 200 μg/mL poly-L-lysine and 20 μg/mL lamining for 12 to 24 hours in DMEM/F-12 culture medium containing 10% FBS and 0.4% methylcellulose. Three hours before adding the Fc fusion proteins, the medium was changed to DMEM/F-12 without methylcellulose. The explants were incubated for 15 min with the KYL peptide or the chemical compounds and then stimulated for 30 min with 1 μg/mL preclustered ephrin-A5 Fc or Fc as a control. The cultures were fixed for 30 min in 4% paraformaldehyde/4% sucrose in phosphate buffered saline (PBS), permeabilized in 0.1% Triton X-100 in PBS and stained with rhodamine-conjugated phalloidin (Invitrogen, Carlsbad, Calif.). Cells were photographed under a fluorescence microscope and growth cones where scored in a blinded manner as collapsed when no lamellipodia or filopodia were present at the tip of the neurite.

PC3 Cell Retraction Assay—PC3 cells were plated on glass coverslips and grown in RPMI 1640 medium (Mediatech, Inc, Herndon, Va.) with 10% FBS and Pen/Strep. After 17 hours the cells were starved for 3 hours in 0.5% FBS in DMEM and then incubated for 40 min with the compounds or DMSO, before stimulation for 10 min with 0.5 μg/mL of ephrin-A1 Fc or Fc as a control. The cells were fixed in 4% formaldehyde in PBS, permeabilized in 0.5% Triton X-100 in TBS and stained with rhodamine-conjugated phalloidin (Invitrogen, Carlsbad, Calif.) and DAPI. Cells were photographed under a fluorescence microscope and cell area was measured in a blinded manner using ImageJ software. Cells having rounded shape and area equal or below 20% of the area of Fc control-treated cells were considered as retracting.

2. Results

Chemical Library Screening to Identify Compounds That Inhibit Ligand Binding to the EphA4 Receptor—To identify small molecule inhibitors of ligand binding to the EphA4 receptor, an assay was designed that takes advantage of a peptide ligand previously identified by phage display (Murai et al. (2003) Mol Cell Neurosci 24(4), 1000-1011). The peptide—designated KYL—has some sequence similarity with the ephrin-A G-H loop, which mediates high-affinity binding to Eph receptors (Himanen et al. (2001) Nature 414(6866), 933-938). Furthermore, the KYL peptide was shown to competitively inhibit ephrin binding to EphA4, indicating that it targets the high-affinity ligand-binding site of the receptor (Murai et al. (2003) Mol Cell Neurosci 24(4), 1000-1011).

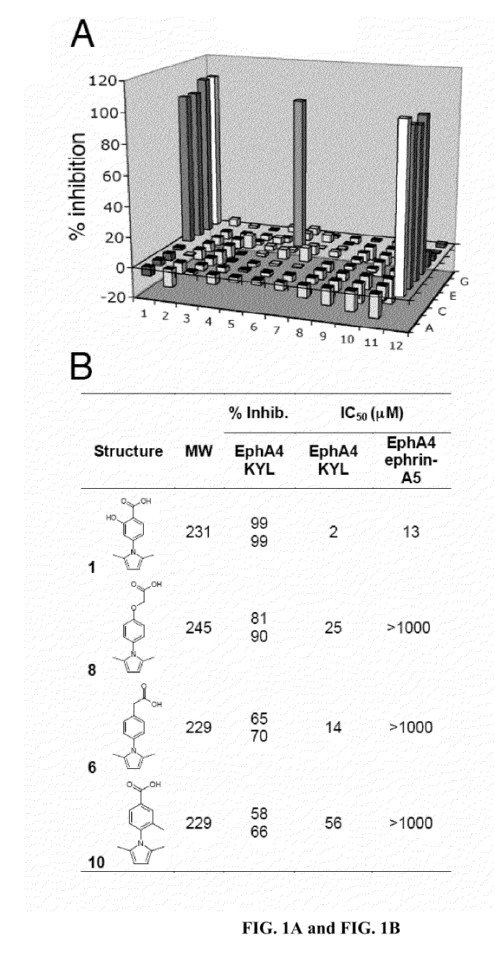

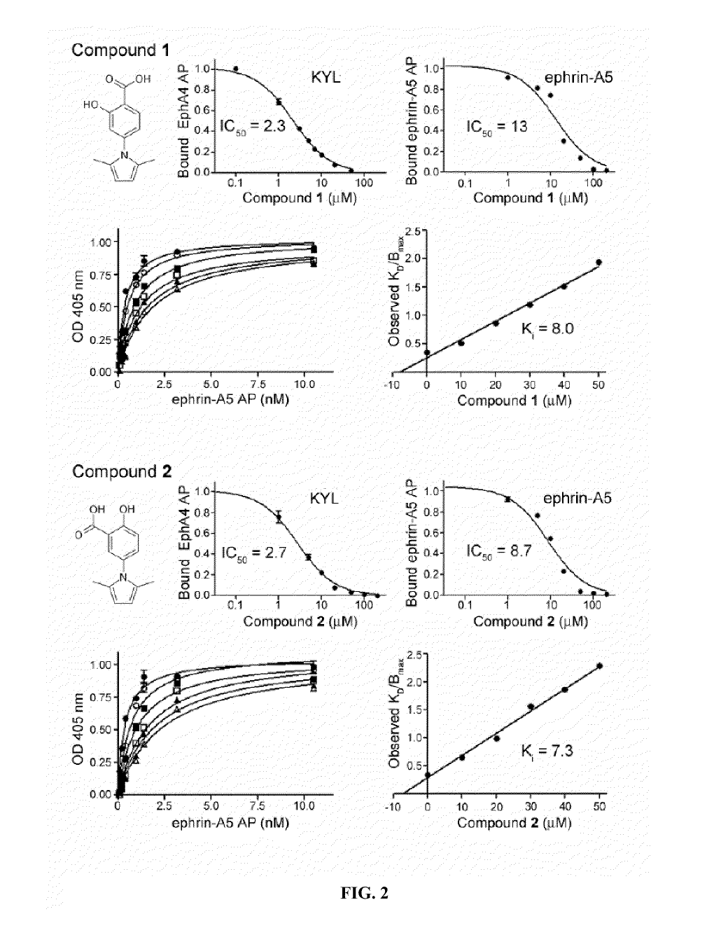

The biotinylated KYL peptide was immobilized on streptavidin-coated ELISA wells and binding of the extracellular domain of EphA4 fused to alkaline phosphatase (EphA4 AP) was measured in the presence of chemical compounds. 10,000 compounds were screened from the DIVERSet™ library (ChemBridge, Inc.) at 10 μg/mL in a 96-well format, which identified 43 compounds that reproducibly inhibited EphA4 AP binding by more than 50% in both the original screen and in a rescreen of the hits (FIG. 1A). Four of the compounds shared a 2,5-dimethylpyrrolylbenzene scaffold and inhibited EphA4 AP binding to the KYL peptide with IC₅₀ values ranging from 3 to 56 μM (FIG. 1B). Importantly, compound 1,2-hydroxy-4-(2,5-dimethyl-1-pyrrolyl)benzoic acid, also inhibited binding of ephrin-A5 AP to the EphA4 extracellular domain with an IC₅₀ value of 13 μM (FIG. 2). Control experiments also verified that the compound binds reversibly to EphA4 and does not inhibit alkaline phosphatase activity or protein-protein interactions other than EphA4 ligand binding. Thus, compound 1 can effectively inhibit binding of the EphA4 receptor to both a synthetic peptide ligand and a natural ephrin ligand.

Two 2,5-Dimethylpyrrolyl Benzoic Acid Derivatives Selectively Target the EphA4 and EphA2 Receptors—49 additional compounds belonging to the same class as compound 1 were obtained from ChemBridge and other sources were examined in ELISA experiments for their ability to inhibit EphA4-KYL and EphA4-ephrin-A5 binding. Compound 2—a 1,2-isomer of compound 1—also inhibited binding of ephrin-A5 AP to immobilized EphA4 (FIG. 2). The IC₅₀ value for inhibition of EphA4-KYL peptide binding by compound 2 was 3 μM and for inhibition of EphA4-ephrin-A5 binding was 9 μM (FIG. 2). By measuring ephrin-A5 AP binding curves at different compound concentrations, it was determined that compounds 1 and 2 competitively inhibit EphA4-ephrin-A5 binding with Kᵢ values of 8 μM and 7 μM, respectively (FIG. 2). These data indicate that compounds 1 and 2 target the high affinity ephrin-binding pocket of the Eph receptors. The Kᵢ value can also be obtained from the IC₅₀ value and the dissociation constant (K_{D}) for receptor-ligand binding, using the Cheng-Prusoff equation. The Kᵢ values for compounds 1 and 2 calculated from the inhibition curves shown in FIG. 2 were 10 and 6 μM, respectively. Ki values calculated from other inhibition curves obtained using different ephrin concentrations ranged from 6 to 10 μM for compound 1 and from 6 to 8 μM for compound 2.

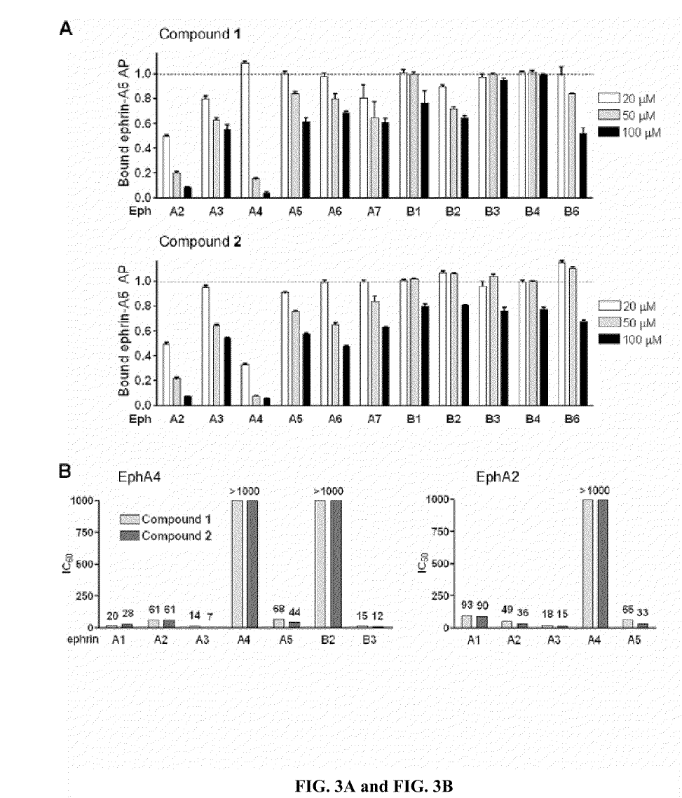

Despite the small size of the compounds and the ability of each ephrin ligand to bind promiscuously to different Eph receptors, compounds 1 and 2 efficiently inhibited ephrin binding only to EphA4 and EphA2 but not any of the other EphA or EphB receptors examined (FIG. 3A). Assuming that compound 1 and 2 also competitively inhibit ligand binding to the EphA2 receptor, the Cheng-Prusoff equation was used to calculate the Kᵢ values for inhibition of EphA2-ephrin-A5 binding, which ranged from 11 to 14 μM for compound 1 and from 10 to 13 μM for compound 2. Both compounds inhibited binding of most ephrin ligands to EphA4, except for ephrin-A4 and ephrin-B2, indicating differences in how these ephrins bind to EphA4. Similar selectivity was obtained for EphA2-ephrin-A binding (FIG. 3B), indicating that ephrin-A4 also interacts with EphA2 differently than other ephrins.

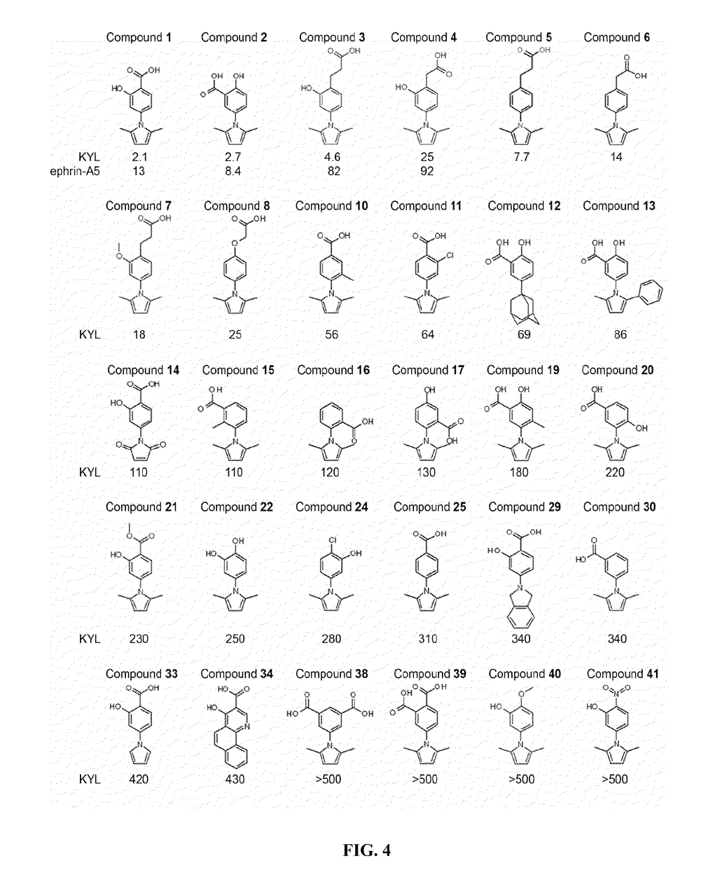

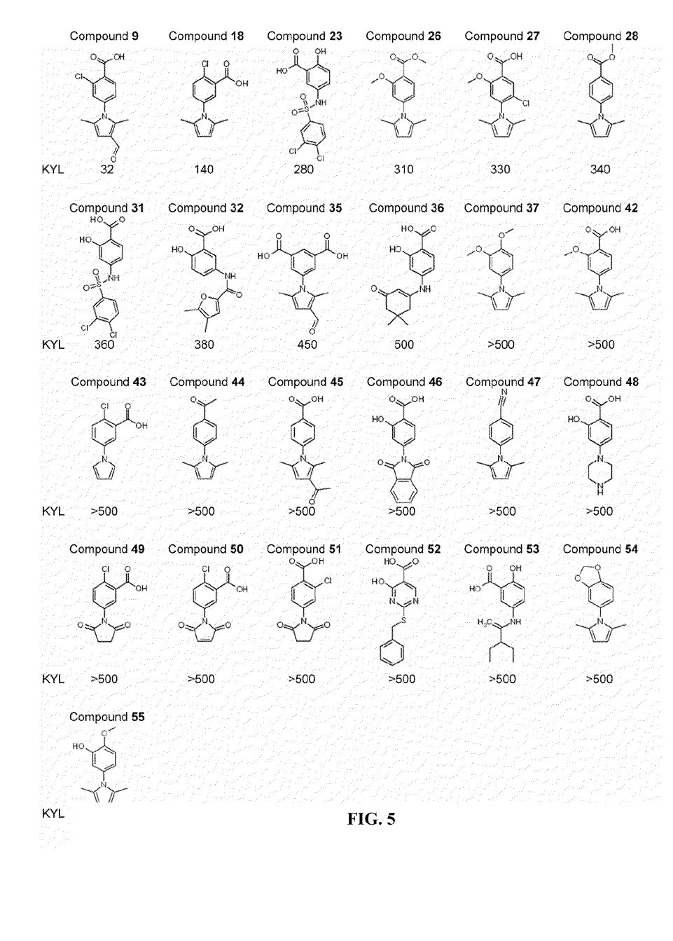

Structure-Activity Relationship Analysis of Small Molecules with a 2,5-Dimethylpyrrolyl Benzene Scaffold and Related Compounds—IC₅₀ values for compounds structurally related to compound 1 and 2 were measured to provide information that can explain structural features to improve the potency of compounds related to 1 and 2. The compounds were either available from commercial sources or synthesized accordingly (FIGS. 4 and 5). Among the 49 analogs (compounds 5, 6, 8-18, 20-55), none detectably inhibited EphA4-ephrin-A5 binding. Even small changes to the structures of compounds 1 and 2 abolished the ability to inhibit ephrin binding. For example, compounds lacking the hydroxyl group on the benzene ring (compounds 25, 30), compounds having a carboxylic group in place of the hydroxyl group (compound 38, 39), compounds having a hydroxyl group in place of the carboxylic group (compound 22), and compounds with the carboxylic group and the hydroxyl group placed at different positions on the benzene ring(compounds 17, 20) did not show measurable inhibition of EphA4-ephrin binding. These results indicate that the presence of the hydroxyl and carboxylic moieties and their position on the benzene ring are crucial for the activity of the compounds. The loss of activity was confirmed by the loss of inhibitory activity when the hydroxyl or carboxylic group was substituted with a nitro group (compounds 41) or a chlorine atom (compounds 11, 24). In addition, no inhibition of EphA4-ephrin binding was observed with the methyl-ester derivative of compound 1 (compound 21) or when a methoxy group replaced the carboxylic group of compound 1 (compound 40), indicating that the carboxylic group can be involved in hydrogen bonding with EphA4.

Alternatives substitutions to the 2,5-dimethylpyrrolyl groups were also tested (FIG. 4). Structurally modifying or eliminating the two methyl groups on the pyrrole ring (compounds 14, 33) abolished the inhibitory activity with the ephrin and almost abolished the inhibitory activity with the KYL peptide in the case of compound 33. This outcome can be a result of either modulation of the dihedral angle of the benzene and pyrrole rings or of favorable lipophilic interactions of the pyrrole methyl groups with the binding site in EphA4. Another explanation to the behavior of compound 33, which is inactive despite being very similar to the two most potent compounds, is that the absence of the methyl groups makes the pyrrole ring more unstable. However, this explanation could be less likely because compounds where the pyrrole ring is substituted with an adamantyl or isoindolyl group (compounds 12, 29) or by two phenyl groups fused with the hydroxyl benzoic acid ring (compound 34) would be expected to be more stable than compounds with the pyrrole ring, but still do not inhibit EphA4 ligand binding. Inserting a bigger moiety, like a benzene ring in place of one of the methyl groups (compound 13) also abolished activity which could be due by creating increased steric hindrance.

Although none of the compounds tested detectably inhibited ephrin binding to EphA4, their IC₅₀ values for inhibition of EphA4-KYL peptide binding were used as a guide to design modified versions of compounds 1 and 2 that might have increased potency (FIG. 4). For example, compounds 5, 6, and 8, which have a phenoxy acetic acid, a phenyl acetic acid and a phenyl propanoic acid in place of the benzoic acid in compound 25, inhibited EphA4-KYL binding with 10 to 40 fold lower IC₅₀ values than compound 25. This indicated that substituting the carboxylic group of compound 1 with these other groups can improve its inhibitory activity. Compounds 3 and 4 were therefore synthesized. However, these compounds inhibited EphA4-KYL and EphA4-ephrin-A5 interactions with lower potency than compound 1. Nevertheless, compounds 3 and 4 are still selective EphA4 and EphA2 inhibitors and show the same differential inhibition of ephrin binding as compounds 1 and 2. Compound 7, an intermediate in the synthesis of compound 4, was also tested and found not to inhibit ephrin binding to EphA4.

The IC₅₀ values for compounds 10 and 15 were approximately 6 and 3 fold lower than those for compounds 25 and 30, which only differ for the absence of a methyl group attached to the benzene ring. This indicated that adding a methyl group to the benzene ring of compounds 1 and 2 can improve inhibitory activity. Compound 19 was synthesized corresponding to compound 2 with an added methyl group at the same position as in compound 10. However, compound 19 did not inhibit EphA4-ephrin-A5 binding and inhibited EphA4-KYL binding only when present at high concentration.

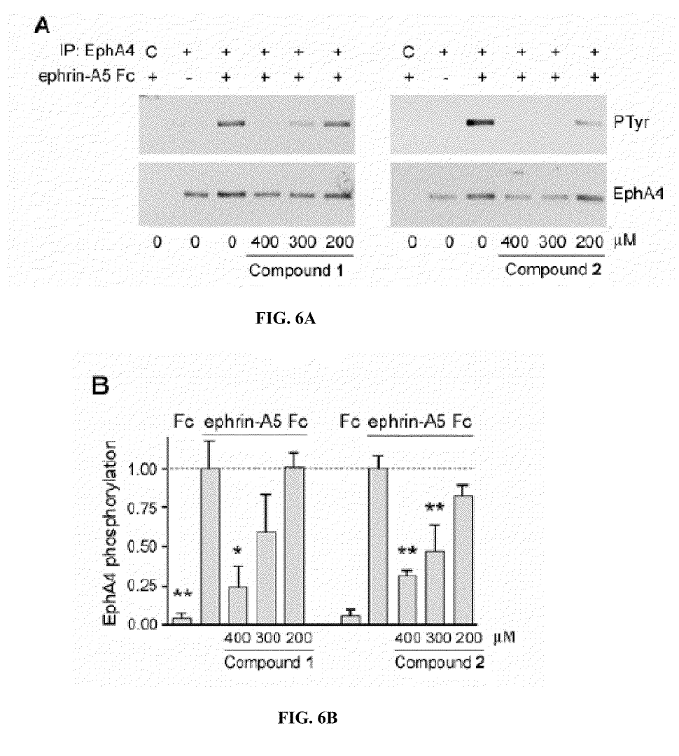

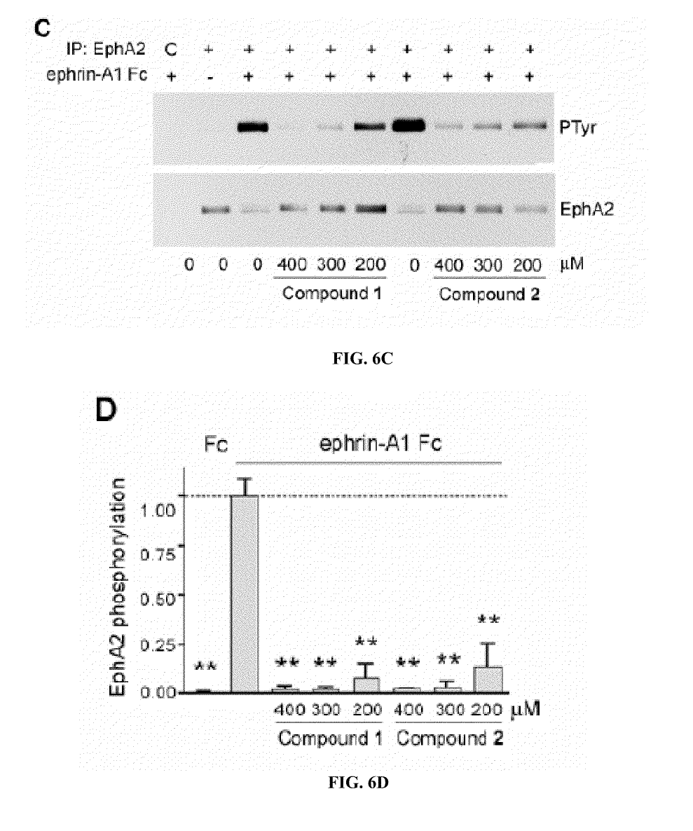

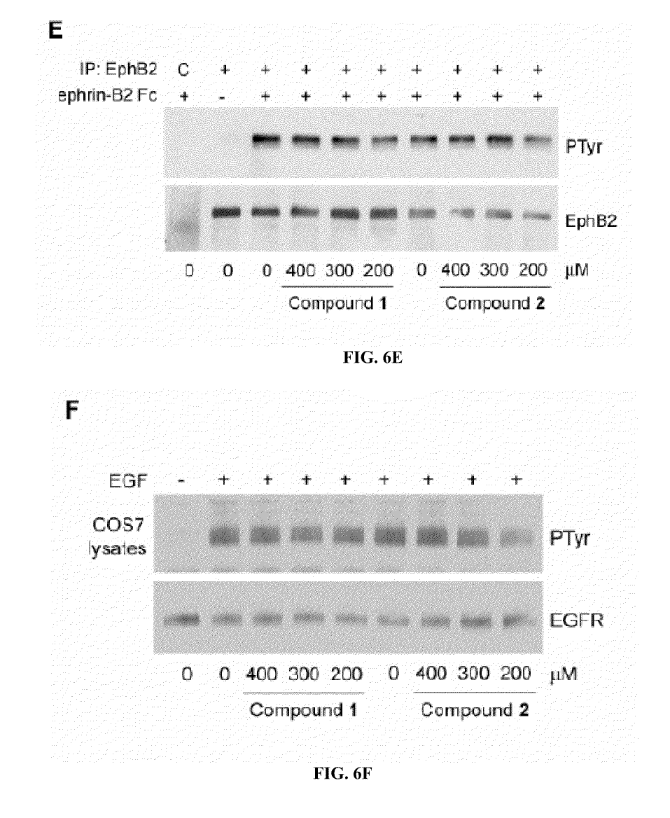

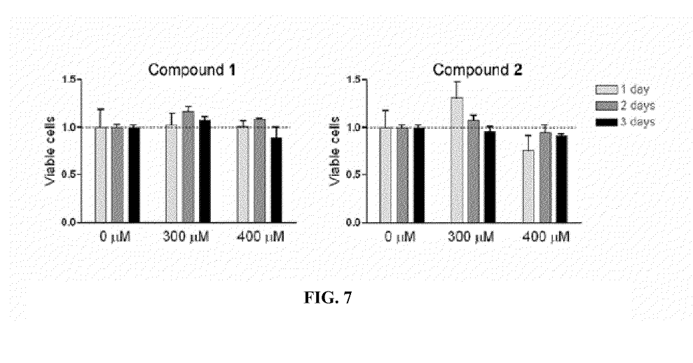

Compounds 1 and 2 Selectively Inhibit EphA4 and EphA2 Activation by Ephrin in Cells without Showing Toxicity—Compounds 1 and 2 were the best antagonists in the ELISA assays. Therefore, the ability of these two compounds to inhibit ephrin-induced EphA4 and EphA2 tyrosine phosphorylation (indicative of receptor activation) was examined in cultured cells. Both compounds blocked tyrosine phosphorylation of endogenous EphA4 in HT22 neuronal cells stimulated with ephrin-A5 Fc, although the concentrations needed where higher than those effective in the ELISA assays (FIGS. 6A and 6B). Both compounds also inhibited tyrosine phosphorylation of endogenous EphA2 in COS7 cells stimulated with ephrin-A1 Fc (FIGS. 6C and 6D) and in HUVE endothelial cells treated with TNFα to stimulate expression of endogenous ephrin-A1 (Pandey et al. (1995) Science 268(5210), 567-569). Furthermore, the compounds prevented ephrin-dependent degradation of EphA2 (Walker-Daniels et al. (2002) Mol Cancer Res 1(1), 79-87), as expected from inhibition of ephrin binding. Consistent with the selectivity observed in the ELISA assays, compounds 1 and 2 did not inhibit EphA4 phosphorylation in cells stimulated with ephrin-A4 or phosphorylation of endogenous EphB2 in COS7 cells stimulated with ephrin-B2 Fc (FIG. 6E). Moreover, the compounds did not inhibit phosphorylation of the epidermal growth factor (EGF) receptor in COS7 cells stimulated with EGF (FIG. 6F) or overall tyrosine phosphorylation in COS and HT22 cells. Assessment of cell viability using the MTT assay did not reveal any toxicity of compounds 1 and 2 at concentrations up to 400 μM for several days (FIG. 7).

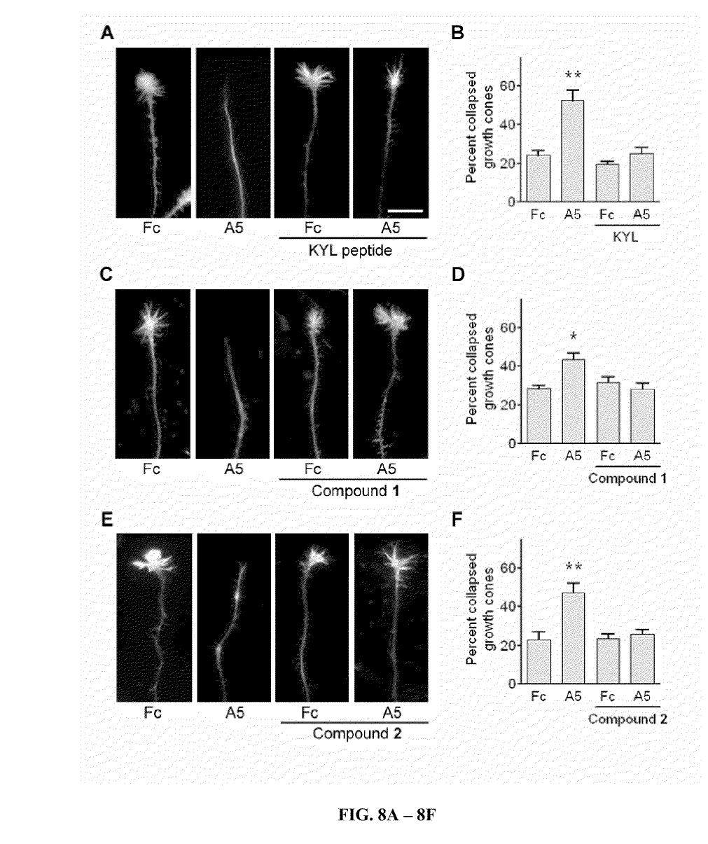

Compounds 1 and 2 Inhibit EphA4-Dependent Growth Cone Collapse in Retinal Neurons—Growth cones are enlarged structures at the leading edge of axons, and control the growth of the axons towards their synaptic targets by responding to environmental cues (Dickson, (2002) Science 298(5600), 1959-1964, Wen and Zheng, (2006) Curr Opin Neurobiol 16(1), 52-58). The growth cones of chicken retinal neurites are well known to collapse in response to ephrin-A ligand stimulation (Monschau et al. (1997) EMBO Journal 16(6), 1258-1267, Homberger et al. (1999) Neuron 22(4), 731-742). Because EphA4 is homogenously expressed in different parts of the retina, whereas other EphA receptors are preferentially expressed in the temporal but not the nasal region of the retina (Connor et al. (1998) Developmental Biology (Orlando) 193(1), 21-35), EphA4 is the predominant EphA receptor in nasal retinal neurons. Therefore, explants from the chicken nasal retina was used to examine the ability of compounds 1 and 2 to counteract EphA4-mediated growth cone collapse. Although co-expression of ephrin-A ligands with EphA4 in the nasal retina makes the growth cones less sensitive to the collapsing effects of ephrin-A5 Fc, the growth cones still collapse when exposed to high concentrations of the ephrin (Monschau et al. (1997) EMBO Journal 16(6), 1258-1267, Hornberger et al. (1999) Neuron 22(4), 731-742, Connor et al. (1998) Developmental Biology (Orlando) 193(1), 21-35). The KYL peptide, which has been shown to selectively inhibit EphA4-ephrin binding (Murai et al. (2003) Mol Cell Neurosci 24(4), 1000-1011), blocked collapse of nasal growth cones stimulated with ephrin-A5, confirming the requirement for EphA4 activation (FIGS. 8A and 8B). Compound 1 (FIGS. 8C and 8D) and compound 2 (FIGS. 8E and 8F) also blocked the growth cone collapsing effects of ephrin-A5 Fc. Importantly, despite the sensitivity of growth cones to their surrounding environment (Dickson, (2002) Science 298(5600), 1959-1964, Wen and Zheng, (2006) Curr Opin Neurobiol 16(1), 52-58), neither the KYL peptide nor the two compounds at concentrations as high as 400 μM affected the shape of unstimulated growth cones.

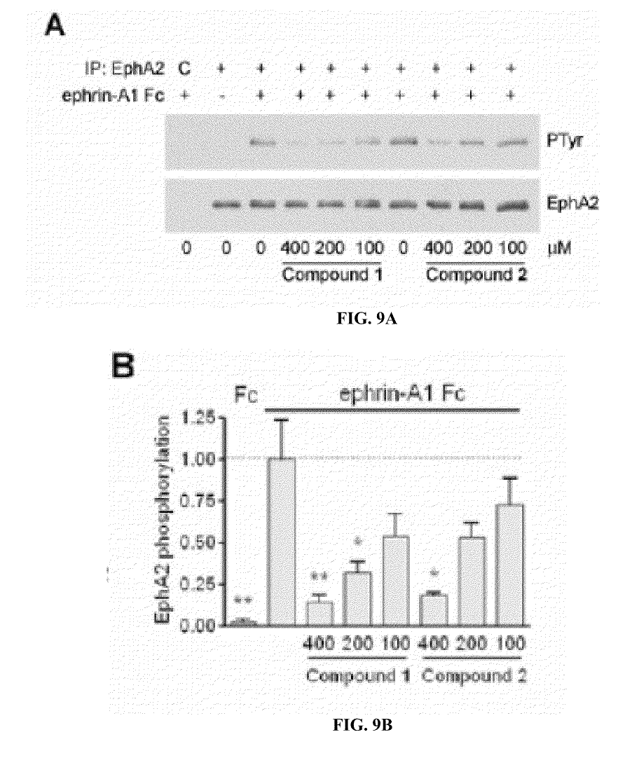

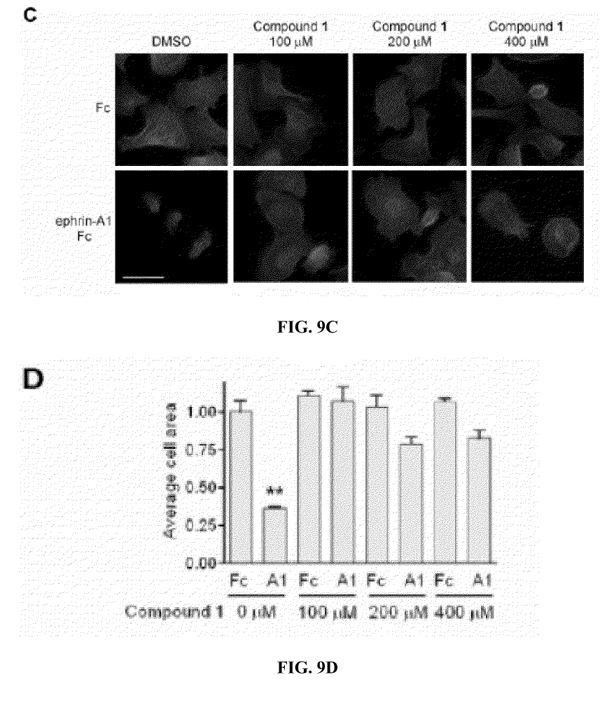

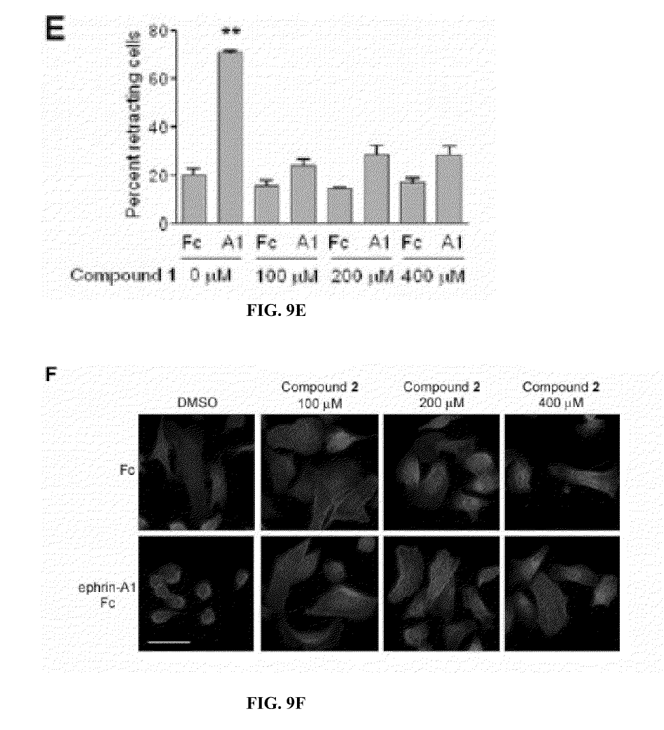

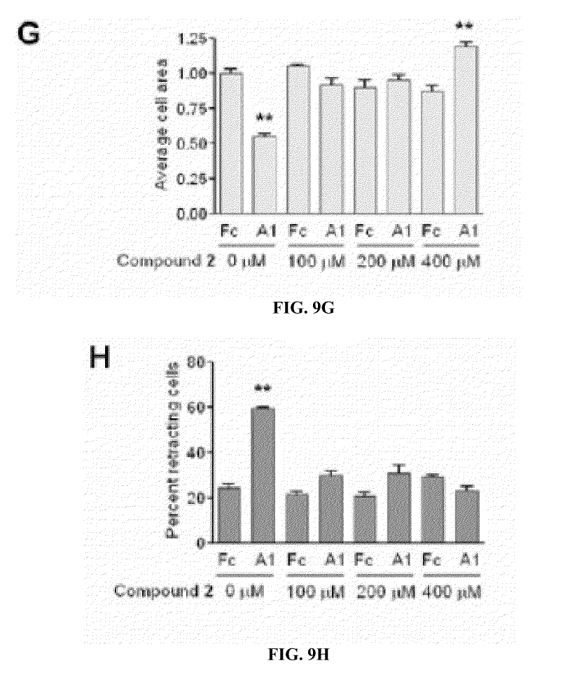

Compounds 1 and 2 Inhibit EphA2-Dependent Retraction of the Cell Periphery—EphA2 is known to induce changes in cell morphology when activated by ephrin-A1, including retraction of the cell periphery and cell rounding (Dail et al. (2006) J Cell Sci 119(Pt 7), 1244-1254; Dail et al. (2006) J Cell Sci 119(Pt 7), 1244-1254). Because EphA2 is the predominant EphA receptor expressed in PC3 prostate cancer cells (Fox et al. (2006) Biochem Biophys Res Commun 342(4), 1263-1272), cells were tested whether compounds 1 and 2 were able to inhibit EphA2-mediated cell retraction. Treatment with the compounds blocked EphA2 activation following stimulation with ephrin-A1 Fc (FIGS. 9A and 9B) as well as the decrease in cell spreading (FIGS. 9C, 9D, 9F and 9G) and the increase in the percentage of rounded cells (FIGS. 9C, 9E, 9F and 9H) caused by ephrin-A1 Fc stimulation. Importantly, the compounds did not affect cell morphology in the absence of ephrin treatment (FIG. 9C-9H).

3. Discussion

The methods and materials described herein are the first reported in which identification of small molecules that can inhibit the interaction between Eph receptors and ephrins is made. In order to isolate small molecule inhibitors of EphA4, a high throughput screening was designed to identify compounds that inhibit ligand binding to this receptor. These kinds of inhibitors are advantageous compared to tyrosine kinase inhibitors because they can act without penetrating inside the cell and can be highly selective. 2,5-dimethylpyrrolyl benzoic acid derivatives was identified to show selectivity for only two Eph receptors: EphA4 and the closely related EphA2. The results also indicate that the two compounds are competitive inhibitors that target the high-affinity ligand binding pocket of the receptors, a conclusion that is supported by NMR studies with EphA4 (Example 2).

Given the small size of the two dimethylpyrrole derivatives compared to the ephrin binding pocket, their selectivity for EphA4 and EphA2 is particularly interesting and surprising and indicates that these compounds target a region that is not highly conserved in other Eph receptors. The two dimethylpyrrole derivatives also show selectivity with regard to ephrin binding, since they inhibited association of most ephrins tested except for ephrin-A4 and ephrin-B2, even when used at high concentrations. This indicates that these ephrins bind differently to the receptors compared to other ephrins of the same class. For example, interfaces not involving the ephrin-binding pocket can be of higher affinity with ephrin-A4 and ephrin-B2 than with other ephrins. Alternatively, there can be differences in the binding of ephrin-A4 and ephrin-B2 to the ephrin-binding pocket despite the similarity of the G-H loops of these ephrins with those of other ephrins whose binding is inhibited by the compounds. Structural studies can elucidate how different ephrins interact with EphA4 and EphA2. The selectivity of the two dimethylpyrrole derivatives towards different Eph receptors and ephrins was confirmed in cell-based assays, where the addition of the compounds selectively blocked the ephrin-dependent tyrosine phosphorylation of EphA4 and EphA2, but not EphB2. The compounds also had no effect on the EGF-dependent phosphorylation of the EGF receptor, which is instead inhibited by many of the small molecules targeting kinase domains (Karaman et al. (2008) Nat Biotechnol 26(1), 127-132) and by epigallocatechin gallate (Liang et al. (1997) J Cell Biochem 67(1), 55-65).

The two pyrrole derivatives, like the KYL peptide, also blocked EphA4-mediated growth cone collapse in retina explants, indicating that the compounds and the KYL peptide can promote axon growth. Interestingly, EphA4 has been proposed to play multiple roles in the inhibition of spinal cord regeneration after injury. In mouse and rat models of spinal cord injury, expression of this receptor is upregulated in both glial cells and neurons near the site of injury (Goldshmit et al. (2004) J Neurosci 24(45), 10064-10073; Fabes et al. (2006) Eur J Neurosci 23(7), 1721-1730). Furthermore, EphA4 expressed in the reactive glial cells can act as a negative regulator of axon regeneration by favoring the formation of the glial scar and by stimulating ephrin-B reverse signaling in axons. EphA4 expressed in the damaged axons can interact with ephrin-B2 expressed in the surrounding astrocytes and ephrin-B3 expressed in myelin, leading to inhibition of axon sprouting and outgrowth (Fabes et al. (2006) Eur J Neurosci 23(7), 1721-1730; Benson et al. (2005) Proc Natl Acad Sci USA 102(30), 10694-10699). Consistent with this, inhibiting EphA4 function can be beneficial for the treatment of spinal cord injuries. For example, it has been reported that EphA4 knock-out mice have a significantly reduced glial scar and improved ability to regenerate spinal cord connections after spinal cord injury (Goldshmit et al. (2004) J Neurosci 24(45), 10064-10073). Furthermore, a recent study has shown that the KYL peptide protects rat neocortical growth cones from collapsing after ephrin-A5 Fc treatment and that infusion of the peptide (Murai et al. (2003) Mol Cell Neurosci 24(4), 1000-1011) into the lesioned spinal cord enhances axon sprouting, reduces cavity formation and improves behavioral recovery (Fabes et al. (2007) Eur J Neurosci 26(9), 2496-2505) Inhibition of retinal growth cone collapse by the two dimethylpyrrole derivatives is an encouraging result that indicates that similar compounds with higher affinity can also be used to enhance axon regrowth after injury. Inhibition of EphA4-ephrin interaction can also be used in neuropathologies characterized by dendritic spine loss in the brain (Murai et al. (2003) Nat Neurosci 6(2), 153-160), to promote blood clotting (Prevost et al. (2005) Proc Natl Acad Sci USA 102(28), 9820-9825), and to inhibit some forms of cancer (Ashida et al. (2004) Cancer Res 64(17), 5963-5972; Iiizumi et al. (2006) Cancer Sci 97(11), 1211-1216; Yamashita et al. (2008) J Biol Chem).

The other Eph receptor targeted by the two dimethylpyrrole derivatives, EphA2, is widely expressed in many types of cancer cells and in the tumor vasculature (Ireton and Chen, (2005) Curr Cancer Drug Targets 5(3), 149-157; Landen, C. N., Kinch and Sood, (2005) Expert opinion on therapeutic targets 9(6), 1179-1187; Brantley-Sieders and Chen, (2004) Angiogenesis 7(1), 17-28). The dimethylpyrrole derivatives inhibit EphA2-dependent retraction and rounding of prostate cancer cells stimulated with exogenous ephrin-A1 Fc, indicating that treatment with the compounds can inhibit the functional effects of EphA2. Interestingly, the compounds completely reverted the effect of ephrin-A1 treatment on cell retraction and rounding at concentrations that only partially inhibited EphA2 tyrosine phosphorylation, indicating that high levels of EphA2 activation can be required to promote changes in cell adhesion and morphology. Inhibiting EphA2-ephrin binding in cancer cells will be useful in the cases where EphA2 is highly activated and its signaling activity promotes tumorigenesis (Brantley-Sieders et al. (2006) Cancer Res 66(21), 10315-10324; Hess et al. (2001) Cancer Research 61(8), 3250-3255; Hess et al. (2006) Cancer Biol Ther 5(2), 228-233), but not in others where the tumor cells express low levels of endogenous ephrin-A1 (Macrae et al. (2005) Cancer Cell 8(2), 111-118). However, the most exciting application of EphA2-targeting molecules is for inhibition of tumor angiogenesis and other forms of pathological angiogenesis (Brantley et al. (2002) Oncogene 21(46), 7011-7026; Cheng, N et al. (2002) Mol Cancer Res 1(1), 2-11; Brantley-Sieders et al. (2004) J Cell Sci 117(Pt 10), 2037-2049; Brantley-Sieders et al. (2005) Faseb J 19(13), 1884-1886; Hunter et al. (2006) Mol Cell Biol 26(13), 4830-4842; Chen et al. (2006) Exp Eye Res 82(4), 664-673; Baldwin et al. (2006) Am J Physiol Renal Physiol 291(5), F960-971) Importantly, EphA2 is expressed in adult angiogenic blood vessels, but not in embryonic or adult quiescent blood vessels (Brantley-Sieders and Chen, (2004) Angiogenesis 7(1), 17-28; Ogawa et al. (2000) Oncogene 19(52), 6043-6052), consistent with evidence that targeting the pathological effects of EphA2 does not affect the normal vasculature. Unlike the previously identified EphA2-targeting peptides, which inhibit EphA2-ephrin binding in ELISA assays but stimulate EphA2 phosphorylation in cells (Koolpe et al. (2002) J Biol Chem 277(49), 46974-46979), the dimethylpyrrole derivatives also inhibit EphA2 activation in cells, including endothelial cells treated with the angiogenic factor TNFα (Pandey et al. (1995) Science 268(5210), 567-569). Thus, this class of compounds can be used for inhibition of pathological forms of angiogenesis, similar to the EphA receptor Fc fusion proteins that have been successfully used to inhibit angiogenesis in mouse tumor models and in a rat model of retinopathy of prematurity (Brantley et al. (2002) Oncogene 21(46), 7011-7026; Cheng et al. (2002) Mol Cancer Res 1(1), 2-11; Chen et al. (2006) Exp Eye Res 82(4), 664-673; Cheng et al. (2003) Neoplasia 5(5), 445-456).

In order to obtain compounds with improved potency, the structure-activity relationship of 49 analogs of the dimethylpyrrole derivatives was examined. Although none of these compounds showed measurable inhibition of EphA4-ephrin-A5 binding, the IC₅₀ values obtained with the KYL peptide provided information on the structural features important for inhibition of ligand binding to EphA4. The results indicate that the hydroxyl and carboxylic groups on the benzene ring as well as the dimethylpyrrole ring are all necessary for the activity of the compounds. Therefore, three additional analogs containing these structural elements were designed.

The first two, compounds 3 and 4, have the carboxylic acid on the benzene ring located at the end of aliphatic chains in their acetic or propanoic acid groups, respectively. Although the presence of a propanoic acid (compound 5) or acetic acid (compound 6) in place of the carboxylic acid (compound 25) as the only substituent on the benzene ring greatly improved inhibition of EphA4-KYL binding, when the hydroxyl group was also present compounds 3 and 4 with the aliphatic chains were somewhat less active compared to compound 1 with the carboxylic acid. The hydroxyl group highly improved the ability of compounds 3 and 4 to inhibit ephrin-A5 binding compared to compounds 6 and 5, which did not show any activity with ephrin-A5. However, inhibition of EphA4-KYL binding was not greatly affected, indicating that the presence of the hydroxyl group is important for inhibition of ephrin-A5 binding but not peptide binding. This was confirmed by the lack of activity with ephrin-A5 of compound 7, a methyl ether of compound 4, which however inhibited EphA4-KYL binding with a low IC₅₀ value. It is also interesting that despite being able to inhibit ephrin-A5 binding to EphA4, compound 3 inhibited EphA4-KYL binding less effectively than compounds 5, 6 and 7, which do not measurably inhibit ephrin binding. This supports the idea that somewhat different structural features can be required for inhibition of EphA4 interaction with ephrin-A5 versus the KYL peptide.

The third rationally designed compound that was synthesized, compound 19, corresponds to compound 2 with an additional methyl group as a substituent on the benzene ring. The lack of inhibitory activity of this compound with ephrin-A5 and its very low activity with the KYL peptide were unexpected. Perhaps the ability of the methyl group to enhance the activity of compounds 10 and 15 depends on its position with respect to the other substituents on the benzene ring. If this is true, the synthesis of alternative compounds carrying the methyl group at different positions can give different results. The rational design of other analogs with improved potency should now be possible based on the three-dimensional structure of EphA4 in complex with compounds 1 and 2, which provides valuable insight into the molecular interactions of the compounds with the receptor (see Example 2).

### D. Example 2

### Structural Analysis of EphA4 Inhibitor Binding to EphA4

This example describes a structure of the high affinity binding site in EphA4 and EphA2. The structural identification of the high affinity binding site can explain the selectively of the two exemplary isomeric 2,5-dimethylpyrrolyl benzoic acid compounds to the inhibition of ephrin binding to EphA4 and EphA2 as well as the functions of these receptors in live cells. The structural understanding of the high affinity binding pocket can be used to design and/or validate additional EphA2/4 inhibitors.

### 1. Material and Methods

Cloning, expression and purification of the EphA4 ligand-binding domain—The DNA fragment encoding the human EphA4 ligand-binding domain (residues 28-208) was amplified from a Hela cell cDNA library by using two primers containing BamHI and XhoI restriction sites: 5′-GGATCCAATGAAGTTACCTTATTGGATTCC-3′ (SEQ ID NO:1)(forward) and 5′-CTCGAGTCAGCGGACTGTGAGTGGACAC-3′ (SEQ ID NO:2)(reverse). The PCR fragment was cloned into a modified pET32a vector (Novagen) and the vector was transformed into E. coli Rosetta-gami (DE3) cells (Novagen) as previously described (Ran et al. (2008) Proteins. 72, 1019-29), allowing more efficient formation of disulfide bonds and expression of eukaryotic proteins containing codons rarely used in E. coli. To enhance the solubility of the EphA4 ligand-binding domain for NMR studies, in this construct a C-terminal tail (residues 175-181) was also included, which was found to be totally unstructured in all structures determined so far. The free Cys176 in this extra tail was mutated to Ala by use of the site-directed mutagenesis kit (Stratagene) to avoid the formation of non-native disulfide bridges.

The cells were cultured in Luria-Bertani medium at 37° C. until the absorbance at 600 nm reached ˜0.7. Then 0.4 mM isopropyl-1-thio-d-galactopyranoside (IPTG) was then added to induce EphA4 expression at 20° C. overnight. The harvested cells were sonicated in the lysis buffer containing 150 mM sodium chloride, 20 mM sodium phosphate, pH 7.2 to release soluble His-tagged proteins, which were subsequently purified by affinity chromatography using Ni-NTA agarose (Qiagen). In-gel cleavage of the EphA4 fusion protein was performed at room temperature by incubating the fusion protein attached to Ni-NTA agarose with thrombin overnight. The released EphA4 protein was further purified on a AKTA FPLC machine (Amersham Biosciences) using a gel filtration column (HiLoad 16/60 Superdex 200) equilibrated with a buffer containing 150 mM NaCl, 50 mM Tris-HCl, pH 7.5, followed by ion-exchange chromatography on an anion-exchange column (Mono Q 5/50). The eluted fraction containing the EphA4 ligand-binding domain was collected and buffer-exchanged to a buffer containing 150 mM NaC1 25 mM Tris-HCl, and 5 mM CaCl₂, pH 7.8 for storage.

The generation of the isotope-labeled proteins for NMR studies followed a similar procedure except that the bacteria were grown in M9 medium with the addition of (15NH₄)₂SO₄ for ¹⁵N labeling and (15NH₄)₂SO₄[¹³C]-glucose for ¹⁵N-/¹³C-double labeling (Ran et al. (2008) Proteins. 72, 1019-29; Ran and Song, (2005) J. Biol. Chem. 280, 19205-12). The purity of the protein samples was verified by the SDS-PAGE gel, and the molecular weight of the recombinant EphA4 ligand-binding domain was verified by a Voyager STR MALDI-TOF mass spectrometer (Applied Biosystems). The concentration of protein samples was determined by use of a previously-described spectroscopic method in the presence of denaturant (Pace et al. (1995) Protein Sci. 4, 2411-23).

Crystallization, data collection and structure determination—The EphA4 ligand-binding domain was prepared at a concentration of 12 mg/ml and crystallized by setting up 2 μl hanging drops at room temperature in well containing the reservoir solution (20% PEG 4000, 15% isopropanol and 0.1 M Hepes at pH 7.5). Rock-like crystals formed after 4 days and dehydration of the crystals was subsequently performed by moving the coverslips to a new well containing dehydration buffer (20% PEG 4000, 15% isopropanol, 10% glycol and 0.1 M Hepes at pH 7.5).

The X-ray diffraction images for a single crystal were collected by using an in-house Rigaku/MSC FR-E X-ray generator with an R-AXIS IV++ imaging plate detector at the Biopolis shared-equipment facility. The crystal was protected by the cryoprotectant (20% PEG 4000, 15% isopropanol, 25% glycol and 0.1 M Hepes at pH 7.5). The data were indexed and scaled using the program d*Trek (Shi et al. (2008) J. Virol. 82, 4620-4629; Otwinowski and Minor, (1997). In C. W. Carter, Jr., and R. M. Sweet (ed.), Methods in enzymology, 276, 307-326. Academic Press, New York, N.Y.). After an all-space-group-search, the crystal was identified to belonging to the space group P22₁2₁ with a=53.75, b=71.12 and c=127.00 with two molecules per asymmetric unit. The Matthews coefficient was 2.91 with 57.68% solvent constant.

The initial model of the EphA4 ligand-binding domain was generated by the program Phaser from the Phenix suite (McCoy et al. (2005). Acta Crystallogr. D 61:458-464) using the EphB2 structure (1NUK) as a search model through the molecular replacement method. This model was completed by manual fitting using the program COOT (Emsley and Cowtan, (2004). Acta Crystallogr. D 60:2126-2132), and refined using the program Phenix for many rounds (Adams et al. (2002) Acta Cryst. D58, 1948-1954). During model building and refinement, 9.11% of the data was reserved for cross validation to monitor the refinement progress. The final R-factor was 0.2335 (Rfree=0.2869) at 2.8 Å resolution. The final structure was analyzed by PROCHECK (Laskowski et al. (1993). J. Appl. Cryst. 26:283-291) and details of the data collection and refinement statistics are shown in Table 1. The atomic coordinates were deposited in the Protein Data Bank with the PDB ID (3CKH). Figures showing the structure were prepared using the Pymol molecular graphics system (W. L. DeLano, DeLano Scientific LLC, San Carlos, Calif.).

TABLE 1
Crystallographic data and refinement statistics for the EphA4 ligand-
binding domain structure

| Data Collection | |
|---|---|
| Wavelength (Å) | 1.5418 |
| Resolution limit (Å) | 63.52-2.80 (2.90-2.80) |
| Space group | P 22₁2₁ |
| Cell parameters | |
| a (Å) | 53.75 |
| b (Å) | 71.12 |
| c (Å) | 127.00 |
| Observed reflections | 93170 |
| Unique reflections | 12572 |
| Completeness | 99.7% (99.7%) |
| Redundancy | 7.41 (7.52) |
| Linear R-factor | 0.087 (0.395) |
| Overall I/(I) | 11.6 (3.5) |
| Refinement | |
| Resolution range (Å) | 19.70-2.80 (2.90-2.80) |
| R_{work}** | 0.233 (0.305) |
| Number of Reflections | 11229 |
| R_{free}*** | 0.286 (0.371) |
| Number of reflections | 1126 |
| Rmsd bond lengths (Å) | 0.007 |
| Rmsd bond angles (deg) | 1.17 |
| Ramachandran plot | |
| Favored, % | 83.0 |
| Allowed, % | 16.7 |
| Generously allowed, % | 0.3 |
| Disallowed, % | 0 |

*Values in parenthesis are for highest-resolution shell.
**R_{work} = Σ|F_{obs} − F_{calc}|/ΣF_{obs} where F_{calc} and F_{obs} are the calculated and observed structure factor amplitudes, respectively.
***R_{free} = as for R_{work}, but for 9.11% of the total reflection chosen at random and omitted from refinement.

Oligomerization status characterized by FPLC dynamic light scattering and analytic ultracentrifugation—The oligomerization status of the EphA4 ligand-binding domain was assessed by FPLC gel-filtration, dynamic light scattering, as well as analytic ultracentrifugation in solution. Briefly, as previously described (Ran, X., Qin, H., Liu, J., Fan, J S., Shi, J., and Song, J. (2008) Proteins. 72, 1019-29), the FPLC gel filtration experiments were conducted using a fast protein liquid chromatography AKTA instrument (Amersham Biosciences) with a gel filtration column (HiLoad 16/60 Superdex 200). The column was calibrated with a low molecular weight protein kit (Amersham Biosciences) including four proteins: ribonuclease A (15.6 kDa), chymotrypsinogen A (22.8 kDa), ovalbumin (48.9 kDa), and albumin (65.4 kDa). Dynamic light scattering experiments were performed at 20° C. on a DynaPro-MS/X instrument (Protein Solutions Inc.) and the apparent molecular mass values were calculated from 10 readings using the Protein Dynamics analysis software (Shi et al. (2008) Biomaterials. 29, 2820-2828). Sedimentation velocity experiments were done at 20° C. using a Beckman Coulter XL-I analytical ultracentrifuge as previously described (Shi et al. (2008) J. Virol. 82, 4620-4629).

Binding characterization by isothermal titration calorimetry and circular dichroism—Isothermal titration calorimetry experiments were performed using a Microcal VP ITC machine as previously-described (Liu et al. (2006) Biochemistry 45, 7171-84). Titrations were conducted in 10 mM phosphate buffer (pH 6.3) at 25° C. The two small molecule antagonists were purchased from Matrix Scientific, with 4-(2,5-dimethyl-pyrrol-1-yl)-2-hydroxy-benzoic acid being designated as compound 1 and 5-(2,5 dimethyl-pyrrol-1-yl)-2-hydroxy-benzoic acid as compound 2. The powders of the two compounds were weighted and then dissolved in 10 mM phosphate buffer with the final pH values adjusted to 6.3. The EphA4 receptor at a concentration of 70 μM was placed in a 1.8 ml sample cell while the compounds at a concentration of 2 mM were loaded into a 300 μL syringe. The samples were degassed for 15 min to remove bubbles before the titrations were initiated. Control experiments with the same parameter settings were also performed for the two compounds without EphA4, to subtract the effects due to sample dilution. To obtain thermodynamic binding parameters, the titration data after subtracting the values obtained from the control experiments were fit to a single binding site model using the built-in software ORIGIN version 5.0 (Microcal Software Inc.). The detailed set-up and the results are documented in Table 2.

TABLE 2
Thermodynamic parameters for the binding interactions
between EphA4 and two small molecules by ITC

| | | Injection | Kₐ | K_{b} | | ΔS | ΔH |
|---|---|---|---|---|---|---|---|
| Syringe | Cell | Volume (μL) | (M⁻¹) | (μM) | Stoichiometry | (cal/mol*K) | (cal/mol*K) |
| Comp. 1 | EphA4 | 5 | 4.893 × 10⁴ ± | 20.44 | 1.000 ± 0 | 18.11 | −1.001 ± 0.027 |
| (2 mM) | (70 μM) | | 5071 | | | | |
| Comp. 2 | EphA4 | 5 | 3.785 × 10⁴ ± | 26.42 | 1.000 ± 0 | 20.15 | −0.237 ± 0.013 |
| (2 mM) | (70 μM) | | 7575 | | | | |

Comp. 1: 4-(2,5-Dimethyl-pyrrol-1-yl)-2-hyrdoxyl-benzoic acid
Comp. 2: 5-(2,5-Dimethyl-pyrrol-1-yl)-2-hyrdoxyl-benzoic acid

The samples were prepared for circular dichroism experiments (CD) by buffer exchanging the EphA4 ligand-binding domain into a 10 mM phosphate buffer (pH 6.3) at a protein concentration of 20 μM. The far-UV circular dichroism experiments were performed using a Jasco J-810 spectropolarimeter and data from five independent scans were averaged (Liu, J., Li, M., Ran, X., Fan, J S., and Song, J. (2006) Biochemistry 45, 7171-84). The spectra of the EphA4 receptor in the absence or in the presence of the two compounds at a molar ratio of 1:6 (EphA4:compounds) were collected at room temperature. The contribution of the two compounds and the buffer was removed by subtracting the CD spectra of the two compounds diluted at the identical concentrations and in the same buffer.

Binding characterization by NMR—Samples were prepared for NMR experiments in 10 mM phosphate buffer (pH 6.3), with the addition of 10% D₂O for NMR spin-lock. All NMR data were collected at 25° C. on an 800 MHz Bruker Avarice spectrometer equipped with a shielded cryoprobe as previously described (Ran et al. (2008) Proteins. 72, 1019-29; Ran and Song, (2005) J. Biol. Chem. 280, 19205-12; Liu et al. (2006) Biochemistry 45, 7171-84; Sattler et al. (1999) Prog. NMR Spectrosc. 34, 93-158). For the preliminary sequential assignment, a pair of triple-resonance NMR spectra: HNCACB and CBCA(CO)NH, were acquired on a double-labeled EphA4 sample at a concentration of 500 μM. The obtained sequential assignments were further confirmed by analyzing other three-dimensional spectra including (H)CC(CO)NH, H(CCO)NH, and ¹⁵N-edited HSQC-TOCSY, HSQC-NOESY and ¹³C-edited HCCH-TOCSY and NOESY. All NMR data were processed with NMRPipe (Delaglio et al. (1995) J Biomol. NMR 6, 277-293) and analyzed with NMRView (Johnson and Blevins, (1994) J. Biomol. NMR 4, 603-614).

For NMR characterization of the binding of the EphA4 ligand-binding domain with two small molecules, two-dimensional ¹H-¹⁵N HSQC spectra were acquired at a protein concentration of 100 μM in the absence of or in the presence of the two molecules at different molar ratios including 1:1; 1:2, 1:4, 1:6, 1:8 (EphA4:compounds). By superimposing the HSQC spectra, the shifted HSQC peaks could be identified and further assigned to the corresponding EphA4 residues (Liu et al. (2006) Biochemistry 45, 7171-84). The degree of perturbation was reflected by an integrated index calculated by the formula [(Δ¹H)²+(Δ¹⁵N)²/5]^{1/2}. The interactions were investigated by monitoring the line-broadening and shifting of the resonance peaks of the two compounds in their one-dimensional NMR spectra upon the progressive addition of the EphA4 protein.

Molecular docking—The models of the EphA4 ligand-binding domains in complex with two antagonistic molecules were constructed by use of the HADDOCK software (Dominguez et al. (2003). J. Am. Chem. Soc. 125, 1731-1737; de Vries et al. Proteins. 69, 726-733 (2007)) in combination with CNS (Brunger et al. (1998). Acta Crystallogr. D 54:905-921), which makes use of chemical-shift perturbation data to derive the docking while allowing various degrees of flexibility. The docking procedure was performed by three steps: first, randomization and rigid body energy minimization; second, semi-flexible simulated annealing; third, flexible explicit solvent refinement.

To conduct the docking, several invisible residues over the loop regions were added to the EphA4 crystal structures by COOT (Emsley and Cowtan, (2004). Acta Crystallogr. D 60:2126-2132) and then the obtained structures were subjected to several rounds of energy minimization by PHENIX (Adams et al. (2002) Acta Cryst. D58, 1948-1954). Subsequently, hydrogen atoms were added to the structures by use of the CNS protocol. On the other hand, the geometric coordinates and parameters for the two small molecules were generated and energy-minimized by the on-line PRODRG server (Schuettelkopf and van Aalten, (2004). Acta Crystallographica D60, 1355-1363).

All EphA4 residues with a chemical shift perturbation greater than the threshold value of 0.08 (2.5 times of the standard deviation) were set to be “active” residues (Zhang et al. (2006) J Mol. Biol. 363, 188-200) while neighbors of active residues were defined as “passive residues” according to HADDOCK definition. These active residues included Gln43 on the E β-strand, Ile31-Met32 and Ile39 on the D-E loop, and Asp123 and Ile131-Gly132 on the J-K loop. Furthermore, all residues with heteronuclear NOE intensities of less than 0.7 were found to be located on the N- and C-termini, or on the loops, and thus set to be “fully-flexible” during the molecular docking. One thousand structures were generated during the rigid body docking, and the best 50 structures were selected for semi-flexible simulated annealing, followed by water refinement. Three structures with the lowest energies were selected for detailed analysis and display.

### 2. Results

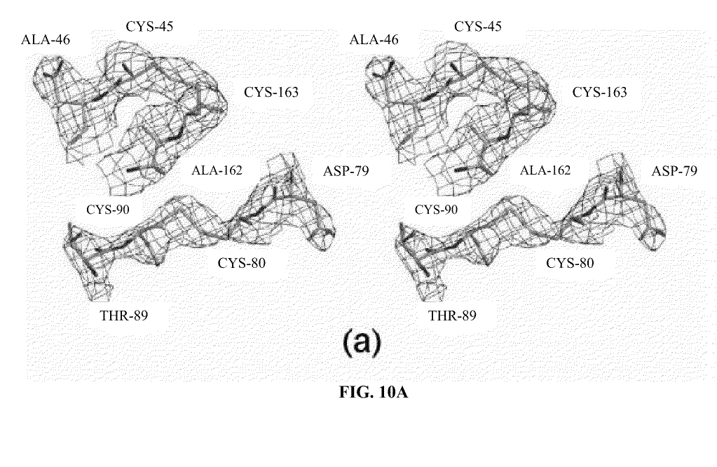

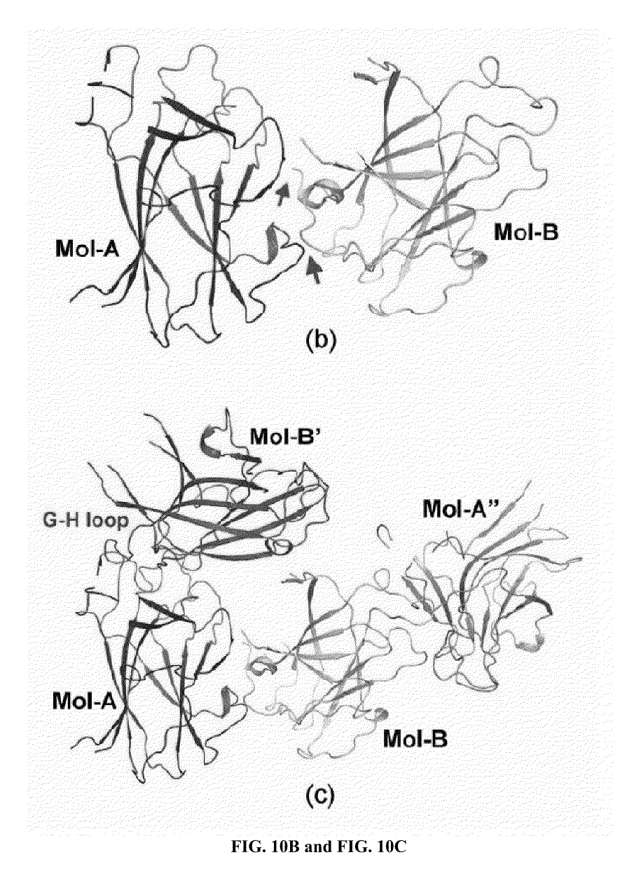

Structure determination—The EphA4 ephrin-binding domain was successfully crystallized without a bound ligand, allowing determination of the crystal structure at 2.8 Å resolution with a final R-factor of 0.2335 (Rfree=0.2869). Details of the data collection and refinement statistics are summarized in Table 1. In the final model, one asymmetric unit contains two EphA4 molecules designated as A and B (FIG. 10). Due to poor electron density, probably resulting from the inherent flexibility in the absence of bound ligand, some residues were invisible. These residues included the C-terminal seven residues (175-181) for both molecules; Met32, Thr37, Pro38 and Asp133 for molecule A and Met32-Asn36, Ile131-Gly132 for molecule B.

As seen in FIG. 10A, there are two conserved disulfide bridges in the EphA4 ligand-binding domain, one within the G-H loop (Cys80-Cys90) and the other between the E-F and L-M loops (Cys45-Cys163). This pattern of disulfide bonds is identical to that observed in the EphB2 and EphB4 structures (Himanen et al. (1998) Nature 396, 486-491; Chrencik et al. (2006) J Biol. Chem. 281, 28185-92). Interestingly, the two EphA4 molecules appear to pack against each other to form an asymmetric dimer with an interface not observed previously with other Eph receptors, involving residues Ile18-Pro20 and Arg107-Glu111 of molecule A and Val3-Val11 of molecule B (FIG. 10B). Moreover, the two EphA4 molecules in one asymmetric unit pack differently with other EphA4 molecules in neighboring units. The high-affinity ligand binding channel of molecule A appears partly occupied by the G-H loop of molecule B′ in a neighboring asymmetric unit, while the G-H loop of molecule B inserts into the high-affinity ligand binding channel of molecule A″ in another neighboring asymmetric unit (FIG. 10C). Likely owing to this differential packing interactions with other EphA4 molecules in neighboring asymmetric units, molecules A and B in the same asymmetric unit show some structural differences over the D-E and J-K loops.

As shown in FIG. 11A, EphA4 molecules A and B adopt the conserved jellyroll folding architecture previously revealed for the EphB2 and EphB4 receptors, composed of 11 antiparallel β-sheets arranged as a compact β-sandwich. The concave sheet is comprised of strands C, F, L, H, and I, and the convex sheet of strands D, E, A, M, G, K, and J, which are connected by loops of variable length. If only the 11β-strands are superimposed, the rmsd deviations between the EphA4 A and B molecules are only 0.074 Å for all atoms and 0.062 Å for backbone atoms. However, molecules A and B have marked differences over the D-E and J-K loops, which are the key components of the high-affinity ephrin-binding channel. Without considering D-E and J-K loop residues Met32-Ile39 and Asp123-Leu138, the rmsd deviation between the A and B structures is only 0.4 A for all atoms. The most distinguishable difference between the A and B molecules involves the J-K loop. The four residues Phe126-Val129, which adopt no regular secondary structure in molecule A, form a short β-strand in molecule B that packs against the extended K-strand residues Met 136-Asn139.

As shown in FIG. 11B, despite belonging to the EphA subclass, the structure of the EphA4 ligand-binding domain bears a high similarity over the 11 β-stranded regions to the previously determined ligand-binding domains of the EphB2 and EphB4 receptors. The backbone rms deviations of the EphA4 ligand-binding domain over 11 β-strands are 1.05 Å compared to EphB2 in the free state (1NUK), 1.07 Å compared to EphB2 in complex with ephrin-B2 (1KGY), 0.74 Å compared to EphB2 in complex with ephrin-A5 (1SHW), 0.70 Å compared to EphB2 in complex with an antagonistic peptide (2QBX), 0.79 Å compared to EphB4 in complex with an antagonistic peptide (2BBA) and 0.80 Å compared to EphB4 in complex with ephrin-B2 (2HLE). On the other hand, very large variations axe observed over the loop regions not only between EphA4 and the EphB receptors, but also between EphB receptors, in particular over the D-E and J-K loops, which are critical for ligand binding. Indeed, the structural flexibility of these loops has been well demonstrated in previously-determined EphB structures. Interestingly, the additional short β-sheet observed in the J-K loop of molecule B of the EphA4 ligand binding domain was also observed in the structure of EphB2 in complex with an antagonistic peptide (2QBX) (Chrencik et al. (2007) J Biol. Chem. 282, 36505-13). In addition, the EphB receptors have a 4-residue insert in the H-I loop, which is absent in the EphA receptors. While the H-I loop has no regular secondary structure in all the EphB receptor structures examined, the H-I loop of the EphA4 receptor is shorter and residues Glu111-Asn112-Gln113 form a 3₁₀-helix (See FIGS. 11A and 11B).

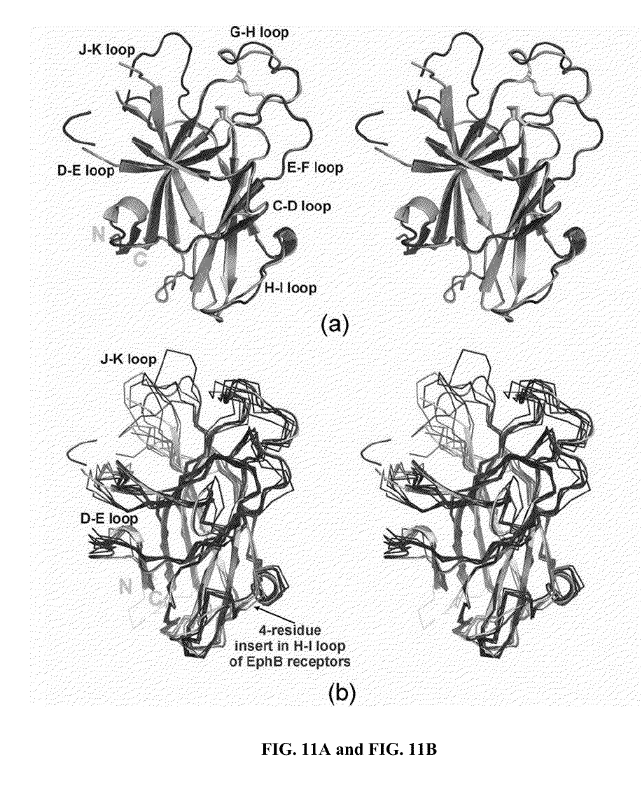

During the preparation of this manuscript, the crystal structure of the EphA2 ligand-binding domain was released by a structural genomics consortium (3C8X). The EphA2 crystals have only one molecule in each asymmetric unit and structural comparison shows that the two EphA4 molecules and EphA2 are highly similar over the 11 β-stranded regions (only ˜0.45 A for the backbone rms deviations) and have identical patterns of disulfide bridges (FIG. 12). Additionally, the short 3₁₀-helix observed in the H-I loop of EphA4 is also presented in EphA2. Nevertheless, some structural variations exist over the H-I, G-H and particularly D-E and J-K loops. Although most J-K loop residues (149-159) are completely missing in the EphA2 structure, structural superimposition indicates that the J-K loop of EphA2 is more similar to that of the EphA4 molecule B (FIG. 12). This observation indicates that EphA4 molecule B can have more properties of the free state while EphA4 molecule A can be more close to the ligand-bound conformation because its ligand-binding channel is partly occupied by the G-H loop of the neighboring EphA4 molecules in the other asymmetric unit.

The oligmerization state of the EphA4 ligand-binding domain was assessed in solution by use of FPLC gel filtration, dynamic light scattering, and analytical ultracentrifugation. The EphA4 ligand-binding domain was constantly eluted as a monomer on a FPLC gel filtration column, even at concentrations of up to 12 mg/ml (HiLoad 16/60 Superdex 200). Dynamic light scattering, and analytical ultracentrifugation data also indicate that the EphA4 ligand-binding domain exists in a monomeric state in solution at concentrations of approximately 100 μM. Therefore, the EphA4 dimerization observed in the same asymmetric unit and the interactions among EphA4 molecules in the different units likely result from the packing force in the crystals.

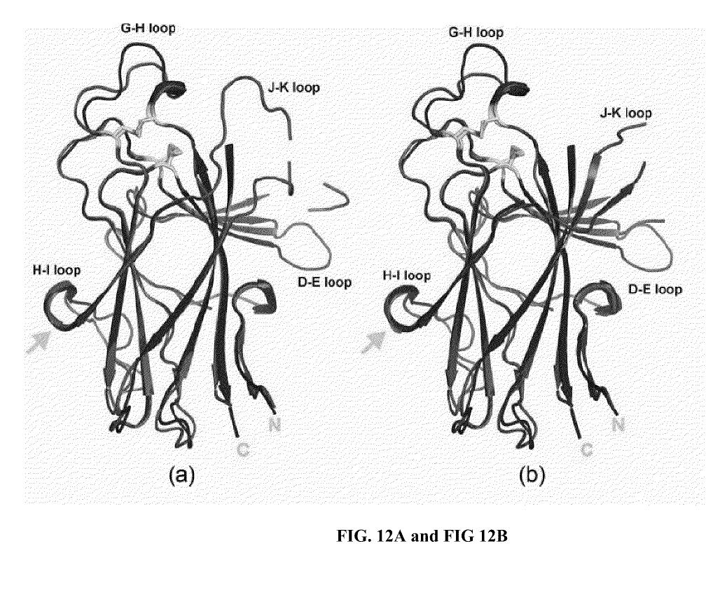

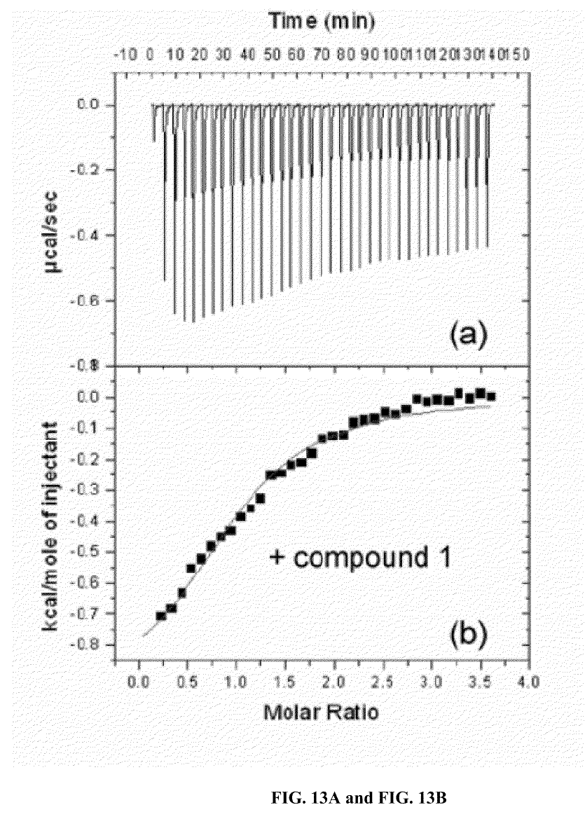

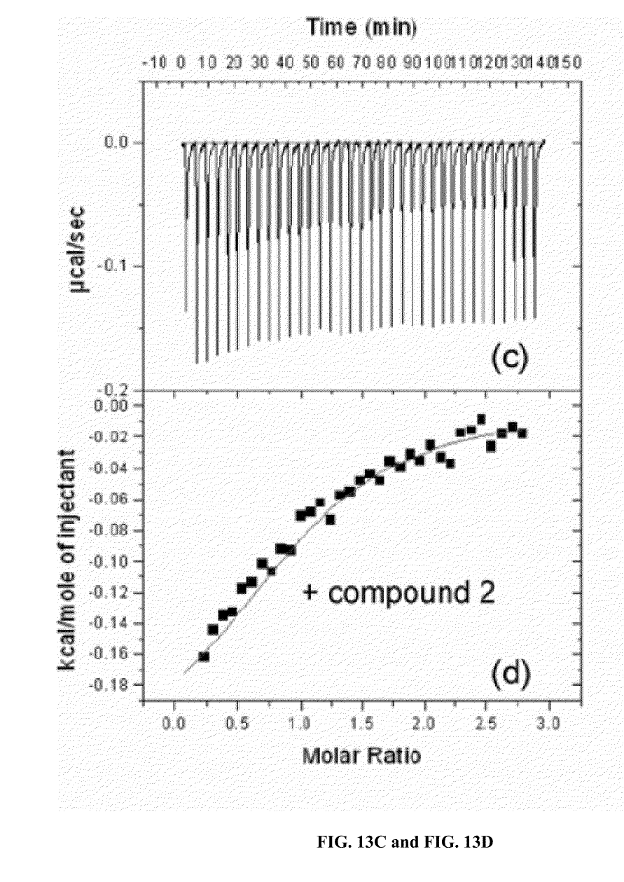

Binding interactions characterized by isothermal titration calorimetry and circular dichroism—Recently, a 2,5-dimethylpyrrolyl benzoic acid derivative has been identified in a high throughput screening for inhibitors of EphA4 ligand binding (see Example 1). This small molecule and an isomeric compound were found to antagonize ephrin-induced effects in EphA4-expressing cells. To assess whether the two isomeric small molecules directly interact with the EphA4 ligand-binding domain, isothermal titration calorimetry was utilized to measure their thermodynamic binding parameters. By using a high concentration of the EphA4 ligand-binding domain (70 μM), these parameters could be obtained (FIG. 13 and Table 2), thus clearly confirming that the two small molecules do interact with the ligand-binding domain of EphA4. Interestingly, the two compounds have similar binding affinities for the EphA4 ligand-binding domain (Kd values of 20.4 μM for compound 1 and 26.4 μM for compound 2), but their binding causes different enthalpy changes (ΔH values of −1,001 for compound 1 and −237 cal for compound 2).

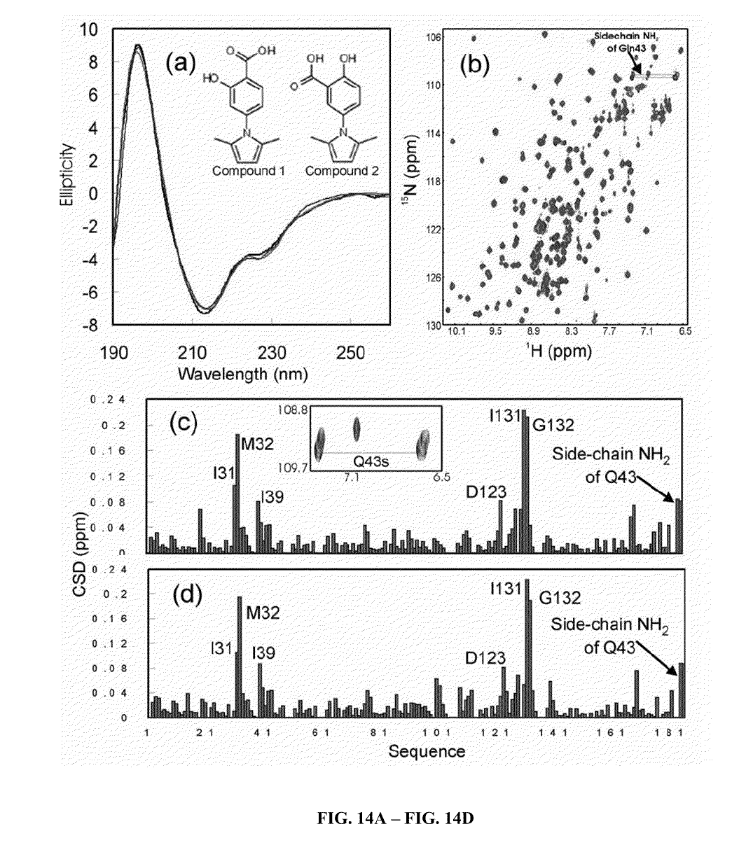

Far-UV circular dichroism (CD) spectroscopy was also used to monitor the overall structural changes in the EphA4 ligand-binding domain upon binding of the two molecules. As seen in FIG. 14A, no significant difference was detected between the far-UV CD spectra of EphA4 in the absence and in the presence of the two small molecules at a molar ratio of 1:6 (EphA4:compound). This result indicates that no significant changes in secondary structure occurred in the EphA4 ligand-binding domain upon binding, which is consistent with the relatively weak binding affinity of the two molecules.

Binding interactions characterized by NMR— Because the two small molecules have medium binding affinity for EphA4, it would be difficult to obtain stable receptor-compound complexes for co-crystallization. It was therefore decided to probe their binding interactions with EphA4 using NMR spectroscopy, which is highly sensitive to weak binding. ¹⁵N/¹³C double-labeled EphA4 was prepared, a series of three-dimensional heteronuclear NMR spectra were collected at a protein concentration of 500 μM, and completed the sequential assignments. As evident from the very large dispersions in both dimensions (3.7 ppm for ¹H and 25 ppm for ¹⁵N) of the HSQC spectrum (FIG. 14B), the EphA4 ligand-binding domain is well-folded in solution. Only one set of HSQC peaks was observed for all the EphA4 residues, indicating that the asymmetric dimer observed in the crystals does not exist in solution on the NMR time scale.

Subsequently NMR HSQC titrations were used to detect the EphA4 residues that were perturbed by the binding of two compounds. Since the chemical shift value of a NMR active atom is sensitive to its chemical environment, chemical shift perturbation analysis upon titration of ligands represents a powerful method for identifying residues that directly contact the ligands or that are indirectly affected by the binding event. Two-dimensional ¹H-¹⁵N HSQC spectra of 15N-labeled EphA4 were recorded to monitor the changes of the HSQC cross-peaks of the amide groups induced by successive additions of the two compounds. A gradual shift of the EphA4 HSQC peaks was observed, correlating with the increased concentrations of the two compounds, which indicates that the free and bound EphA4 molecules undergo a fast exchange on the chemical shift timescale. This allowed assignment of the resonances in the complex by following the shifts in the EphA4 cross-peaks upon gradual addition of increasing amounts of two compounds.

As shown in the isothermal titration calorimetry profiles (FIG. 13), the binding interaction of EphA4 with the two compounds was largely saturated at molar ratios beyond 1:4 (EphA4: compound). Consistent with this, many HSQC peaks did not exhibit significant further shifts at molar ratios beyond 1:6. Therefore, to identify the interaction surfaces, the chemical shift differences (CSD) between the free state and the bound state in the presence of a 6-fold excess of the two compounds were calculated as described in Materials and Methods and plotted versus the EphA4 sequence (FIGS. 14C and 14D). The two compounds induced similar shift patterns for the EphA4 residues and most EphA4 residues did not experience large chemical-shift perturbations, indicating that the two compounds did not alter the overall structure of EphA4, consistent with the circular dichroism results shown in FIG. 14A. The NMR sequential assignments was also completed for the EphA4 ligand-binding domain in the absence and in the presence of compound 1, confirming that binding of this compound does not induce significant changes in the secondary structure of EphA4. Interestingly, only 8 resonance peaks with significant CSD (deviating more than 2.5 standard deviations from the mean CSD) were observed, including residues Ile31-Met32 and Ile39 located in the D-E loop, Gln43 in the E β-strand, and Asp123 and Ile131-Gly132 in the J-K loop. Since the E β-strand and the D-E and J-K loops have been previously shown to be key components of the high-affinity ephrin-binding channel of the Eph receptors, the NMR titration results thus suggest that the two molecules bind to the high-affinity ephrin-binding channel of EphA4. An attempt was done to estimate the dissociation constants for the binding of the two compounds by fitting the HSQC peak tracings at different compound concentrations (Liu et al. (2006) Biochemistry 45, 7171-84). However, accurate data fitting was impossible because at high compound concentrations the HSQC peaks for the residues with large shifts disappeared.

Further attempts to identify intermolecular NOE connectivities between EphA4 and the compounds were not successful because the presence of the compounds appeared to induce significant NMR line-broadening, which even caused the disappearance of the EphA4 intra- and inter-residue NOEs. On the other hand, with progressive addition of the EphA4 protein, all ¹H resonance peaks of the two molecules underwent line broadening and gradual shifting in one-dimensional NMR spectra. This indicates that the free and bound forms of the two molecules were in fast exchange on the chemical shift timescale and also indicates that the entire molecules were either directly or indirectly affected by binding to EphA4, consistent with their small size.

Molecular docking—The absence of intermolecular NOEs between the EphA4 ligand-binding domain and the two molecules made it impossible to calculate the structures of their complexes with NMR distance constraints. As an alternative, HADDOCK docking strategy was used to construct models of the EphA4 ligand-binding domain in complex with the two molecules. HADDOCK is a recent but well-established docking procedure that makes use of NMR chemical shift perturbation data in conjunction with the CNS program to drive the molecular docking of protein-protein and protein-small molecule complexes. Interestingly, as shown in FIG. 10, each crystal asymmetric unit contains two EphA4 molecules A and B, which show large structural differences in the J-K loop. Interestingly, in solution the EphA4 ligand-binding domain is a monomer even at very high concentrations, as demonstrated by FPLC gel filtration, dynamic light scattering and analytic ultracentrifugation. Analysis of the NMR Cα, Cβ and Hα chemical shifts for the EphA4 ligand-binding domain in solution shows that the four residues Phe126-Val129 in the J-K loop preferentially form a short β-strand, as observed in molecule B. Furthermore, the NMR structure of the unliganded EphA4 ephrin-binding domain, which we have recently determined, is highly similar to those in the crystal and contains the short β-sheet observed in molecule B. Therefore, it is likely that molecule B in the crystal more closely represents the conformation of EphA4 in solution.

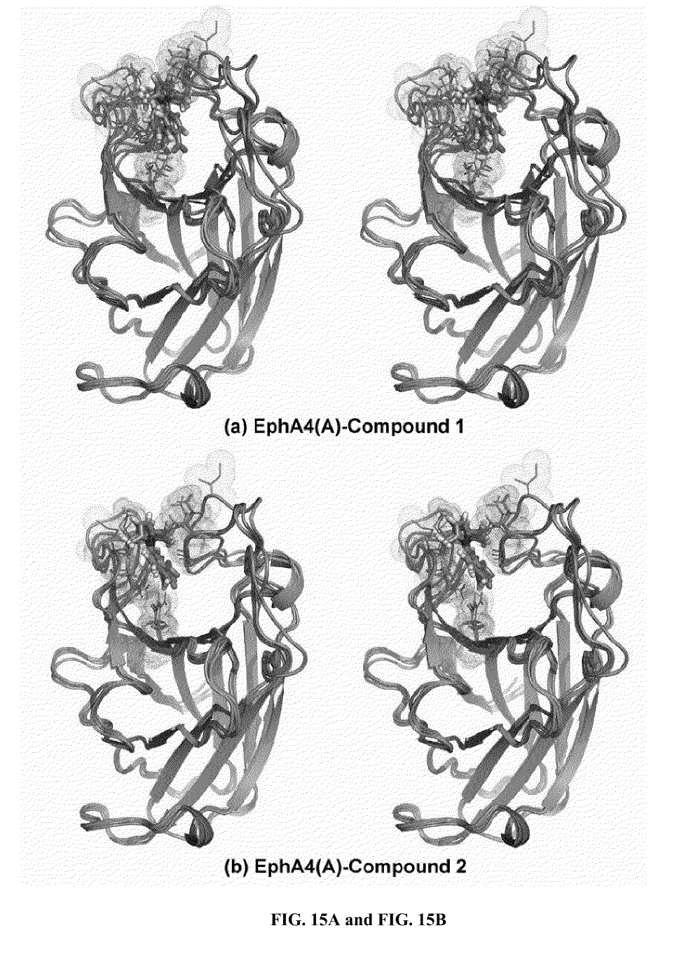

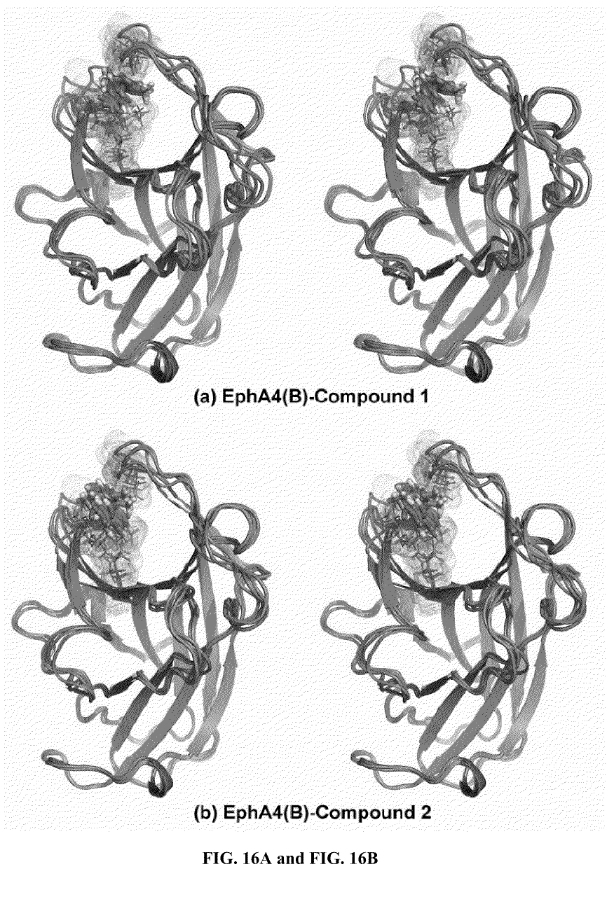

However, here to better capture the binding properties of the compounds with EphA4, EphA4 molecules A and B were used separately to construct the models of the complexes by using the HADDOCK docking procedure. As a consequence, four models were obtained: EphA4(A)-compound 1, EphA4(A)-compound 2, EphA4(B)-compound 1, and EphA4(B)-compound 2. From the structures generated from each docking running, three with the lowest energies were selected for further display and analysis (FIGS. 15 and 16). As revealed from these models of the complexes, the two initial EphA4 A and B structures only need some local conformational rearrangements to accommodate the two small molecules. The average rms deviations between the 3 selected structures and the initial structure are relatively small: only ˜2.0 (all protein atoms) and 1.1 Å (protein backbone atoms) for EphA4(A)-compound 1; ˜2.1 (all protein atoms) and 1.2 Å (protein backbone atoms) for EphA4(A)-compound 2; ˜1.9 (all protein atoms) and 1.0 Å (protein backbone atoms) for EphA4(B)-compound 1; and ˜1.8 (all protein atoms) and 1.0 Å (protein backbone atoms) for EphA4(B)-compound 2. If not considering the D-E and J-K loops, the rms deviation values reduce to ˜0.8 (all protein atoms) and 0.3 Å (protein backbone atoms) for EphA4(A)-compound 1; ˜0.8 (all protein atoms) and 0.3 Å (protein backbone atoms) for EphA4(A)-compound 2; ˜0.9 (all protein atoms) and 0.4 Å (protein backbone atoms) for EphA4(B)-compound 1; and ˜0.8 (all protein atoms) and 0.3 Å (protein backbone atoms) for EphA4(B)-compound 2.

Strikingly, as seen in FIGS. 15 and 16, despite starting from two different EphA4 structures, in all four models the two small molecules occupy a similar cavity of the high affinity ligand-binding channel of both EphA4 structures A and B. The two small molecules interact mainly with residues Ile31-Met32 in the D-E loop, Gln43 in the D-E β-strand and Ile131-Gly132 in the J-K loops, all of which have significant chemical shift differences (CSDs) in the NMR HSQC titration (FIGS. 14C and 14D). In contrast, despite being set as “active residues” in the docking calculations, residues Ile39 in the D-E loop and Asp123 in the J-K loop do not show direct contact with the two small molecules in any of the models. The HADDOCK docking procedure has been previously reported to correctly identify the residues most likely to form the binding pocket (Dominguez et al. (2003). J. Am. Chem. Soc. 125, 1731-1737; de Vries et al. Proteins. 69, 726-733 (2007); Zhang et al. (2006) J Mol. Biol. 363, 188-200). Thus, the chemical shift perturbations observed for Asp123 and Ile39 probably represent a secondary effect of binding-induced rearrangements of the D-E and J-K loops.

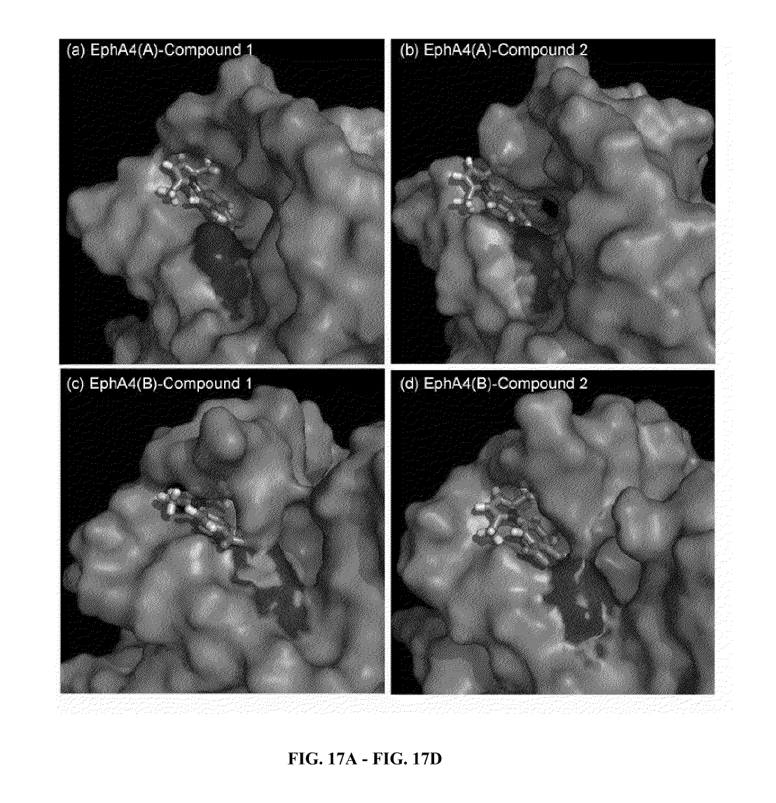

As shown in FIG. 17, a close examination of all the model structures reveals that the pyrrole and benzene rings of the two small molecules stack onto the hydrophobic surface formed by residues Ile31 and Met32 in the D-E loop. Moreover, the pyrrole ring is sandwiched by the hydrophobic side chains of Ile31-Met32 in the D-E loop and those of Ile 131 in the J-K loop. On the other hand, one of the methyl groups on the pyrrole ring inserts into the hydrophobic patch between the Ile31 and Met32 side chains and the other methyl group is in close contact with the Ile131 side chain. These interactions emphasize the importance of the two methyl groups on the pyrrole ring, which is completely consistent with the structure-activity relationship analysis of a series of small molecules with a pyrrolyl benzene scaffold (see Example 1).

In all 12 selected models, the carboxylic and hydroxyl groups on the benzene ring always orient towards the side chain of the EphA4 residue Gln43. Detailed analysis indicates that in all these models at least one hydrogen bond forms between the oxygen atoms of the carboxylic or hydroxyl groups and the side chain amide protons of Gln43. In some structures, even two hydrogen bonds can be identified between them. This observation could explain why removal of either the carboxylic or the hydroxyl group causes a dramatic loss in the activity of some of the modified compounds (see Example 1). Taken together, the docking results imply that the pyrrole and benzene rings, the two groups on the pyrrole ring, and the groups on the benzene ring are all important for the binding of small molecules with a 2,5-dimethylpyrrolyl benzene scaffold to the EphA4 ligand-binding domain. It has been discovered that groups on the pyrrole ring and the groups on the benzene ring can be varied within limits.

### 3. Discussion

The extensive involvement of the Eph receptor-ephrin interaction in various pathologies indicates that the main interface between the two proteins can serve as a new target for drugs. Previous studies reveal that the Eph receptor-ephrin interaction is mediated by two binding sites in the ligand-binding domain of the Eph receptor. One is a high affinity binding site, which includes a hydrophobic channel that is mainly constituted by the convex sheet of four β-strands and the D-E and J-K loops and that accommodates the protruding G-H loop of the ephrin. The other is a separate low affinity binding site (Himanen et al. (2007) Curr Opin Cell Biol 19(5), 534-542; Himanen et al. (2001) Nature 414, 933-938; Himanen et al. (2004) Nat. Neurosci. 7, 501-9; Chrencik et al. (2006) J Biol. Chem. 281, 28185-92). In particular, the high affinity hydrophobic channel of the receptor appears to be highly amendable for targeting by small molecule antagonists. However, previously-identified small molecules including a natural product from green tea (Caligiuri et al. (2006) Chem Biol 13, 711-722; Karaman et al. (2008) Nat Biotechnol 26, 127-132; Miyazaki et al. (2008) Bioorganic & medicinal chemistry letters 18, 1967-1971; Kolb et al. (2008) Proteins (10.1002/prot.22028);Tang et al. (2007) J Nutr Biochem 18, 391-399) all seem to target the intracellular kinase domain of the Eph receptors. Only now two small molecules with a 2,5-dimethylpyrrolyl benzene scaffold have been successfully identified in a high throughput screen (see Example 1). The fact that the two compounds competitively inhibit ephrin binding to EphA4 result strongly suggests that the two compounds occupy the ephrin-binding channel, thus directly competing with ephrins in binding with the EphA4 receptor. Therefore, it was of significant interest to define the structural mechanism by which the two compounds interact with the EphA4 receptor.

To achieve this, the EphA4 ligand-binding domain in the free state was crystallized and its structure was determined. This represents the first structure determined for the ligand-binding domain of an Eph receptor of the A subclass. In the crystal, each asymmetric unit contains two EphA4 molecules that show some large structural differences in the J-K loop due to their differential packing interactions with other EphA4 molecules in the neighboring asymmetric units. In solution, however, the EphA4 ligand-binding domain was found to be monomeric. The EphA4 ligand-binding domain adopts the same jellyroll β-sandwich architecture that was previously reported for the EphB2 and EphB4 ligand-binding domains. Interestingly, despite belonging to the Eph receptor A subclass, the core β-stranded regions of EphA4 bear a high similarity to those of the EphB2 and EphB4 receptors. Nevertheless, large variations do exist in the loop regions. For example, a short 310 helix is formed in the H-1 loop of EphA4. This helix has not been observed in the EphB receptors, which have a 4-residue insert in this loop. There are also dramatic differences in the D-E and J-K loops. Because large variations in the positioning of the D-E and J-K loops have also been observed in the different EphB structures previously determined in the free state or in complex with an ephrin or peptide ligands, this can reflect the intrinsic flexibility of the D-E and J-K loops, which can be needed to accommodate the binding of different ligands.

Isothermal titration calorimetry, circular dichroism, NMR and computational docking was used to characterize the possible binding interactions of the EphA4 ligand-binding domain with the two small molecules that inhibit the binding of peptide and ephrin ligands. The isothermal titration calorimetry results show that both small molecules bind to the EphA4 ligand-binding domain with similar Kd values in the micromolar range. On the other hand, consistent with the modest binding affinity of the compounds, the circular dichroism results indicate that binding of the two small molecules does not induce significant structural changes in the EphA4 ligand-binding domain. To identify the EphA4 residues involved in the binding of the two small molecules, a large set of NMR spectra was collected and succeeded in obtaining sequential assignments. This allowed us to identify the EphA4 residues that are significantly perturbed upon binding of the two small molecules by performing NMR HSQC titrations. Interestingly, only a few EphA4 residues showed significant perturbations upon binding, which include residues Ile31-Met32 in the D-E loop, Gln43 in the E β-strand, and Ile131-Gly132 in the J-K loop, in agreement with the small sizes of the two small molecules.

The well-established HADDOCK docking procedure was used to construct models of the EphA4 ligand-binding domain in complex with the two small molecules. The docking results indicate that both molecules occupy a cavity of the high-affinity ephrin binding channel of EphA4 in a similar manner, by interacting mainly with EphA4 residues in the E strand and the D-E and J-K loops. The results also reveal that all three building blocks of the 2,5-dimethylpyrrolylbenzene scaffold, namely the dimethylpyrrole ring, the benzene ring, and the carboxylic/hydroxyl groups on the benzene ring, are crucial for binding to the EphA4 ligand-binding domain. The pyrrole and benzene rings appear to play a key role in establishing stacked aromatic-hydrophobic interactions with Ile31-Met32 on the D-E loop and Ile131 on the J-K loop. The two methyl groups on the pyrrole ring further anchor the small molecules in between the D-E and J-K loops by using one methyl group to interact with the hydrophobic side chains of Ile31-Met32 and the other to interact with the hydrophobic side chain of Ile131. Furthermore, the carboxylic and hydroxyl groups on the benzene ring are involved in hydrogen bonding to the side-chain amide protons of Gln43 in EphA4, thus providing additional contacts with EphA4 as well as dictating the orientation of the small molecules in the complexes. Consequently, the docking models provide the structural rationale for the results of an extensive study on the structure-activity relationship of small molecules with a pyrrolyl benzene scaffold as EphA4 ligand-binding antagonists (see Example 1).

These results shed light on how such small molecules are capable of selectively targeting only EphA4 and the closely related EphA2 receptor (see Example 1). Sequence alignment reveals that some of the EphA4 residues that are perturbed by the binding are not conserved in other Eph receptors (Tables 3 and 4). In particular, residues Ile31-Met32 are only presented in EphA4 and EphA2 but not other Eph receptors, which can be at least partly responsible for the high binding-selectivity of the two molecules for the EphA4 and EphA2 receptors.

TABLE 3
Sequence alignment of EphA4 and other EphA receptors.

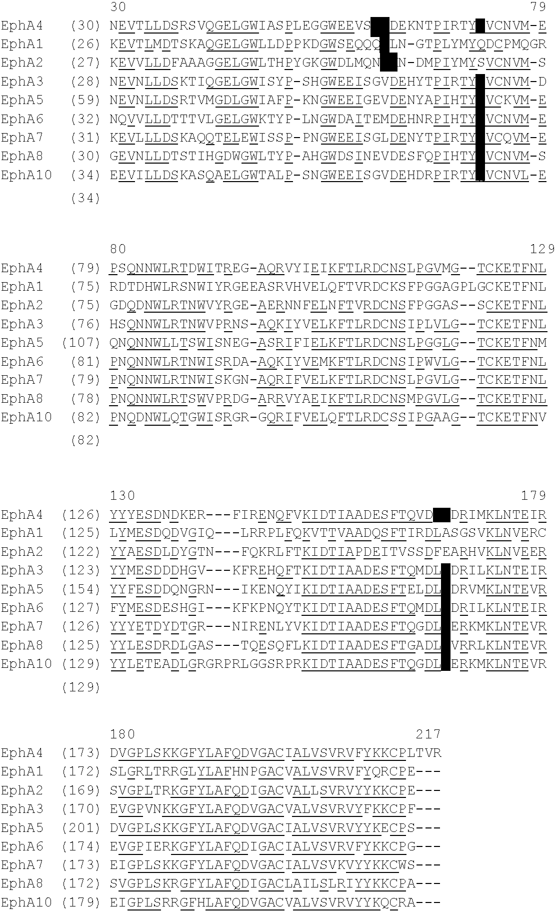

The sequences of EphA4, EphA1, EphA2, EphA3, EphA5, EphA6, EphA7, EphA8, EphA10, EphB1, EphB2, EphB3, EphB4, and EphB6 are SEQ ID NOs: 3 to 16, respectively.

TABLE 4
Sequence alignment of EphA4 and EphB receptors.

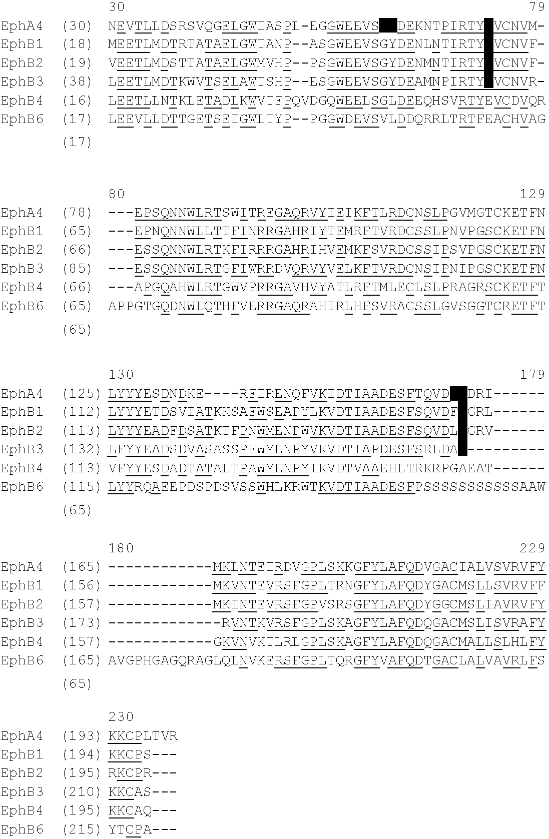

Tables 3 and 4 show the sequence alignment of the ligand binding domains of Eph receptors. Table 3. Sequence alignment of EphA4 and other EphA receptors. Table 4. Sequence alignment of EphA4 and EphB receptors. The highly conserved residues are underlined. The EphA4 residues important for binding the two small molecules antagonists identified in the present study, and the corresponding identical residues in other Eph receptors, are highlighted. Interestingly, EphA4 residues Ile31 (corresponding to residue 60 in the numbering of full length EphA4) and Met32 (corresponding to residue 60 in the numbering of full length EphA4), which are critical for binding the two small molecules, are only found in EphA2. The residues are numbered based on the full length receptors according to the sequences from GenBank. The alignments were obtained using the AlignX program in the Vector NTI software suite. The sequences of EphA4, EphA1, EphA2, EphA3, EphA5, EphA6, EphA7, EphA8, EphA10, EphB1, EphB2, EphB3, EphB4, and EphB6 are SEQ ID NOs:3 to 16, respectively.

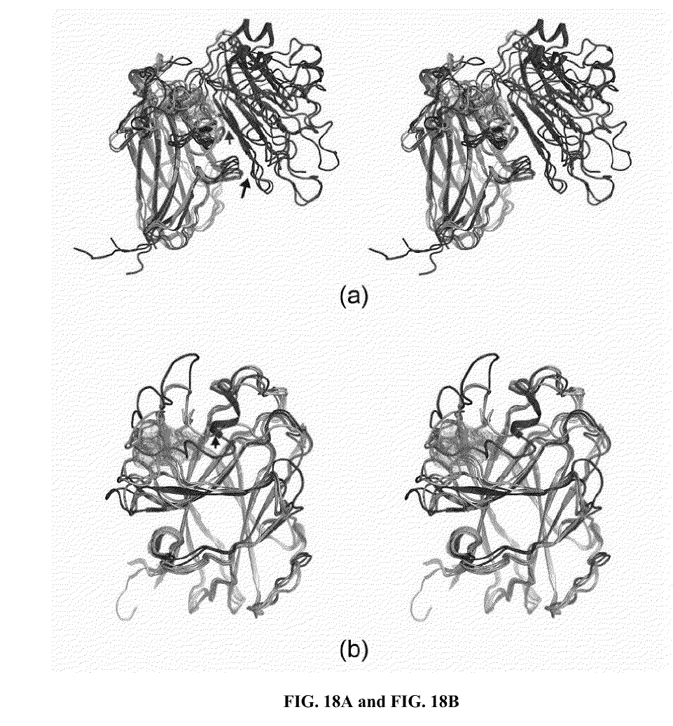

The results can also explain why the two small molecules bind to EphA4 with a medium affinity. First, EphA4 residues Ile31-Met32 and Ile131, which are critical for binding, are from the D-E and J-K loops. These loops are relatively flexible, as indicated by previous crystal structures and our NMR¹⁵N heteronuclear NOE data (to be published). Second, as shown in FIG. 18A, the two small molecules only occupy a portion of the EphA4 ligand-binding channel, which in EphB2 and EphB4 is occupied by the tip of the G-H loop of the ephrin ligands, corresponding to residues Pro122-Asn123-Leu124-Trp125-Gly126-Leu127 for ephrin-B2 and Pro127-Phe128-Ser129-Leu130-Gly131-Phe132 for ephrin-A5 (Himanen et al. (2007) Curr Opin Cell Biol 19(5), 534-542; Himanen et al. (2001) Nature 414, 933-938; Himanen et al. (2004) Nat. Neurosci. 7, 501-9; Chrencik et al. (2006) J Biol. Chem. 281, 28185-92). In contrast, interactions occurring outside of the high-affinity binding pocket of the Eph receptor are totally absent in the case of the small molecules. These interactions include those between the ephrin G β-strand and the Eph receptor D and E β-strands and A-C loop (Himanen et al. (2007) Curr Opin Cell Biol 19(5), 534-542; Himanen et al. (2001) Nature 414, 933-938; Himanen et al. (2004) Nat. Neurosci. 7, 501-9; Chrencik et al. (2006) J Biol. Chem. 281, 28185-92). Even within the high-affinity binding channel, a large portion of the key Eph receptor-ephrin interactions is absent in the EphA4-small molecule complexes due to the small size of the dimethylpirrole derivatives. For example, NMR titrations did not detect strong interactions between the two small molecules and the EphA4 G and M β-strands.

Furthermore, as shown in FIG. 18B, the interaction interface between EphA4 and the two compounds is also smaller than the interaction interfaces between the EphB2 and EphB4 receptors and their respective peptide ligands (Chrencik et al. (2006) Structure. 14, 321-30; Chrencik et al. (2007) J Biol. Chem. 282, 36505-13). For example, the two small molecules do not interact with the EphA4 disulfide bridge linking Cys45 and Cys53, whereas this interaction was found to be conserved in all the EphB structures in complex with either ephrins or antagonistic peptides (Chrencik et al. (2007) J Biol. Chem. 282, 36505-13).

### 4. Conclusion

The results confirm the binding interaction between the EphA4 ligand-binding domain and two novel small molecule antagonists with a 2,5-dimethylpyrrolyl benzene scaffold. Furthermore, NMR titrations were utilized to map out the residues involved in the interaction and used this information to construct models of the EphA4 ligand-binding domain in complex with the two small molecules. These models provide a structural rational for the results of an extensive structure-activity study on a large set of small molecules with a pyrrolyl benzene scaffold and for the high binding selectivity but relatively weak affinity of the compounds. Based on this model, modifications to the compounds and their derivatives that enhance interactions with the EphA4 G and M β-strands can be used to improve the binding activity and specificity of the EphA4 antagonists with a 2,5-dimethylpyrrolyl benzene scaffold.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds, reference to “the compound” is a reference to one or more compounds and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

1. A method of treating a subject, the method comprising
administering to the subject an EphA2/4 inhibitor.

2. The method of claim 1, wherein the EphA2/4 inhibitor is a compound of Formula I: 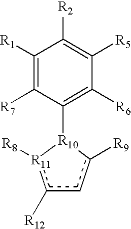
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₀ is N or C,
wherein R₁₁ is O or C,
wherein R₁₀ and R₁₁ are not both simultaneously C,
wherein if R₁₀ is N, then R₈ and R₉ are each independently —CH₃, —CH₂—CH₃, or —CH₂—CH₂—CH₃, and R₁₁ is C,
wherein if R₁₁ is O, then R₈ and R₉ are each H, and R₁₀ is C,
wherein R₁₂ is H, or 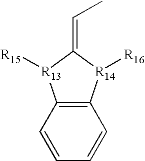
wherein R₁₃ and R₁₄ are each C or S,
wherein R₁₅ and R₁₆ are each ═O or absent,
wherein if R₁₃ is S, then R₁₅ is absent,
wherein if R₁₃ is C, then R₁₅ is ═O,
wherein if R₁₄ is S, then R₁₆ is absent, wherein if R₁₄ is C, then R₁₆ is ═O.

3. The method of claim 1, wherein the EphA2/4 inhibitor is a compound of Formula II: 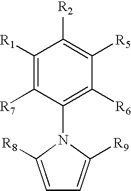
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₈ and R₉ are each independently —CH₃, —CH₂—CH₃, or —CH₂—CH₂—CH₃.

4. The method of claim 1, wherein the EphA2/4 inhibitor is a compound of Formula III: 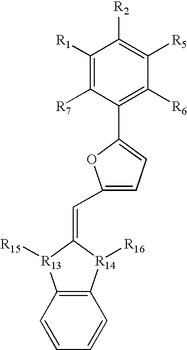
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₃ and R₁₄ are each C or S,
wherein R₁₅ and R₁₆ are each ═O or absent,
wherein if R₁₃ is S, then R₁₅ is absent,
wherein if R₁₃ is C, then R₁₅ is ═O,
wherein if R₁₄ is S, then R₁₆ is absent, and
wherein if R₁₄ is C, then R₁₆ is ═O.

5. The method of claim 2, wherein if R₁₃ is S, then R₁₄ is C, and if R₁₄ is S, then R₁₃ is C.

6. The method of claim 2, wherein R₁₃ and R₁₄ are each C and R₁₅ and R₁₆ are each ═O.

7. The method of claim 2, wherein R₅ is —OH, R₆ is —H, and R₇ is —H.

8. The method of claim 2, wherein R₅ is —H, R₆ is —H, and R₇ is —OH.

9. The method of claim 2, wherein R₅ is —H, R₆ is —H, and R₇ is —H.

10. The method of claim 2wherein R₈ is —CH₃ and R₉ is —CH₂—CH₃.

11. The method of claim 2, wherein R₈ is —CH₂—CH₃ and R₉ is —CH₃.

12. The method of claim 2, wherein R₈ is —CH₃ and R₉ is —CH₃.

13. The method of claim 2, wherein R₃ is —OH, and wherein R₄ is —COOH, —CH₂—COOH, or —CH₂—CH₂—COOH.

14. The method of claim 13, wherein R₄ is —COOH.

15. The method of claim 1, wherein the subject has suffered or is at risk of suffering nerve injury.

16. The method of claim 1, wherein the subject is suffering or is at risk of suffering cancer.

17. The method of claim 16, wherein the subject has cancer cells in which EphA2 is activated above a threshold level.

18. The method of claim 16 further comprising measuring EphA2 receptor activity in cancer cells prior to administering the EphA2/4 inhibitor.

19. The method of claim 16, wherein the subject is suffering or is at risk of suffering tumor angiogenesis.

20. A method of identifying compounds, the method comprising
determining the binding characteristics of a test compound in the presence and absence of an EphA2/4 inhibitor, wherein if the test compound exhibits noncompetitive binding with the EphA2/4 inhibitor and if the test compound inhibits EphA4 receptor activity in the absence of the EphA2/4 inhibitor, then the test compound is identified as a noncompetitive binder of EphA4.

21. The method of claim 20, wherein the EphA2/4 inhibitor is a compound of Formula I: 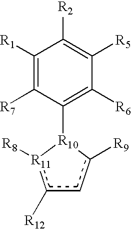
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₀ is N or C,
wherein R₁₁ is O or C,
wherein R₁₀ and R₁₁ are not both simultaneously C,
wherein if R₁₀ is N, then R₈ and R₉ are each independently —CH₃, —CH₂—CH₃, or —CH₂—CH₂—CH₃, and R₁₁ is C,
wherein if R₁₁ is O, then R₈ and R₉ are each H, and R₁₀ is C,
wherein R₁₂ is H, or

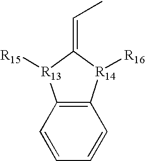

22. The method of claim 20, wherein the EphA2/4 inhibitor is a compound of Formula II: 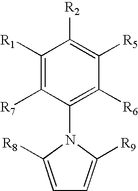
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₈ and R₉ are each independently —CH₃, —CH₂—CH₃, or —CH₂—CH₂—CH₃.

23. The method of claim 20, wherein the EphA2/4 inhibitor is a compound of Formula III: 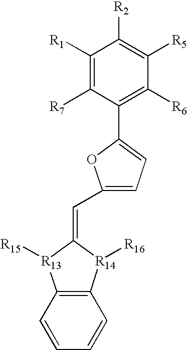
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₃ and R₁₄ are each C or S,
wherein R₁₅ and R₁₆ are each ═O or absent,
wherein if R₁₃ is S, then R₁₅ is absent,
wherein if R₁₃ is C, then R₁₅ is ═O,
wherein if R₁₄ is S, then R₁₆ is absent, and
wherein if R₁₄ is C, then R₁₆ is ═O.

24. The method of claim 20 further comprising linking the noncompetitive binder to an EphA2/4 inhibitor via a linker to form a linked EphA2/4 binder.

25. The method of claim 24 further comprising administering to a subject the linked EphA2/4 binder.

26. The method of claim 24, wherein the subject has suffered or is at risk of suffering nerve injury.

27. The method of claim 24, wherein the subject is suffering or is at risk of suffering cancer.

28. The method of claim 27, wherein the subject has cancer cells in which EphA2 is activated above a threshold level.

29. The method of claim 27 further comprising measuring EphA2 receptor activity in cancer cells prior to administering the EphA2/4 inhibitor.

30. The method of claim 27, wherein the subject is suffering or is at risk of suffering tumor angiogenesis.

31. A method of identifying compounds that interact with EphA4, the method comprising
bringing into contact a test compound, an EphA2/4 inhibitor composition, and an EphA4 receptor, wherein the EphA2/4 inhibitor composition comprises an EphA2/4 inhibitor; and
detecting unbound EphA2/4 inhibitor composition, wherein a given amount of unbound EphA2/4 inhibitor composition indicates a compound that interacts with EphA4.

32. The method of claim 31, wherein the EphA2/4 inhibitor composition further comprises a moiety linked to the EphA2/4 inhibitor.

33. The method of claim 32, wherein the moiety further comprises a detectable agent.

34. The method of claim 31, wherein the EphA2/4 inhibitor is a compound of Formula I: 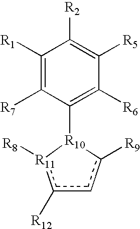
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₀ is N or C,
wherein R₁₁ is O or C,
wherein R₁₀ and R₁₁ are not both simultaneously C,
wherein if R₁₀ is N, then R₈ and R₉ are each independently —CH₃, —CH₂—CH₃, or —CH₂—CH₂—CH₃, and R₁₁ is C,
wherein if R₁₁ is O, then R₈ and R₉ are each H, and R₁₀ is C,
wherein R₁₂ is H, or

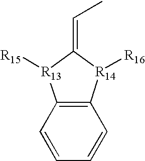

35. The method of claim 31, wherein the EphA2/4 inhibitor is a compound of Formula II: 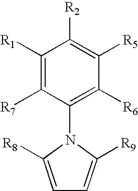
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₈ and R₉ are each independently —CH₃, —CH₂—CH₃, or —CH₂—CH₂—CH₃.

36. The method of claim 31, wherein the EphA2/4 inhibitor is a compound of Formula III: 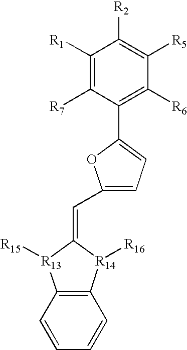
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₃ and R₁₄ are each C or S,
wherein R₁₅ and R₁₆ are each ═O or absent,
wherein if R₁₃ is S, then R₁₅ is absent,
wherein if R₁₃ is C, then R₁₅ is ═O,
wherein if R₁₄ is S, then R₁₆ is absent, and
wherein if R₁₄ is C, then R₁₆ is ═O.

37. The method of claim 31 further comprising administering to a subject the test compound that interacts with EphA4.

38. The method of claim 37, wherein the subject has suffered or is a risk of suffering nerve injury.

39. The method of claim 37, wherein the subject is suffering or is a risk of suffering cancer.

40. The method of claim 39, wherein the subject has cancer cells in which EphA2 is activated above a threshold level.

41. The method of claim 39 further comprising measuring EphA2 receptor activity in cancer cells prior to administering the EphA2/4 inhibitor.

42. The method of claim 39, wherein the subject is suffering or is at risk of suffering tumor angiogenesis.

43. A method of identifying a subject as having EphA4 receptor activity of interest, the method comprising
measuring EphA4 receptor activity in the cell of a subject in the presence of an EphA2/4 inhibitor, wherein the subject has EphA4 receptor activity of interest if the measured EphA4 receptor activity differs from a reference EphA4 receptor activity by more than a threshold amount.

44. The method of claim 43, wherein the reference EphA4 receptor activity is a normal EphA4 receptor activity of a normal cell.

45. The method of claim 43, wherein the reference EphA4 receptor activity is a non-pathological EphA4 receptor activity.

46. The method of claim 43, wherein the measured EphA4 receptor activity is lower than the reference EphA4 receptor activity by more than the threshold amount.

47. A method of identifying a subject as having EphA2 receptor activity of interest, the method comprising
measuring EphA2 receptor activity in the cell of a subject in the presence of an EphA2/4 inhibitor, wherein the subject has EphA2 receptor activity of interest if the measured EphA2 receptor activity differs from a reference EphA2 receptor activity by more than a threshold amount.

48. The method of claim 47, wherein the reference EphA2 receptor activity is a normal EphA2 receptor activity of a normal cell.

49. The method of claim 47, wherein the reference EphA2 receptor activity is a non-pathological EphA2 receptor activity.

50. The method of claim 47, wherein the measured EphA2 receptor activity is lower than the reference EphA2 receptor activity by more than the threshold amount.

51. The method of claim 43, wherein the EphA2/4 inhibitor is a compound of Formula I: 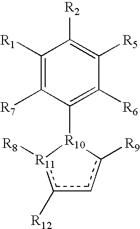
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₀ is N or C,
wherein R₁₁ is O or C,
wherein R₁₀ and R₁₁ are not both simultaneously C,
wherein if R₁₀ is N, then R₈ and R₉ are each independently —CH₃, —CH₂—CH₃, or —CH₂—CH₂—CH₃, and R₁₁ is C,
wherein if R₁₁ is O, then R₈ and R₉ are each H, and R₁₀ is C,
wherein R₁₂ is H, or

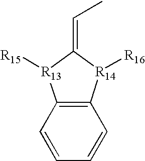

52. The method of claim 43, wherein the EphA2/4 inhibitor is a compound of Formula II: 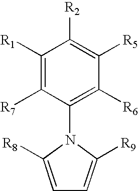
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₈ and R₉ are each independently —CH₃, —CH₂—CH₃, or —CH₂—CH₂—CH₃.

53. The method of claim 43, wherein the EphA2/4 inhibitor is a compound of Formula III: 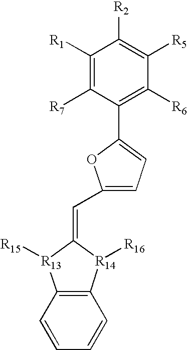
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₃ and R₁₄ are each C or S,
wherein R₁₅ and R₁₆ are each ═O or absent,
wherein if R₁₃ is S, then R₁₅ is absent,
wherein if R₁₃ is C, then R₁₅ is ═O,
wherein if R₁₄ is S, then R₁₆ is absent, and
wherein if R₁₄ is C, then R₁₆ is ═O.

54. The method of claim 43 further comprising administering to a subject the EphA2/4 inhibitor.

55. The method of claim 54, wherein the subject has suffered or is a risk of suffering nerve injury.

56. The method of claim 54, wherein the subject is suffering or is a risk of suffering cancer.

57. The method of claim 56, wherein the subject has cancer cells in which EphA2 is activated above a threshold level.

58. The method of claim 56 further comprising measuring EphA2 receptor activity in cancer cells prior to administering the EphA2/4 inhibitor.

59. The method of claim 56, wherein the subject is suffering or is at risk of suffering tumor angiogenesis.

60. A pharmaceutical composition comprising an EphA2/4 inhibitor and a pharmaceutically acceptable carrier.

61. The composition of claim 60, wherein the EphA2/4 inhibitor is a compound of Formula I: 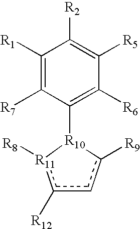
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₀ is N or C,
wherein R₁₁ is O or C,
wherein R₁₀ and R₁₁ are not both simultaneously C,
wherein if R₁₀ is N, then R₈ and R₉ are each independently —CH₃, —CH₂—CH₃, or —CH₂—CH₂—CH₃, and R₁₁ is C,
wherein if R₁₁ is O, then R₈ and R₉ are each H, and R₁₀ is C,
wherein R₁₂ is H, or

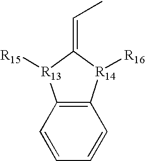

62. The composition of claim 60, wherein the EphA2/4 inhibitor is a compound of Formula II: 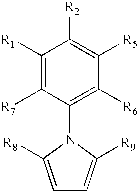
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₈ and R₉ are each independently —CH₃, —CH₂—CH₃, or —CH₂—CH₂—CH₃.

63. The composition of claim 60, wherein the EphA2/4 inhibitor is a compound of Formula III: 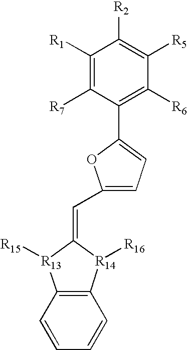
wherein R₁ is R₃ or R₄,
wherein R₂ is R₃ or R₄,
wherein R₁ and R₂ are not both R₃ or both R₄,
wherein R₃ is —H, —OH, or —SH,
wherein R₄ is —COOH, —CH₂—COOH, or —CH₂—CH₂—COOH,
wherein R₅, R₆, and R₇ are each independently —H or —OH,
wherein R₁₃ and R₁₄ are each C or S,
wherein R₁₅ and R₁₆ are each ═O or absent,
wherein if R₁₃ is S, then R₁₅ is absent,
wherein if R₁₃ is C, then R₁₅ is ═O,
wherein if R₁₄ is S, then R₁₆ is absent, and wherein if R₁₄ is C, then R₁₆ is ═O.


SEQUENCE LISTING (16 sequences)

SEQ ID NO: 1 - DNA, 30 bp, Homo sapiens
  misc_binding (1)..(30)
ggatccaatgaagttaccttattggattcc

SEQ ID NO: 2 - DNA, 28 bp, Homo sapiens
  misc_binding (1)..(28)
ctcgagtcagcggactgtgagtggacac

SEQ ID NO: 3 - protein, 181 aa, Homo sapiens
  PEPTIDE (1)..(181)
NEVTLLDSRSVQGELGWIASPLEGGWEEVSIMDEKNTPIRTYQVCNVMEPSQNNWLRTDWITREGAQRVYIEIKFTLRDCNSLPGVMGTCKETFNLYYYESDNDKERFIRENQFVKIDTIAADESFTQVDIGDRIMKLNTEIRDVGPLSKKGFYLAFQDVGACIALVSVRVFYKKCPLTVR

SEQ ID NO: 4 - protein, 181 aa, Homo sapiens
  PEPTIDE (1)..(181)
KEVTLMDTSKAQGELGWLLDPPKDGWSEQQQILNGTPLYMYQDCPMQGRRDTDHWLRSNWIYRGEEASRVHVELQFTVRDCKSFPGGAGPLGCKETFNLLYMESDQDVGIQLRRPLFQKVTTVAADQSFTIRDLASGSVKLNVERCSLGRLTRRGLYLAFHNPGACVALVSVRVFYQRCPE

SEQ ID NO: 5 - protein, 177 aa, Homo sapiens
  PEPTIDE (1)..(177)
KEVVLLDFAAAGGELGWLTHPYGKGWDLMQNIMNDMPIYMYSVCNVMSGDQDNWLRTNWVYRGEAERNNFELNFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPE

SEQ ID NO: 6 - protein, 177 aa, Homo sapiens
  PEPTIDE (1)..(177)
NEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPF

SEQ ID NO: 7 - protein, 177 aa, Homo sapiens
  PEPTIDE (1)..(177)
NEVNLLDSRTVMGDLGWIAFPKNGWEEIGEVDENYAPIHTYQVCKVMEQNQNNWLLTSWISNEGASRIFIELKFTLRDCNSLPGGLGTCKETFNMYYFESDDQNGRNIKENQYIKIDTIAADESFTELDLGDRVMKLNTEVRDVGPLSKKGFYLAFQDVGACIALVSVRVYYKECPS

SEQ ID NO: 8 - protein, 177 aa, Homo sapiens
  PEPTIDE (1)..(177)
NQVVLLDTTTVLGELGWKTYPLNGWDAITEMDEHNRPIHTYQVCNVMEPNQNNWLRTNWISRDAAQKIYVEMKFTLRDCNSIPWVLGTCKETFNLFYMESDESHGIKFKPNQYTKIDTIAADESFTQMDLGDRILKLNTEIREVGPIERKGFYLAFQDIGACIALVSVRVFYKKCPF

SEQ ID NO: 9 - protein, 177 aa, Homo sapiens
  PEPTIDE (1)..(177)
KEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWS

SEQ ID NO: 10 - protein, 177 aa, Homo sapiens
  PEPTIDE (1)..(177)
GEVNLLDTSTIHGDWGWLTYPAHGWDSINEVDESFQPIHTYQVCNVMSPNQNNWLRTSWVPRDGARRVYAEIKFTLRDCNSMPGVLGTCKETFNLYYLESDRDLGASTQESQFLKIDTIAADESFTGADLGVRRLKLNTEVRSVGPLSKRGFYLAFQDIGACLAILSLRIYYKKCPA

SEQ ID NO: 11 - protein, 180 aa, Homo sapiens
  PEPTIDE (1)..(180)
EEVILLDSKASQAELGWTALPSNGWEEISGVDEHDRPIRTYQVCNVLEPNQDNWLQTGWISRGRGQRIFVELQFTLRDCSSIPGAAGTCKETFNVYYLETEADLGRGRPRLGGSRPRKIDTIAADESFTQGDLGERKMKLNTEVREIGPLSRRGFHLAFQDVGACVALVSVRVYYKQCRA

SEQ ID NO: 12 - protein, 181 aa, Homo sapiens
  PEPTIDE (1)..(181)
MEETLMDTRTATAELGWTANPASGWEEVSGYDENLNTIRTYQVCNVFEPNQNNWLLTTFINRRGAHRIYTEMRFTVRDCSSLPNVPGSCKETFNLYYYETDSVIATKKSAFWSEAPYLKVDTIAADESFSQVDFGGRLMKVNTEVRSFGPLTRNGFYLAFQDYGACMSLLSVRVFFKKCPS

SEQ ID NO: 13 - protein, 181 aa, Homo sapiens
  PEPTIDE (1)..(181)
VEETLMDSTTATAELGWMVHPPSGWEEVSGYDENMNTIRTYQVCNVFESSQNNWLRTKFIRRRGAHRIHVEMKFSVRDCSSIPSVPGSCKETFNLYYYEADFDSATKTFPNWMENPWVKVDTIAADESFSQVDLGGRVMKINTEVRSFGPVSRSGFYLAFQDYGGCMSLIAVRVFYRKCPR

SEQ ID NO: 14 - protein, 177 aa, Homo sapiens
  PEPTIDE (1)..(177)
LEETLMDTKWVTSELAWTSHPESGWEEVSGYDEAMNPIRTYQVCNVRESSQNNWLRTGFIWRRDVQRVYVELKFTVRDCNSIPNIPGSCKETFNLFYYEADSDVASASSPFWMENPYVKVDTIAPDESFSRLDAGRVNTKVRSFGPLSKAGFYLAFQDQGACMSLISVRAFYKKCAS

SEQ ID NO: 15 - protein, 184 aa, Homo sapiens
  PEPTIDE (1)..(184)
LEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQ

SEQ ID NO: 16 - protein, 203 aa, Homo sapiens
  PEPTIDE (1)..(203)
LEEVLLDTTGETSEIGWLTYPPGGWDEVSVLDDQRRLTRTFEACHVAGAPPGTGQDNWLQTHFVERRGAQRAHIRLHFSVRACSSLGVSGGTCRETFTLYYRQAEEPDSPDSVSSWHLKRWTKVDTIAADESFPSSSSSSSSSSSAAWAVGPHGAGQRAGLQLNVKERSFGPLTQRGFYVAFQDTGACLALVAVRLFSYTCPA